(19) United States
(12) Patent Application Publication
(10) Pub. No.: US 2005/0019787 A1
(43) Pub. Date: Jan. 27, 2005

(54) Apparatus and methods for analyzing and characterizing nucleic acid sequences

(75) Inventors: Anthony J. Berno, San Jose, CA (US); Karel Konvicka, Palo Alto, CA (US); David A. Hinds, Mountain View, CA (US); Nila Patil, Woodside, CA (US); Naiping Shen, Saratoga, CA (US); David Stern, Mountain View, CA (US); John Sheehan, Mountain View, CA (US)

Correspondence Address: PERLEGEN SCIENCES, INC.;LEGAL DEPARTMENT, 2021 STIERLIN COURT, MOUNTAIN VIEW, CA, 94043, US

(73) Assignee: Perlegen Sciences, Inc., a Delaware Corporation, Mountain View, CA (US)

(21) Appl. No.: 10/768,788

(22) Filed: Jan. 30, 2004

Related U.S. Application Data
(60) Provisional application No. 60/460,329, filed on Apr. 3, 2003.
(60) Provisional application No. 60/518,107, filed on Nov. 6, 2003.

(51) Int. Cl. C12Q 1/68; G06F 19/00; G01N 33/48; G01N 33/50

(52) U.S. Cl. 435/6; 702/20

(57) ABSTRACT

Computer-implemented methods, data processing apparatus and computer program codes for characterizing an interrogation position, such as a SNP position, in a nucleic acid sequence are provided herein. Computer-implemented methods involving various statistical analyses are provided for characterizing an interrogation position as being associated with a phenotypic trait of interest.

This application claims the benefit of U.S. Provisional Application No. 60/460,329 filed Apr. 3, 2003, incorporated by reference herein.

### BACKGROUND OF THE INVENTION

The variations in many of the characteristics of people have a genetic basis (i.e., are the result of differences in their DNA), which changes the expression of proteins relevant to the characteristics. Sometimes the differences involve one or more base pairs. The single base pair differences are called single nucleotide polymorphisms, or SNPs for short.

Some SNPs do not appear to be associated with physical changes in people, but others may. Significant effort has been made to identify as many SNPs as possible in the human genome. SNPs that do not themselves change protein expression and cause disease may be close on the chromosome to harmful mutations. Because of this proximity, such SNPs may be correlated with harmful but unknown mutations and serve as markers for them. Such markers can help in discovery and identification of the mutations and aid the development of therapeutic drugs. Also, analyzing shifts in SNP allele frequency among different groups of people may help population geneticists to trace the evolution of the human race and to unravel the connections between different ethnic groups and races.

While a significant number of SNPs have been identified, it is not a simple matter to determine how variations in phenotypic characteristics, e.g. diseases, are related to individual SNPs, combinations of SNPs or sequences of SNPs, within a gene or an entire genotype. One method of trying to determine the genetic basis of a particular characteristic is an association study. Association studies attempt to identify the differences at a genetic level between people in which a characteristic of their phenotype (e.g. a particular disease) is present (“case” or “affected”) and people in which the characteristic is apparently not present (“control” or “unaffected”) in order to identify the genetic basis for the characteristic.

A significant portion of human DNA (over 99%) is invariant and SNPs make up a small amount of genetic information. Moreover, a large majority of SNPs do not appear to be associated with a particular characteristic of the phenotype in a simple way. Rather, expression of a characteristic in a phenotype appears to be determined in subtle and complex ways by many different and various combinations of several SNP alleles. Therefore, association studies are trying to identify a small and subtle signature within a very large amount of genetic information.

Nucleic acid arrays have been successfully used in association studies. One significant advance in association studies is outlined in, for example, U.S. patent application Ser. No. 10/106,097, filed Mar. 26, 2002 entitled “Methods for Genomic Analysis”, which is incorporated herein by reference. According to these methods, a significant number of SNPs are first identified across the genome, and optionally grouped into SNP haplotype groups. Thereafter, the SNPs are screened in case and control groups to identify the SNPs that correlate with a phenotype.

The following patents and patent applications are incorporated herein by reference in their entirety: U.S. Pat. No. 5,800,992, filed Jun. 25, 1996, entitled “Method of Detecting Nucleic Acids”; U.S. Pat. No. 5,861,242, filed Jan. 9, 1997, entitled “Array of Nucleic Acid Probes on Biological Chips for Diagnosis of HIV and Method of Using the Same”; U.S. Pat. No. 6,228,593, filed Jan. 14, 2000, entitled “Computer-Aided Probability Base Calling for Arrays of Nucleic Acid Probes on Chips”; U.S. patent application Ser. No. 09/922,492, filed Aug. 3, 2001, entitled “High Performance Wafer Scanning”; U.S. patent application Ser. No. 10/131,832, filed Apr. 24, 2002, entitled “Methods for Reducing Complexity of Nucleic Acid Samples”; U.S. patent application Ser. No. 10/236,480, filed Sep. 5, 2002, entitled “Methods for Amplification of Nucleic Acids”; and U.S. patent application Ser. No. 10/341,832, filed Jan. 14, 2003, entitled “Short Range PCR Primer Picking”.

While meeting with significant success, it is desirable to further improve the speed and efficiency with which association studies are performed, and further decrease the cost of such studies. The present invention meets these and other needs.

### BRIEF SUMMARY OF THE INVENTION

The present invention provides computer-implemented methods, apparatus, and systems for obtaining and analyzing nucleic acid sequence data. In particular, methods, apparatus, computer systems and programs are provided for analyzing data from nucleic acid arrays to characterize bi-allelic markers, such as single nucleotide polymorphisms (SNPs), in nucleic acid sequences.

In one aspect of the invention, a computer-implemented method is provided for characterizing a position in a nucleic acid segment or sequence. In one embodiment, the method comprises: inputting into a computer system a first measure of relative allele frequency at the interrogation position in the nucleic acid segment derived from a first sample collected from a first group of n individuals, wherein n is an integer equal to or larger than 2; inputting into the computer system a second measure of relative allele frequency at the interrogation position in the nucleic acid segment derived from a second sample collected from a second group of m individuals, wherein m is an integer equal to or larger than 2; and analyzing in the computer system the first measure and the second measure to characterize the interrogation position, for example, as being associated with a phenotypic characteristic of interest.

In another aspect of the invention, a data processing apparatus is provided for characterizing an interrogation position in a nucleic acid segment. The data processing apparatus comprises: a data processor; a storage device holding computer readable code in communication with the data processor, the computer readable code including: computer code which determines a first measure of relative allele frequency at the interrogation position in the nucleic acid segment derived from a first sample collected from a first group of n individuals, wherein n is an integer equal to or larger than 2; computer code which determines a second measure of relative allele frequency at the interrogation position in the nucleic acid segment derived from a second sample collected from a second group of m individuals, wherein m is an integer equal to or larger than 2; and computer code which analyzes the first measure and the second measure to characterize the interrogation position, for example, as being associated with a phenotypic characteristic of interest.

In yet another aspect of the invention, a computer readable medium is provided for holding computer readable code for characterizing a position in a nucleic acid segment and for carrying out the processes of: determining a first measure of relative allele frequency at the interrogation position in the nucleic acid segment derived from a first sample collected from a first group of n individuals, wherein n is an integer equal to or larger than 2; determining a second measure of relative allele frequency at the interrogation position in the nucleic acid segment derived from a second sample collected from a second group of m individuals, wherein m is an integer equal to or larger than 2; and analyzing the first measure and the second measure to characterize the interrogation position as being associated with a phenotypic characteristic of interest.

The above-described embodiments and other aspects of the invention are described in detail below in the section of “Detailed Description of the Invention”.

### BRIEF DESCRIPTION OF THE DRAWINGS

The invention, together with further advantages thereof, may best be understood by reference to the following description taken in conjunction with the accompanying drawings in which:

FIG. 1 shows a schematic diagram comparing DNA segments of three individuals with the corresponding segment from the human genome sequence to illustrate types of genetic variations;

FIG. 2 shows a schematic diagram of DNA segments for members of a control group and a case group in an association study to illustrate a disease model wherein the “x” indicates the presence of the disease allele, which can be the reference allele or the alternate allele;

FIG. 3 shows a flow chart illustrating an experimental protocol according to an aspect of the invention;

FIG. 4A shows a computer system suitable for carrying out data processing methods and processes according to various aspects of the invention;

FIG. 4B shows a schematic block diagram of data processing apparatus parts of the computer system shown in FIG. 4A;

FIG. 5 shows a flow chart illustrating data analysis processes according to the present invention;

FIGS. 6A, 6B, 6C and 6D respectively show schematic diagrams illustrating probe tiling patterns on an array for a forward reference sequence, a reverse reference sequence, a forward alternate sequence and a reverse alternate sequence;

FIGS. 7A and 7B respectively show schematic diagrams illustrating parts of the probe sequences for the forward reference and forward alternate tilings shown in FIGS. 6A and 6C;

FIG. 8 shows a flow chart illustrating a data processing method for carrying out a data conformance evaluation as used in the general method illustrated in FIG. 5;

FIG. 9 shows a flow chart illustrating a method for carrying out a relative allele frequency determination for a tiling as used in the general method illustrated in FIG. 5;

FIG. 10 shows a flow chart illustrating a process for analyzing relative allele frequency data to characterize SNP positions;

FIG. 11 shows a flow chart illustrating a further process for analyzing relative allele frequency data to characterize SNP positions;

FIGS. 12A, 12B and 12C respectively show distributions of relative allele frequency data for a case group, a control group and the combined data illustrating the data analysis process shown in FIG. 11;

FIG. 13 shows a plot of differences in relative allele frequency data for SNPs against SNP position, illustrating a characterization validation step of the data analysis process;

FIG. 14 shows a flow chat illustrating an optional SNP characterization validation process part of the data analysis process;

FIG. 15 shows distributions of differences in relative allele frequency for repeated experiments illustrating alternative data analysis processes for characterizing SNPs;

FIG. 16 shows a flow chart illustrating an embodiment of a data analysis process for characterizing SNPs as illustrated by FIG. 15;

FIG. 17 shows two graphical representations illustrating consistent an inconsistent variations in independent measures of relative allele frequency from the forward and reverse tilings illustrating a further analysis process;

FIG. 18 shows a flow chart illustrating a further data analysis process as illustrated by FIG. 17;

FIG. 19 shows a flow chart illustrating a further data analysis process using a rank test;

FIG. 20 shows a diagram illustrating a ranking step of the ranking data analysis process illustrated by FIG. 19;

FIG. 21 shows a flow chart illustrating a further data analysis process using experimentally paired data items:

FIGS. 22A and 22B respectively show distributions of data items and the distribution of differences in paired data items; and

FIG. 23 shows a flow chart illustrating a data analysis process using a paired t-test as illustrated by FIGS. 22A and 22B.

FIG. 24 shows an intensity plot of the intensity of light from the reference probes and the intensity of light from the alternate probes.

FIG. 25 shows a plot with clustering of relative allele frequency.

FIG. 26 shows a flow chart illustrating a process for calculating an AS/AB ratio.

In the Figures, like reference numerals refer to like components and elements.

### DETAILED DESCRIPTION OF THE INVENTION

### I. General

The present inventions relate to computer programs for analysis and characterization of nucleic acid sequences, for example, bi-allelic markers in nucleic acid sequences. The inventions are particularly applicable in the field of association studies but are not limited to such applications. Rather, they can be used whenever it is desirable to reliably characterize a nucleotide position in a nucleic acid sequence. Such a nucleotide position is hereinafter referred to as “an interrogation position” which can be any nucleotide position within a nucleic acid sequence of interest, including but not limited to a SNP position or a nucleotide position within an expressed sequence tag (EST), a genome survey sequence (GSS), or a sequence tagged site (STS) that is operationally unique in a genome.

1. SNPs in the Human Genome Sequence

The invention is particularly applicable in analyzing and characterizing human DNA sequences collected-from different groups of individuals.

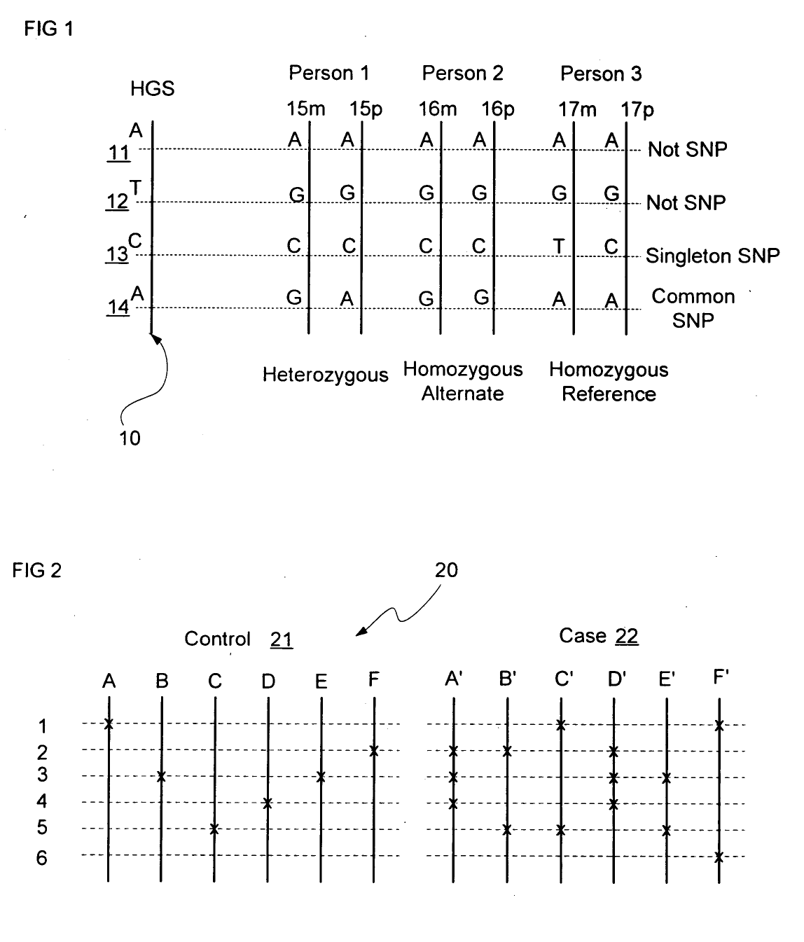

FIG. 1 shows a schematic diagram of a segment of DNA from the human genome sequence (HGS) together with the corresponding segment of DNA from three different people. Line 10 represents a segment of DNA from the human genome sequence having four nucleotide positions 11, 12, 13, and 14 represented by the nucleotides A, T, C, and A, respectively. A single nucleotide polymorphism (hereinafter SNP) generally relates to a genetic variation between individuals that occurs at a single nucleotide (or nitrogenous base) position in the DNA of an organism and is polymorphic (has multiple forms) within a population of the individuals. For example, a human SNP can be a variation in the base at a particular nucleotide position in an allele of an individual as compared to the base at the corresponding nucleotide position in the HGS. A nucleotide position can be identified by its absolute position in a sequence or by the sequence of bases in the locale of the position, as it is common for the ‘same position’ in a DNA sequence to be in different actual positions in the DNA sequences of different HGS versions. Irrespective of how the position is identified or defined, at a SNP position in a DNA sequence an individual will, in general, have a particular allele, i.e. the base C, G, T or A, present at the SNP position (absent any nucleotide deletion mutations or chemically-modified nucleotides).

The published human genome sequences are composites made up of DNA sequences from a variety of people. One ‘correct’ DNA sequence cannot be assumed, but rather such composites can be considered a reference DNA sequence with which the DNA sequences of individuals can be compared. The human genome includes mutations which may or may not be common to the population in general. In general, “common” SNPs will be most useful herein, e.g., those that occur in more than 10% of the population, although in some embodiments common and rare SNPs may be utilized. As illustrated in FIG. 1, the four nucleotide positions of the human genome segment 10 have four loci, 11, 12, 13, and 14, and the alleles present at these loci are A, T. C and A, respectively. It will be appreciated that FIG. 1 is exemplary only.

FIG. 1 also shows DNA segments for the maternal (m) and paternal (p) chromosomes for three different people (labeled as 15m, 15p; 16m, 6p; and 17m, 17p for Person 1, 2 and 3, respectively). The four alleles shown for each chromosome correspond to the same loci as shown for human genome segment 10. An association study attempts to identify SNPs that may be associated with a phenotypic characteristic.

At the first position 11 in the reference HGS segment 10, Persons 1, 2 and 3 have the same base A as the base in the reference sequence A on both chromosome segments. Therefore, in general, this position is not considered to be a SNP position (based on the data in FIG. 1) since the nucleotides are all identical to the reference nucleotide. In practice, it may be that there are some people in the world that do not have base A at this position, but based on three people comprising the population under consideration for the purposes of this discussion, position 11 can be considered not to be a SNP position.

At the second position 12, the reference sequence base T is different to the base G in all three people. This position may not be considered a common SNP, as all the population have the same different base, which may indicate that the reference sequence has a unique mutation at this position which is not present in any other person (recalling that the human genome sequence is merely an ad hoc reference sequence and not the only ‘correct’ sequence). Or, there may simply be a sequencing error in the reference sequence.

At the third position 13, Person 3 has a different base T in its maternal chromosome (17m) as compared to the reference base C in its paternal chromosome (17p) at the same position. This location is designated as a singleton SNP since it occurs infrequently in the population (e.g., only once in one chromosome within a population of 3 individuals in this example).

The fourth position can be considered a common SNP position. The reference allele in the HGS reference segment is A and Person 3 has the reference allele on both chromosomes 17m and 17p. Such a person has a homozygous reference genotype. Person 1 has the reference allele A on the paternal chromosome (15p) and the alternate allele G on the maternal chromosome (15m). This person has a heterozygous genotype. Person 2 has the alternate allele G on both maternal and paternal chromosomes (16m and 16p) and has a homozygous alternate genotype. Position 14 can be considered to be a common SNP as the alternate allele is common in the population. Although common SNPs are often interrogated in association studies to characterize as being associated with the phenotypic characteristic under investigation, rare and singleton SNPs can also be interrogated for the same purpose.

SNPs are most frequently biallelic, i.e.,. are characterized by having only two possible alleles. In this example the reference allele is A and the alternate allele is G, and not T or C. For each supposed SNP position identified in the human genome the reference and alternate alleles have been experimentally determined.

Preferably, the invention is applied to characterize SNP positions in nucleic acid segments collected from different groups of individuals in genotype-phenotype association studies. Association studies are useful for the identification of genetic components (e.g., genes and their regulatory regions, and unexpressed regions of genomic DNA) associated with phenotypic traits. These genetic components may be directly or indirectly involved in the manifestation of the phenotypic trait, whether causative or predictive. These genetic components may occur at one specific locus in the genome or at multiple loci on the same or different chromosomes.

2. Genotype-Phenotype Association Studies

Association studies may be accomplished by determining the genotypes (e.g. which allele is present at each of a given set of polymorphisms, which are typically SNPs) of individuals with the phenotype of interest (for example, individuals exhibiting a particular disease or individuals who respond in a particular manner to administration of a drug) and comparing the genotypes of these individuals to the genotypes of a control group of individuals who do not exhibit the phenotype of interest.

To facilitate an association study, a computer implemented method is provided herein for characterizing a single or multiple interrogation positions, such as SNP positions, in nucleic acid segments collected from case and control groups of individuals. According to the method, relative allele frequencies for the case and control groups at an interrogation position in the nucleic acid segments are input into a computer and analyzed in order to assess if the interrogation position can be characterized as being associated with the phenotypic characteristic of interest. Optionally, the genotypes of two or more populations of individuals may be compared on the basis of, for example, age, gender, ethnicity, or geographic location.

The phenotype of interest may be a disease, condition or other characteristic found in humans. A disease may be manifested as the presence of signs and/or symptoms in an individual or patient that are generally recognized as abnormal. Diseases may be diagnosed and categorized based on pathological changes. Signs may include any objective evidence of a disease such as changes that are evident by physical examination of a patient or the results of diagnostic tests which may include, among others, laboratory tests to determine the presence of variances or variant forms of certain genes in a patient. Symptoms are signs or indications in a patient of a disease, disorder, or condition that differs from normal function, sensation, or appearance, which may include, without limitation, physical disabilities, morbidity, pain, and other changes from the normal condition exhibited by an individual. The phenotypic characteristic of interest may also be an individual's reaction to administration of an agent (e.g., a drug, food, or alcohol) or other interventions, adverse or beneficial. The person's reaction may be susceptible or resistant to the effect(s) of the agent.

Examples of diseases or conditions of interest include, but are not limited to, neural disorders, connective tissue disorders, immune system disorders, skeletal/bone disorders, hematologicaL disorders, muscular disorders, hormonal disorders, infection by pathogens such as viruses, bacteria and parasites, reproductive disorders, gastrointestinal disorders, pulmonary disorders, cardiovascular disorders, renal disorders, proliferative disorders, and cancerous disease conditions. Specific examples of diseases include, but are not limited to, Alzheimer's disease, Huntington's disease, Creutzfeldt-Jacob syndrome, vascular dementia, fragile-X syndrome, primary or acquired immunodeficiency, viral infection, bacterial infection, parasitic infection, pneumonia, meningitis, herpes zoster, diabetes mellitus, ulcer, gastric reflux, liver diseases, autoimmune diseases such as multiple sclerosis, rheumatoid arthritis, Graves' disease, systemic lupus erythematosus, diabetes mellitus, aseptic meningitis, systemic scleroderma, autism, allergies, asthma, adult-onset idiopathic hypoparathyroidism and membranous glomerulonephritis, kidney failure, metabolic and congenital kidney disorders, heart disease, obesity, limb ischemia, leukemia, Hodgkin's disease, non-Hodgkin's lymphoma, and neuplasma located in the colon, abdomen, bone, breast, digestive system, liver, pancreas, peritoneum, endocrine glands (adrenal, parathyroid, pituitary, testicle, ovary, thymus, thyroid), eye, head and neck, nervous (central and peripheral), pelvis, skin, soft tissue, spleen, thorax, and urogenital tract.

Although SNPs within the coding regions of DNA may have functional consequences, SNPs identified in other portions of a gene or in nongenetic regions can provide identification of genetic polymorphisms of functional significance in an association study. For example, SNPs in the promoter, enhancers, silencers, or other regulatory sites, introns, splice sites, and 3′ untranslated regions of a gene may affect the transcription, translation and message processing of the genetic information thereby affecting protein levels or concentrations, availability, or activity. Many of these alterations may or may not affect protein cellular functions. However, the polymorphism may affect the response of an individual to a particular drug or treatment, for example, by interfering with the therapeutic effect on a target protein or nucleic acid. Thus, SNPs in both coding regions of DNA and noncoding regions of the genome can be analyzed in an association study, e.g., to identify SNPs associated with a particular response to a drug treatment. In one embodiment, loci across the part or all of the genome are evaluated without regard to whether any particular region is believed before the study to have a functional role. Another embodiment, SNPs in a particular region or regions, such as in the vicinity of a gene, which may be a coding or noncoding region(s), can be evaluated without previous knowledge that the region is associated with the phenotype of interest.

Once a genetic locus or multiple loci in the genome are associated with a particular phenotypic trait, such as disease susceptibility or drug response, the gene or genes or regulatory elements responsible for the trait may be identified. These genes or regulatory elements may then be used as therapeutic targets for the treatment of the disease, for more efficacious medicine specifically tailored to the genetic makeup of an individual, to develop diagnostics for identifying individuals with the trait, for further studying the biological pathways underlying the trait, or in a variety of other applications.

FIG. 2 is a schematic diagram 20 showing six DNA segments for a control group 21 of six people (A-F) and case group 22 of six different people (A′-F′) involved in an association study for a phenotypic trait, such as a particular disease, illness or condition exhibited by the case group. An exemplary disease model will be described with reference to FIG. 2. In FIG. 2 only a single chromosome is illustrated so as to simplify the explanation, but it will be apparent to a person of ordinary skill in the art in light of this description how the model would be applied to take into account the pairs of chromosomes present in humans.

The control group 21 includes six people A-F chosen at random and each believed not to have the disease that is the subject of the study. The case group 22 includes six people A′-F′ each believed to have the disease. For the purpose of illustration, assume there are in fact six SNP positions, 1-6, which are associated with the disease, out of all the SNP positions in the human genome sequence. At each of the six disease SNP positions, the presence of a particular allele (designated by “x”) is involved with the occurrence of the disease. However, for this example, the individuals who display the trait of interest have at least two of the “x” alleles.

The purpose of an association study is to characterize each SNP position analyzed as being associated with the phenotypic characteristic of interest. In other words, in the context of the model illustrated in FIG. 2, the goal of an association study is to identify the six SNP positions involved in the disease, without any prior knowledge of if the associated loci exist, where they are in the genome, or how many loci are associated. When a SNP position has been identified as associated with the disease, it can then be determined whether it is the reference allele or alternate allele which is the allele involved in the disease.

This can be achieved by measuring and analyzing the relative allele frequency, P, of a SNP position for the case and control groups. That is, the allele frequencies of the alleles that are involved in the phenotypic trait of interest will be significantly higher in the case group (which displays said trait) than in the control group (which does not). Exemplary embodiments of methods for carrying out experiments and analyzing experimental data will now be described in greater detail.

### II. Methods for Carrying Out Association Studies

1. General Scheme

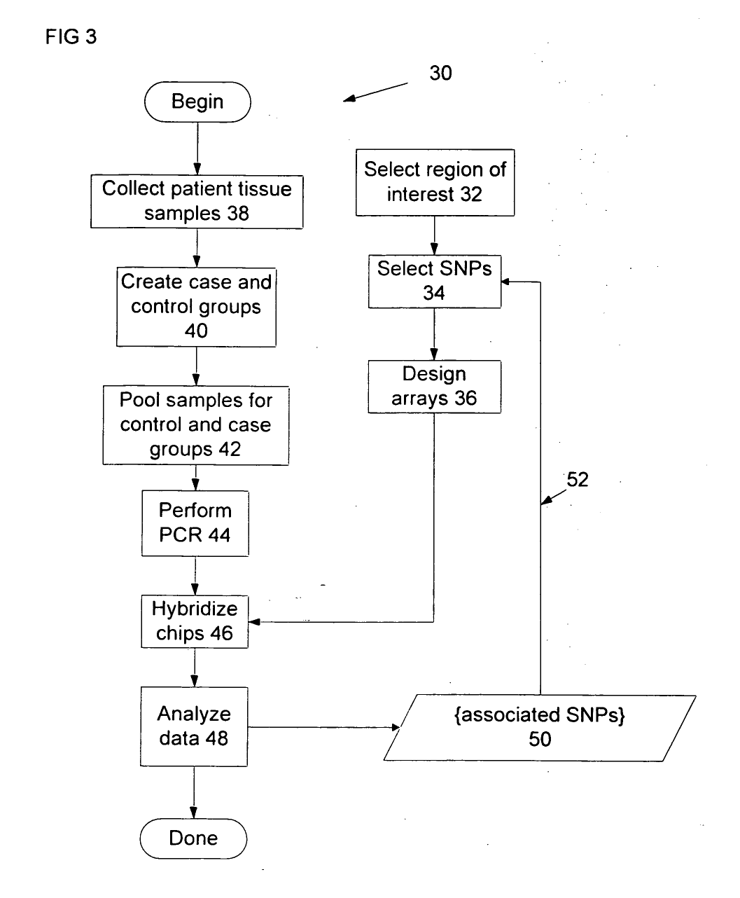

FIG. 3 is a flowchart 30 illustrating the steps of a method for carrying out an association study to characterize those SNPs associated with a particular phenotypic characteristic, such as a disease. A number of the activities illustrated in the flow chart can be carried out in parallel and the flow chart is merely one way of illustrating the activities involved. Some of the activities may be carried out in a different order and/or combined as part of other activities as will be apparent. The flow chart shows two parallel sequences of activities which may or may not be carried out in parallel.

Either the whole human genome or a region of interest within the human genome is selected at step 32 and the SNP positions in that region are identified and selected at step 34 using any available source of human genome data that contains information regarding SNPs, such as, for example, the U.C. Santa Cruz Human Genome Browser Gateway (http://genome.ucsc.edu/cgi-bin/hgGateway) or the NCBI dbSNP website (http://www.ncbi.nlm.nih.gov/SNP/). The reference and alternate allele data for the selected SNPs is used at step 36 to design and manufacture the assays, such as the probe arrays to be used in the experiment as will be described in greater detail below. The actual SNPs selected to be used in the experiment will depend on the nature, e.g. comprehensiveness, of the experiment being carried out, and may include common, rare, and singleton SNPs. Preferably, common SNPs are used. Preferably more than 300,000 SNPs are used, preferably more than 600,000 SNPs are used, and preferably more than 1 million SNPs are used across the genome.

Biological samples, for example, blood or tissue samples, are taken from individuals who are either “affected” (exhibit the phenotype of interest) or “unaffected” (do not exhibit the phenotype of interest) at step 38. The individuals who are affected form the “case” group, and the individuals who are unaffected form the “control” group at step 40. The number of individuals in the case group and in the control group may be the same or different, each preferably equal to or larger than 5, optionally equal to or larger than 20, optionally equal to larger than 50, optionally equal to or larger than 100, optionally equal to or larger than 200, optionally equal to or larger than 500, optionally equal to or larger than 1000, optionally between 10-100,000, optionally between 100-10,000, or optionally between 200-1,000. Further, studies may involve multiple phenotypes and, as such, a given population may be subdivided differently depending on the phenotype of interest under study. For example, one population may be studied for both heart disease and stroke, and while some individuals may be in the case group for both, other individuals may be in the case group for heart disease and in the control group for stroke, or vice versa. Thus, the two case groups from the same population would have different constituent individuals depending on which phenotype was being used as the criteria for dividing the population.

The samples for the association study may be pooled either as tissue samples from the case or control group, or optionally by pooling genetic materials from the group (i.e., nucleic acid such as genomic DNA, mitochondrial DNA, extragenomic DNA, cDNA, or RNA), and optionally by further amplification of the pooled genetic materials. The pooled nucleic acid may be amplified separately for the case and control groups, optionally under the same conditions of amplification reactions (e.g., PCR), optionally in PCR reactions run in parallel, and optionally in PCR reactions carried out by the same person. The amplicons of genetic materials from the case and control groups may be both labeled with a detectable marker (e.g., cychrome, fluorescein, Alexa-488, radioisotopes and biotin). Alternatively, the amplicons of genetic materials from the case may be labeled with a detectable marker (e.g., cychrome) different from the detectable marker on amplicons of the control group (e.g., fluorescein). Two-color labeling is described in U.S. Pat. No. 6,342,355, incorporated herein by reference in its entirety. The amplicons from the case and control groups can be pooled together to form one sample if different labels are used, in one embodiment. In certain embodiments, the genetic materials are labeled with a detectable marker prior to application to an array; in certain embodiments, the genetic materials are further stained with a detectable marker after application to the array.

The tissue samples for the members of the control group are pooled at step 42 which provides a pool of genetic material of the individuals in the population of the control group. Similarly, the tissue samples of the case group are pooled at step 42 to provide a pool of genetic material of the individuals in the population of the case group. Optionally, genetic materials may be isolated from each individual in the case or control group and pooled together to provide a sample for the association study.

By pooling tissues or nucleic acids from individuals in each group and characterizing SNPs in the pool, the cost of an association study can be significantly reduced by not scanning all SNPs separately in each individual. In one embodiment, two or more “rounds” are performed in an association study. In one round, all or a large fraction of the SNPs selected at step 34 are evaluated in a pooled manner. A subset of those SNPs (for example, those that are found to be in regions of the genome that are potentially involved in manifestation of the phenotype of interest or those that, based on the pooled analysis, appear to be associated with the phenotype of interest) are used in a second round in which each individual is genotyped for only those potentially associated SNPs. The efficiency of the association study is significantly increased by characterizing the SNPs that have been pooled together as compared to individually characterizing each SNP separately.

For example, for a study with case and control groups of 100 individuals each, 100-fold fewer experiments are required if samples are pooled for each group. In the case where two rounds are performed, the number of SNPs that need to be further characterized is significantly reduced e.g., from millions in the first round to a few thousands or fewer in the second round.

Target nucleic acids in the samples may be prepared using any technique known to those skilled in the art, which, preferably, results in the production of a nucleic acid molecule sufficiently pure to determine the presence or absence of one or more variations (e.g. SNPs) at one or more locations in the nucleic acid molecule. It is noted that although genomic DNA is often as a target nucleic acid, other nucleic acid samples may be used, such as RNA (e.g., mRNA) and cDNA, and both human and non-human (e.g., plant, bacterial, fungal, avian, reptilian, amphibian, fish or non-human mammalian) nucleic acid samples.

The DNA sequences of the case and control pools may be amplified by performing PCR reactions at step 44. Methods for performing PCR reactions are described in Innis et al. “PCR Protocols: a Guide to Methods and Applications”, Academic Press, Inc., 1990, and in U.S. patent application Ser. No. 10/042,492, filed Jan. 9, 2002, entitled “Methods for Amplification of Nucleic Acids”, both of which are incorporated herein by reference. The DNA samples from the case and control pool can be amplified as many times as required to provide enough DNA to allow statistically significant differences in SNP frequencies to be detected in the case and control groups. The control and case pools can each be amplified by two separate PCR reactions to provide sufficient pooled DNA case and control group samples to allow an experiment to be repeated, for example, three times for each PCR reaction, thereby providing six sets of experimental results for each group: three for each of the case and control groups from the first PCR reaction and three for each of the case and control groups from the second PCR reaction. Optionally, PCR may be performed on nucleic acid contained in a sample from each individual in the case or control group prior to the pooling of his/her sample with others in the group.

The SNPs are then assayed to determine the relative allele frequency of each SNP in the case and control groups. In one embodiment, the allelic frequency of SNPs or other genetic information can be detected by using oligonucleotide arrays. Depending on the number of SNPs to be screened, for example, oligonucleotide arrays with low, medium or high density can be employed. The density of the oligonucleotide array may be higher than 100 probes per square centimeters, optionally higher than 1000, optionally higher than 10,000, optionally higher than 1,000,000, optionally between 100-100,000,000, and optionally between 1,000,000-80,000,000 probes per square centimeters. Of course, other assays may also be utilized, such as sequencing methods, mass spectroscopy and electrophoresis.

Preferably, high-density oligonucleotide array chips or larger DNA probe array wafers (from which individual chips would otherwise be obtained by breaking up the wafer) are used in one embodiment of the invention. DNA probe array wafers generally comprise glass wafers on which high density arrays of DNA probes (short segments of DNA) have been formed. Each of these wafers can hold, for example, approximately 60 million or more DNA probes that are used to recognize DNA sequences. The recognition of sample DNA by the set of DNA probes on the glass wafer takes place through the mechanism of DNA hybridization. When a DNA sample hybridizes with an array of DNA probes, the sample binds to those probes that are complementary to the sample DNA sequence. By evaluating which probes hybridize to the sample DNA more strongly, it is possible to determine whether or not a known sequence of DNA is present in the sample DNA, for example, to detect the presence of a SNP.

The use of DNA probe arrays to obtain genetic information involves the following general steps: design and manufacture of DNA probe array wafers, preparation of the sample, hybridization of target DNA to the array, detection of hybridization events and data analysis to determine sequence. Preferred wafers are manufactured using a process adapted from semiconductor manufacturing to achieve cost effectiveness and high quality, and are available from Affymetrix, Inc. of California.

Probe arrays can be manufactured by a light-directed chemical synthesis process, which combines solid-phase chemical synthesis with photolithographic fabrication techniques as employed in the semiconductor industry. Using a series of photolithographic masks to define chip exposure sites, followed by specific chemical synthesis steps, the process constructs high-density arrays of oligonucleotides, with each probe in a predefined position in the array. Multiple probe arrays are synthesized simultaneously on a large glass wafer. This parallel process enhances reproducibility and helps achieve economies of scale.

Once fabricated, DNA probe arrays can be used to obtain genetic information about nucleic acid samples. The nucleic acid samples are tagged with a fluorescent reporter group by standard biochemical methods. The labeled samples are incubated with a DNA probe array, and segments of the samples bind, or hybridize, with complementary sequences on the DNA probe array. The DNA probe array is then scanned and the patterns of hybridization are detected by emission of light from the fluorescent reporter groups. Because the identity and position of each probe on the DNA probe array is known, the nature of the nucleic acid sequences in the sample applied to the DNA probe array can be determined. When these arrays are used for genotyping experiments, they may be referred to as genotyping arrays.

Once fabricated the arrays are ready for hybridization. The nucleic acid sample to be analyzed is isolated, amplified and labeled with a fluorescent reporter group. The labeled nucleic acid sample is then incubated with the array using a fluidics station and hybridization oven. After the hybridization reaction is complete, the array is inserted into the scanner, where patterns of hybridization are detected. The hybridization data are collected as light emitted from the fluorescent reporter groups already incorporated into the labeled nucleic acid, which is now bound to the probe array. Probes that most clearly match the labeled nucleic acid bind more of the nucleic acid, and hence accumulate more of the fluorescent signal than those that have mismatches. Since the sequence and position of each probe on the array are known, by complementarity, the identity of the nucleic acid sample applied to the probe array can be identified.

In reference to FIG. 3, in a first experiment, the amplicons of a one of the amplified case pools are hybridized at step 46 with one set of the designed genotyping arrays and the amplicons of a one of the amplified control pools are hybridized with another identical set of the designed genotyping arrays. The concentrations of each individual sample are carefully controlled such that they are previously known and, in preferred embodiments, equal, or approximately equal. The intensity of light emitted from the arrays is measured and recorded and is stored as a data file for subsequent data analysis at step 48. Methods and apparatus for measuring light emitted from the arrays are described in U.S. Pat. No. 6,586,750, filed Aug. 3, 2001, issued Jul. 1, 2003, entitled “High Performance Wafer Scanning”, which is incorporated herein by reference in its entirety for all purposes. The experiment can then be repeated or replicated using amplicons for the case and control pools from the same or another PCR reaction. In the example described above, the experiment is replicated six times for the same case and control groups, giving six data sets, each comprising case group data and control group data, for analysis at step 48. For those SNP alleles that are present in higher abundance in the “case” group than the “control” group, the corresponding probes in the assay will tend to emit a higher intensity of light than in the “control” group. For those SNPs where the frequency of a particular allele in the population is approximately equal in the case and control groups, the intensity of light emitted from the probes for such SNP alleles will be approximately the same for the case and control DNA probe arrays.

In another embodiment, the case and control pools are differentially labeled and combined into a single pool and are hybridized with a single set of the designed genotyping arrays. In this way case and control group data can be obtained from the same physical arrays. More than one set of genotyping arrays can be used to replicate the experiment. Labels that may be used include, but are not limited to, cychrome, fluorescein, Alexa-488, or biotin (in certain embodiments, later stained with phycoerythrin-streptavidin after hybridization). In certain embodiments, the genetic materials are labeled with a detectable marker prior to application to an array; in certain embodiments, the genetic materials are further stained with a detectable marker after application to the array. Two-color labeling is described in U.S. Pat. No. 6,342,355, incorporated herein by reference in its entirety. Each array may be scanned such that the signal from both labels is detected simultaneously, or may be scanned twice to detect each signal separately. It has been found that measured intensities for cychrome and fluorescein labeled pools can have a non-linear relationship. One origin of this effect is believed to be due to the saturation of sample molecules with cychrome. Without wishing to be bound by theory, it is believed that adjacent cychrome on the surface of the labeled molecules may interfere and reduce the amount of light emitted. Therefore, in one embodiment, only a single staining step is used for cychrome, rather than two stains. Alternatively, a reduced amount of cychrome can be used in the staining in order to reduce the non-linearity in detected intensity. In certain embodiments, fluorescein-labeled pools may be further stained with Alexa-488. By the comparing the intensity of the label corresponding to the case group to the intensity of the label corresponding to the control group at a specific probe on the array, one can determine if a particular SNP allele appears more commonly in the case or control group.

The data analysis at step 48 characterizes the SNPs as either being associated with the phenotype being studied or not associated with the phenotype being studied. In one embodiment, the method can reduce the initial set of SNPs to a much smaller set of associated SNPs, probably several orders of magnitude fewer, e.g. tens of thousands from hundreds of thousands. The result of the first set of experiments is a reduced set of SNPs comprising a set of SNP positions 50, which have been-characterized as likely being associated with the phenotype being studied. Of course, in this embodiment, one will use a rougher filter that will potentially capture many non-associated SNPs as well as associated SNPs.

Typically, the association study experiments are repeated, but using the reduced set of associated SNPs 50, in the selection 34 of the SNPs to be used in designing a second set of arrays. Hence a second iteration of the association study experiments may be performed characterizing fewer SNPs (e.g., thousands of SNPs) resulting from the first experiment than those in the first round (e.g., millions of SNPs). Hence, the actual hybridization experiments can be replicated an increased number of times to help reduce random variations in the experimental data due to, for example, sampling or experimental error.

In one preferred embodiment, the association study is performed by using individual samples with the genotyping array (or other assays), rather than using pooled samples. Analysis of the data at step 48 during this second iteration further reduces the set of previously characterized SNPs so that a more definitive characterization of SNPs associated with the disease can be obtained. For example, after the second analysis on the reduced set of SNPs a set of tens, hundreds or thousands of SNPs characterized as associated with the disease can be identified. The set of SNPs likely associated with the disease having been identified, the particular alleles at those SNP positions can be determined from the genotypes of the members of the case and control groups, providing an indication of the genetic basis of the disease or condition. These associated SNPs, their alleles and allelic frequencies, and the genetic regions in which these SNPs are found can be used for a wide variety of purposes, e.g., to screen for individuals who may be susceptible to the disease, to help in the development of medicaments and diagnostics, or in other applications.

For example, if the associated SNPs are located in the coding region of a gene, function of this gene may be implicated in the manifestation of the phenotype of interest (e.g. succeptibility or resistance to a disease). In one embodiment, the gene containing the associated SNPs can be cloned into an expression vector for expressing the encoded protein recombinantly. Optionally, the encoded protein may be expressed in its native cellular environment, or isolated from other cellular components. Various agents can be screened for the ability to modulate activities of the gene and its product, including but not limited to small molecule compounds, antisense molecules, ribozymes, triple helix molecules, antibodies and other protein or peptide modulators. If the gene encodes a receptor which has a known cognate ligand, agonists and antagonists against the receptor may be developed based on the structures of the receptor and/or ligand. The agonists and antagonists may be tested in an in vitro or in vivo assay, for example a cell-based assay expressing the gene product, alone or in competition with the cognate ligand. Changes in the activities of the gene or its product in the presence or absence of the agent can be monitored using various detection methods known in the art, including but not limited to hybridization arrays, radioisotope-labeling, immunochemical staining, and fluorescence-activated cell sorting (FACS). Depending on the role of the gene played in the onset and/or progression of the disease/condition, the agent that effectively promotes or inhibits the activities of the gene or its product can be selected and used as a lead agent to be further developed into a pharmaceutical agent.

If the associated SNPs are located in a regulatory region of a gene, control of the expression of the gene may be implicated in the manifestation of the phenotype of interest. Various agents can be screened for the ability to modulate expression of the gene, including but not limited to small molecule compounds, antisense molecules, ribozymes, triple helix molecules, antibodies and other protein or peptide modulators. For example, antibodies or a protein modulator of the gene expression may be developed by using a yeast-two-hybrid system to screen for molecules that interfere with the binding of a transcription factor for the gene, thereby altering the control mechanism of the regulatory region containing the associated SNPs. Changes in the expression levels of the associated gene in the cells can be monitored using various detection methods known in the art, including but not limited to hybridization arrays, radioisotope-labeling, immunochemical staining, and fluorescence-activated cell sorting (FACS). Depending on the role of the gene played in the onset and/or progression of the disease/condition, the agent that effectively increases or decreases expression levels of the gene can be selected and used as a lead agent to be further developed into a pharmaceutical agent.

Various methods can be used in the analysis at step 48 of the experimental data to characterize the SNPs, and several exemplary embodiments will be described below.

2. Methods for Analysis of Experimental Data

The different data analysis methods can be implemented wholly, or in part, by suitable computer processes. Certain embodiments of the present invention employ processes acting under control of instructions and/or data stored in or transferred through one or more computer systems. Embodiments of the present invention also relate to an apparatus for performing these operations. This apparatus may be specially designed and/or constructed for the required purposes, or it may be a general-purpose computer selectively activated or reconfigured by a computer program and/or data structure stored in the computer. The processes presented herein are not inherently related to any particular computer or other apparatus. In particular, various general-purpose machines may be used with programs written in accordance with the teachings herein, or it may be more convenient to construct a more specialized apparatus to perform the required method steps. A particular structure for a variety of these machines will appear from the description given below.

In addition, embodiments of the present invention relate to computer readable media or computer program products that include program instructions and/or data (including data structures) for performing various computer-implemented operations. Examples of computer-readable media include, but are not limited to, magnetic media such as hard disks, floppy disks, magnetic tape; optical media such as CD-ROM devices and holographic devices; magneto-optical media; semiconductor memory devices, and hardware devices that are specially configured to store and perform program instructions, such as read-only memory devices (ROM) and random access memory (RAM), and sometimes application-specific integrated circuits (ASICs), programmable logic devices (PLDs) and signal transmission media for delivering computer-readable instructions, such as local area networks, wide area networks, and the Internet. The data and program instructions of this invention may also be embodied on a carrier wave or other transport medium (e.g., optical lines, electrical lines, and/or airwaves).

For example, the program instruction of this invention may be installed in or is part of a general purpose computer which can be part of a network, and also can be connected to a broader network such as the Internet, e.g., for data retrieval. Optionally, the program instruction is installed on a computer system in a manner such that the program instruction can be assessed over a network, e.g., over the Internet. Also optionally, the program instruction or a necessary part thereof may be downloaded from a remote computer over the network, or alternatively may be used for analysis with all or most of the functionality remaining on a storage computer or server. In the latter mode, analysis results can be transmitted to the remote computer.

Examples of program instructions include both machine codes, such as produced by a compiler, and files containing higher level code that may be executed by the computer using an interpreter. Further, the program instructions include machine code, source code and any other code that directly or indirectly controls operation of a computing machine in accordance with this invention. The code may specify input, output, calculations, conditionals, branches, iterative loops, etc.

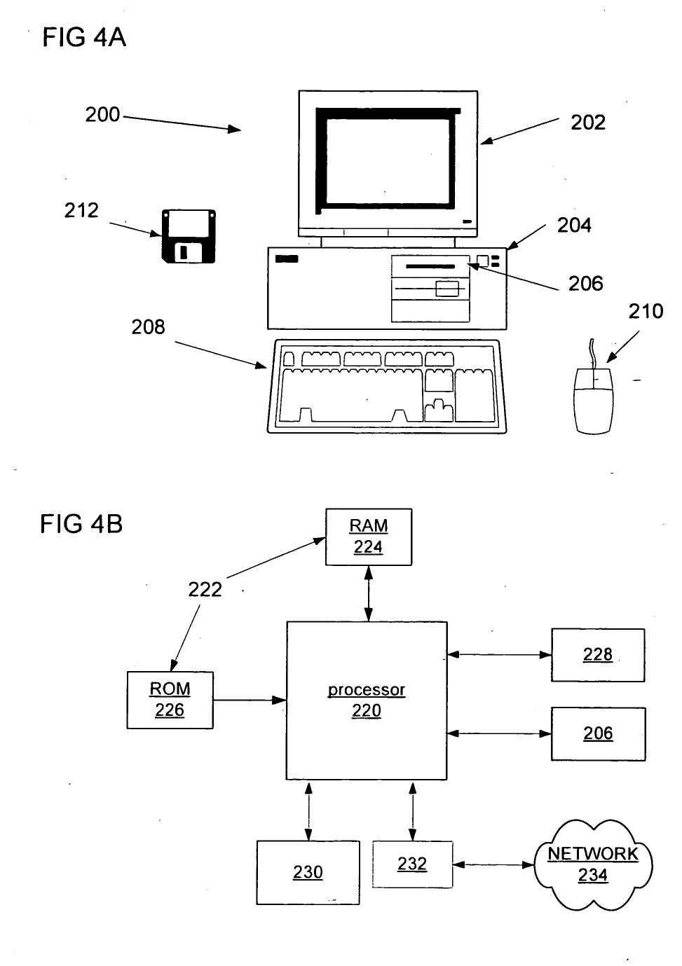

FIGS. 4A and 4B illustrate a computer system 200 suitable for implementing embodiments of the present invention. FIG. 4A shows one possible physical form of the computer system. Of course, the computer system may have many physical forms ranging from an integrated circuit, a printed circuit board and a small handheld device up to a very large supercomputer. Computer system 200 includes a monitor 202, a housing 204, a disk drive 206, a keyboard 208 and a mouse 210. Disk 212 is one example of a computer-readable medium used to transfer data to and from computer system 200.

FIG. 4B is a block diagram of certain logical components of computer system 200. Processor(s) 220 (also referred to as central processing units, or CPUs) are coupled to storage devices including memory 222. Memory 222 includes random access memory (RAM) 224 and read-only memory (ROM) 226. ROM acts to transfer data and instructions uni-directionally to the CPU and RAM is used typically to transfer data and instructions in a bi-directional manner. Both of these types of memories may include any suitable computer-readable medium, including those described above. A fixed disk 228 is also coupled bi-directionally to CPU 220; it provides additional data storage capacity and may also include any of the computer-readable media described below. Fixed disk 228 may be used to store programs, data and the like and is typically a secondary storage medium (such as a hard disk) that is slower than primary storage. It will be appreciated that the information retained within fixed disk 228, may, in appropriate cases, be incorporated in standard fashion as virtual memory in memory 222. Removable disk 206 may take the form of any of the computer-readable media described below.

CPU 220 is also coupled to a variety of input/output devices 230 such as display 202, keyboard 208, mouse 210 and speakers. In general, an input/output device may be any of: video displays, track balls, mice, keyboards, microphones, touch-sensitive displays, transducer card readers, magnetic or paper tape readers, tablets, styluses, voice or handwriting recognizers, biometrics readers, or other computers. CPU 220 optionally may be coupled to another computer or telecommunications network 234 using network interface 232. With such a network interface, it is contemplated that the CPU might receive information from the network, or might output information to the network in the course of performing the above-described method steps. Furthermore, method embodiments of the present invention may execute solely upon CPU 220 or may execute over a network such as the Internet in conjunction with a remote CPU that shares a portion of the processing.

For example, SNP or sequence data for analysis may be recorded in storage media or may be retrieved from a remote location or locations. Typically, such data would be at least temporarily stored in the computer system. Also at least temporarily stored in the computer system will be at least portion of the program instruction of the present invention so that at least a portion of the analysis according to the invention can be performed using the sequence data.

Depending on the program structure, the program instructions for the described analysis according to the present invention can be called or loaded in various ways. For example, the program instructions can be called in portions as needed, all of the instructions of an analysis can be loaded at one time, or analysis with one program can be completed and the resulting data stored before a further program is loaded for additional analysis.

1) General Steps of Data Anlysis

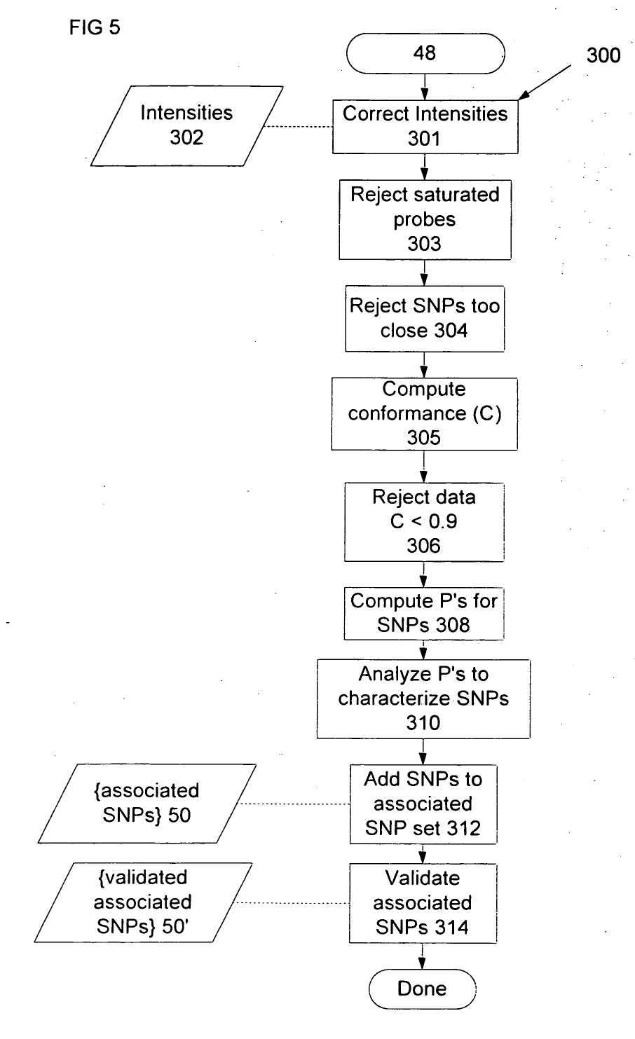

FIG. 5 shows a flowchart illustrating a data analysis method 300 of the invention corresponding to step 48 of the general experimental method 30 shown in FIG. 3. The method 300 includes a number of initial data sanitizing steps, i.e., steps taken to correct the raw intensity data. The computer program implementing the method accesses a stored file or files 302 of raw array fluorescent intensity data for experiments using the control group and case group pooled DNA samples. The intensity data can be indexed by array position or array position data is otherwise associated with the intensity data so that the computer program can determine the site of the arrays from which the intensity data originated. The computer program also has access to data indicating the nature of each probe at each location on the arrays.

A number of intensity correction routines 301 can be carried out on the raw intensity data. Background intensity can inflate intensity measurements causing them to be higher than they should be based on the amount of sample bound to a probe on an array. Background intensity can be dependent or independent of the concentration of sample (target) applied to an array. Concentration-dependent background increases with increasing amounts of sample, and may represent, for example, mismatch and nonspecific hybridization of the sample to the array. Concentration-independent background does not increase with increasing amounts of sample, and can be the result of, for example, scattered or reflected light during scanning of the array. In one embodiment, a background intensity can be subtracted from all of the intensity measurements. In one embodiment, the value of the background intensity is set at the intensity of a probe cell that ranks the 1000^{th} dimmest on the oligonucleotide array. The measured intensity for that probe cell is subtracted from all the intensities as a background correction. Intensity data that are equal to or less than zero after the background correction may be discarded from further evaluation. Of course, other measures could also be used to provide a background intensity value, for example, the 500^{th}, dimmest probe cell or some percent (0.1-3%, for example) of a saturated probe cell.

In an embodiment in which the case and control samples are differently marked or labeled, then the method may correct for differences in detected intensity which are marker-dependent. For example, as noted above, it has been found that there is no linear relationship between the measured intensities for labeling with cychrome and fluorescein. A quadratic fit to measured cychrome and fluorescein intensity data can be carried out to provide a quadratic correction to the cychrome intensity data. The measured cychrome intensity data can then be subjected to this quadratic correction. At high cychrome intensities a parabolic correction function can be used. The correction can be scanner dependent and so different correction data may be used for intensity data collected from different scanners. The background correction described above can be carried out before or after any label-related corrections to the data have been carried out.

In one embodiment, at a data processing step 303, saturated probe cells can also be discarded from further evaluation. If the photomultiplier tube of the scanner measures a saturated probe cell, then the measured intensity is not proportional to the amount of the sample present at the probe cell. Therefore, data collected from the scanner during hybridization and measurement of the raw intensity data, or the intensity data reaching a maximum threshold, can be used to determine if the intensity is in fact a saturated intensity in which case the intensity can be discarded from further evaluation.

In one embodiment, intensity data for SNP positions that are physically too close to each other can be discarded at step 304. If two SNP positions are sufficiently close that a set of probes for a first genotype including one of the two SNPs would also detect a second genotype including the other of the two SNPs, then the intensity data for that set of probes can be discarded from further evaluation.

In a next data processing step 305, a conformance value, C, is calculated for the remaining intensity data, which provides an indication of the reliability of data and so can be used to remove unreliable data from the data set used for subsequent analysis. In general terms, the conformance calculation process 305 involves computing the conformance for each and every SNP on each of the arrays in the set of arrays for the experiment. The conformance calculation uses the non-discarded and corrected measured light intensity data 302 from the hybridized arrays. The conformance calculation process 305 will be described in greater detail below. After conformance values have been calculated for all the SNPs, the conformance data is thresholded 306 and the data for those SNPs having a conformance less than the threshold value are rejected from the data set used in the subsequent steps of the method. In an embodiment, the conformance threshold can be greater than approximately 0.8 and can be approximately 0.9 or greater.

In an additional data processing step (see FIG. 26), a ratio of the amplitude of signal to the amplitude of background, AS/AB, is calculated for the remaining intensity data, which provides an indication of the reliability of data and so can be used to remove unreliable data from the data set used for subsequent analysis. In general terms, the AS/AB calculation process involves computing the ratio of the amplitude of signal to the amplitude of background for each and every SNP on each of the arrays in the set of arrays for the experiment. The AS/AB calculation uses the non-discarded and corrected measured light intensity data from the hybridized arrays. The AS/AB calculation process is shown in FIG. 26 and will be described in greater detail below. After AS/AB values have been calculated for all the SNPs, the AS/AB data is thresholded and the data for those SNPs having a AS/AB value less than the threshold value are rejected from the data set used in the subsequent steps of the method. In one embodiment, the AS/AB threshold can be approximately 1.0 or greater, preferably 1.5 or greater.

A process is then carried out at step 308 to determine an estimate of the relative allele frequency, P′, for each SNP position using the sanitized and conforming intensity data, as will be described in greater detail below. The estimate of relative allele frequency, P′, is computed for each SNP position for each of the experiments performed on case and control groups to provide a set of P′ values for each SNP position for the case and control groups, respectively. The P′ values generally indicate amount of target that has hybridized to the probe(s) for a particular SNP. Generally, if the case or control group has a higher proportion of individuals with a particular SNP, the corresponding feature on an array will be brighter (i.e., will have a higher intensity).

The members of the sets of P′, are then analyzed 310 for each SNP position to determine whether the SNP position can be considered likely to be associated with the disease. If it is determined 310 that the analysis of the P′ data indicates that the SNP position can be characterized as associated with the disease, then the SNP position is added 312 to the set of SNP positions 50 characterized as associated with the disease. If it is determined 310 that the SNP position is not likely to be associated with the disease then the SNP position can be discarded, or otherwise flagged as not, or unlikely to be, associated. This is process is carried out so that all of the SNP positions have been evaluated.

An optional association validation step 314 can be used. The validation step 314, includes applying a secondary test to the set of associated SNP positions 50 to determine whether the characterization as “associated” is consistent with a second criterion. In a preferred embodiment the secondary criterion is based on genetic and/or biological information. This provides a set of validated associated SNPs 50′, in which the validity of the association has been further supported.

Hence the result of the process 300 is a set of associated SNP positions 50, or optionally 50′, that are characterized as likely to be associated with the disease. This information can then be fed back at step 52 into the general association study method 30 as indicated in FIG. 3. This associated SNP information can be used in the selection of SNPs to be used in a newly designed array to remove those SNPs not associated with the phenotype being studied, thereby reducing the amount of data to be collected and/or allowing new SNPs to be added to the arrays to obtain further information relating to the genotype for the case group, as described above.

The results of the above analysis can be output, stored, and/or transmitted to any of a variety of programs, or other functional units further use or preservation.

Various parts of the method will now be described in greater detail. Although the preceding and following flow charts illustrate various operations being carried out together or in a particular sequence, the flow charts are merely intended to be illustrative and not to limit the invention only to the specific order and combinations of operations and processes described. It will be appreciated that various combinations and/or sequences of operations and processes, including omitting various process steps, can be used to put the general methods and processes described herein into effect.

2) Tiling of Probes for a Target SNP

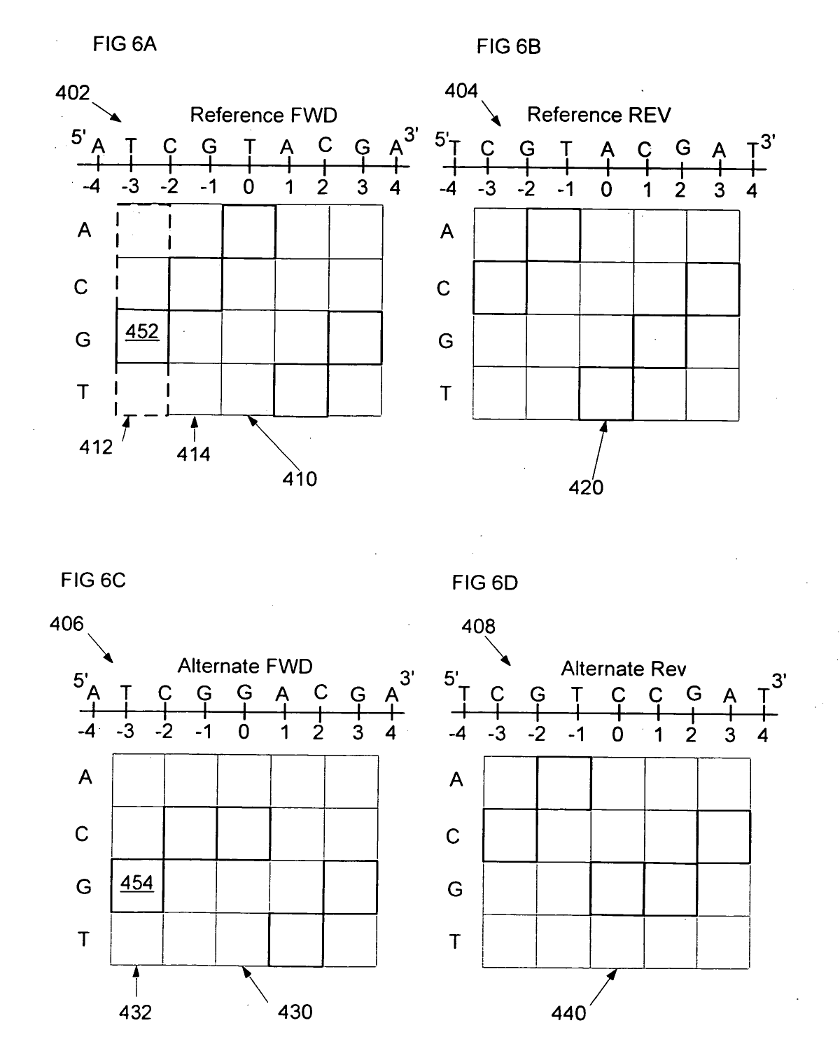

In the illustrated embodiment of the invention, for each particular SNP position of interest, or “target” SNP to be interrogated, a set of eighty probes (referred to as a “tiling” of probes) is provided on an array or distributed over one or more physical arrays. In other embodiments, a tiling of probes may comprise a set of less than eighty probes, for example, 40, 24, or fewer probes. FIGS. 6A, 6B, 6C and 6D respectively show probe tilings 410, 420, 430, 440 of an array associated with a target SNP position. The target SNP position has associated with it a forward reference sequence 402. The numbering of the nucleotides along the sequence indicates the position of a nucleotide relative to the SNP position which is indicated by position 0. The target SNP also has a reverse reference sequence 404 associated with it, which is complementary to the forward reference sequence. The allele at the SNP position for the forward reference sequence is T.

The target SNP also has a forward alternate sequence 406 associated with it, including the alternate allele, G, at the SNP position, and a reverse alternate sequence 408 complementary to the forward alternate sequence. In FIGS. 6A-6D, the sequences 402, 404, 406 and 408 are displayed with the 5′ terminus to the left and the 3′ terminus to the right. As explained above, the two alleles (reference and alternate) for the particular SNP of interest have been determined by experiment and the relevant data is available from, for example, the U.C. Santa Cruz Human Genome Browser Gateway or the NCBI dbSNP website.

Each of the reference forward, reference reverse, alternate forward and alternate reverse sequences has a probe tiling associated with it, 410, 420, 430 and 440 respectively, represented as an array of squares in FIGS. 6A-6D, with each square representing a probe. Each tiling 410, 420, 430 and 440 includes twenty probes and so the tiling for the target SNP has a total of eighty probes. Although the probes are shown as being arranged contiguously and adjacently, it will be appreciated that in practice the probes can be dispersed provided that the position of each probe on the physical array is known. Each column of probes, e.g. column 412, corresponds to a probe set, comprising four probes designed to interrogate a single nucleotide position, and each 20 probe tiling includes five probe sets. Each probe comprises a twenty-five base long nucleotide sequence with the thirteenth position as the interrogation position. Within each probe set, the interrogation position for each probe is substituted by a one of A, C, G or T as indicated in FIGS. 6A-6D. A first probe set is used to interrogate the −2 position of the corresponding sequence, a second probe set the −1 position, the third probe set the SNP position (0), the fourth probe set the +1 position and the fifth probe set the +2 position. The probes for each tiling 410, 420, 430 and 440 are complementary, according to the base pairing rules, to the reference forward and reverse sequences and alternate forward and reverse sequences that they are respectively intended to interrogate.

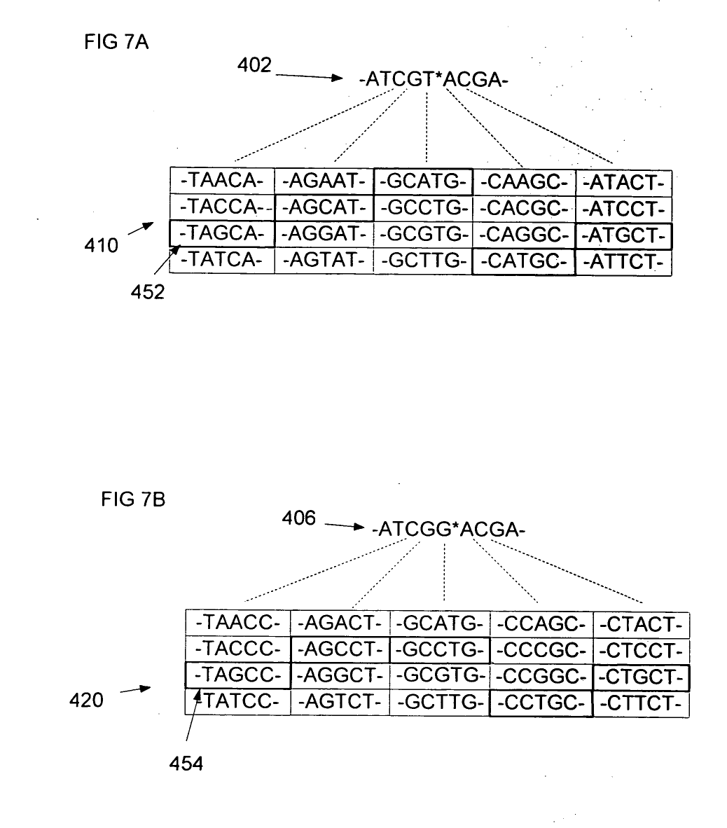

FIGS. 7A and 7B show the sequences of the middle five nucleotides for the forward reference tiling 410 and the forward alternate tiling 420 with the 13^{th} position (interrogation position) in the center. In FIGS. 7A and 7B, the SNP position in the reference allele forward 402 sequence and the alternate allele forward 406 sequence is indicated by an asterisk. As can be seen, for each probe set (e.g., 412), the nucleotide at the interrogation position (the 13^{th} position) is either A, C, G or T. The order is not relevant, provided it is know which probe is at which physical position within the probe set. The four probes of the first probe set are complementary to the target SNP sequence at all positions except the interrogation position, at which the −2 position of the target SNP sequence is interrogated. The actual probes for the reverse sequence tilings 420, 440 have not been shown in FIG. 7, but will be apparent to a person of ordinary skill in the art in view of this description, as will the probe tilings required for the sequences of different target SNPs.

The probes tiling the array and their positions on the arrays are known and have been designed specifically for the reference allele sequences and alternate allele sequences at step 36 of the experimental protocol. As can be seen eighty probes in total are used for each target SNP. (Twenty probes each for the reference allele forward sequence, reference allele reverse sequence, alternate allele forward sequence and alternate allele reverse sequence.)

In the following, ‘tiling’ will indicate, depending on the context, either the twenty probes for a reference allele forward 410, reference allele reverse 420, alternate allele forward 430 or alternate allele reverse 440 sequence individually, or all eighty probes associated with the target SNP together.

Although a twenty-five nucleotide probe sequence has been described above, it will be appreciated that other probe nucleotide lengths and probe-formats can be used.

If conformance data is not required, as will be described further below, then single probes which are perfectly complementary to the respective reference forward, reference reverse, alternate forward and alternate reverse sequences could be used for each tiling 410, 420, 430, 440 in which case four probes could be used. Fewer or more than the five probe sets per forward and reverse sequence of the reference and alternate alleles can be used, but five probes sets has been found to provide reliable conformance data as will be described below.

3) Conformance Assessment

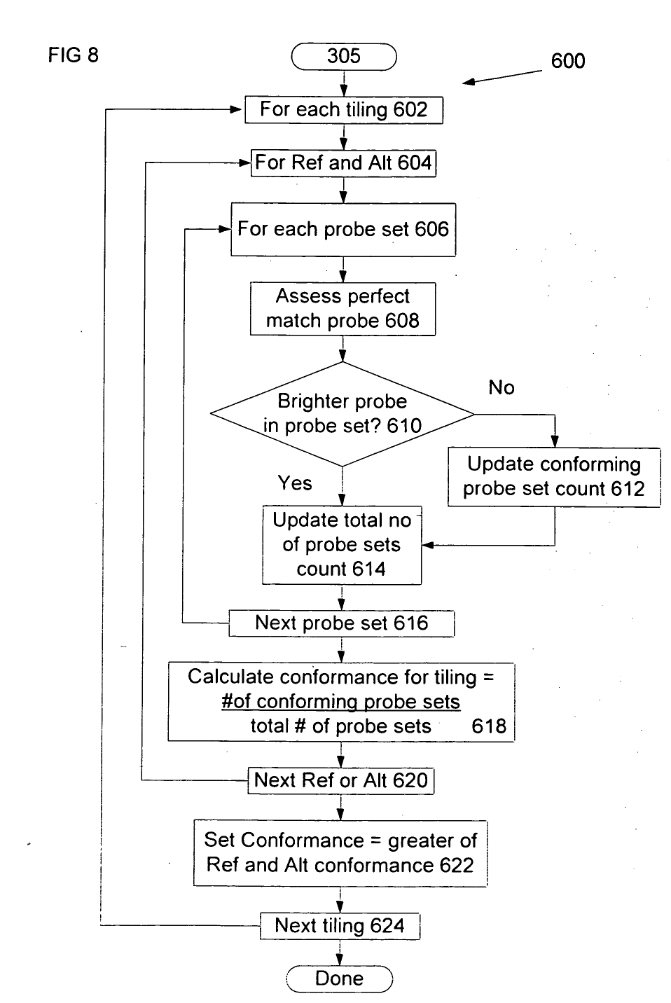

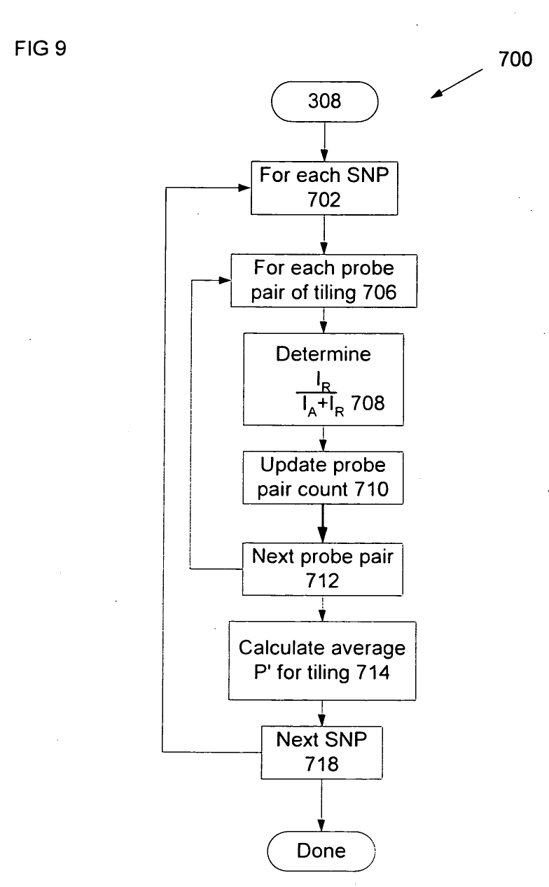

FIG. 8 shows a flow chart illustrating a process 600 by which the reliability of data may be assessed so that, for example, unreliable experimental data can be identified for rejection. FIG. 8 describes in greater detail the operations involved in carrying out the conformance assessment process 305 as shown in FIG. 5. As illustrated in FIG. 5, the data assessment procedure is carried out for all the non-rejected target SNP positions on all the arrays and for both the control and case groups used in the association study, and for each experiment and any replicate experiments. FIG. 8 describes the data assessment process with reference to multiple eighty probe tilings.

A first tiling of the multiplicity of eighty probe tilings is selected at step 602 and the intensity data measured from the probes of the first tiling is used in the conformance assessment. The intensity data for the reference or alternate allele is selected 604 and the intensity data for a first probe set, e.g. probe set 412 as indicated by the broken bold line in FIG. 6A, for the reference sequence is evaluated. The first probe set 412 interrogates the −2 position of the reference allele forward sequence 402. The location of the complementary probe which perfectly matches the reference sequence is looked up from stored data and the measured intensity or brightness for the perfect match probe location is determined 608 from the intensity data 302. As illustrated in FIGS. 6A and 7A, the perfectly matching probe 452 is at the third row of the first probe set. The perfect match probe intensity is then compared 610 with the intensity data for the remaining probes in the probe set, to see if the measured intensity of any of the other probes in the same probe set is brighter, indicating that more DNA had bound thereto. In FIGS. 6A, 6B, 6C and 6D, and associated FIGS. 7A and 7B, the emboldened probes indicate the perfect match probes in each probe set.

If the perfect match probe is determined at step 610 by comparison with the recorded intensities of the other probes in the probe set to be the brightest probe in the probe set, then this is an indication that the probe has bound DNA having the intended sequence. Therefore that probe set can be considered to be conforming, i.e., to include useful experimental data and to be reliable. Therefore a count of the number of conforming probe sets for the reference tilings 410, 420 is updated 612. The process then updates a count 614 of the total number of probe sets for the tilings that have had their conformance evaluated. As can be seen the count of the total number of probe sets evaluated for the reference tilings is updated irrespective of whether a particular probe set conforms or not. If a perfect match probe is not the brightest, then that does not necessarily mean that all the data for the tiling is unreliable, e.g. because the probes are damaged or because there has been some other failure.

The process is then repeated for the next probe set 414 in the tiling 410 for the reference forward and reverse sequences and so on until all ten probe sets for the reference forward and reverse tiling have been evaluated. Then, the conformance for the reference sequence tiling 410, 420 is calculated in step 618. In general, the conformance, C, for a tiling is the number of conforming probe sets divided by the total number of probe sets in the tiling, which in this example is ten.

Hence a conformance for the reference tiling, C_{R}, has been calculated. The process is then repeated 620 using the intensity data for the alternate forward and reverse sequences and a conformance for the alternate sequence, C_{A}, is calculated. The conformance C for the target SNP is then set 622 as the greater of C_{A} and C_{R} to reflect that although the data for the alternate or reference sequence may not be reliable, the data for the other may be reliable and so the conformance value for the better data is used as the metric by which to assess the validity of the data for the target SNP. The conformance value C is stored and indexed by the target SNP and an identifier of whether the data corresponds to the control group or case group, and which experiment. The conformance for a next target SNP is then calculated using the data for the corresponding tiling 624 and the process is repeated until the intensity data for all the 80 probe tilings has been evaluated.

In general, conformance measures the number of times that the presence of one of the reference or alternate sequence is detected out of the number of times either was present. In other words, conformance measures the number of times that the probe that is a perfect match to the sample is the brightest feature (probe) for a given offset, indicating that it hybridized to the sample better than the other features for that offset. It has been found that conformance values below about 0.8 tend to be an indicator that the tiling did not reliably detect the intended nucleotide sequence in the sample. A conformance threshold of 0.8 or greater can be used, but optionally a conformance threshold of 0.9 is used as an indication that either the reference or alternate allele sequence was reliably detected in the DNA sample.

The process is carried out for all of the non-rejected intensity data for all of the experiments to provide a data set of conformance values for each target SNP for each experiment using the control and case group DNA samples. At step 306 of the general method shown in FIG. 5, the conformance values are thresholded and the data for those target SNPs having a conformance below the threshold conformance value of 0.9, optionally below the threshold conformance value of 0.8, optionally below the threshold conformance value of 0.6, are discarded or rejected as likely being unreliable data. This helps to improve the accuracy of the method by removing data from subsequent analysis which is believed not to correspond to a reliable detection of the intended DNA sequence. The use of a conformance metric to identify un-reliable data is not necessary but can improve the accuracy of the method. Although the thresholding and data rejection step 306 has been shown as being carried out after the conformance data has been calculated for all target SNPs and all experiments, it will be appreciated that the thresholding and data rejection step can be carried out at any stage after the conformance has been determined 622 for a target SNP. The reduced intensity data set with the non-conforming data removed, or flagged as unreliable, is then used in the remainder of the method.

4) AS/AB Assessment

FIG. 26 shows a flow chart illustrating another process by which the reliability of data may be assessed so that, for example, unreliable experimental data can be identified for rejection. “Amplitude of Signal” (AS) & “Amplitude of Background” (AB) can be defined so that an acceptable AS to AB ratio (i.e. a “cut-off” value) can determine whether intensity data can be considered reliable for use in calculating P′ values. With reference to FIG. 5, this assessment may be performed before (between steps 304 and 305) or after (between steps 306 and 308) conformance is calculated. The AS/AB ratio assessment is discussed in greater detail below.

5) Calculation of Relative Allele Frequency

FIG. 9 shows a flow chart illustrating a process 700 for carrying out step 308 of the general method in greater detail. The method of the present invention takes into account the fact that either the reference allele for the SNP position of interest, or the alternate allele for the SNP position of interest can be present in the DNA of a person in the case group or control group. Either the reference allele or the alternate allele may be involved in the disease mechanism, and it may be a combination of reference and alternate alleles at various SNP positions which results in the manifestation of the phenotypic trait of interest. Using a group of individuals to provide pooled case and control samples means that a measure of the frequency of occurrence of the reference or alternate allele at the SNP position in the case population and the control population can be obtained from the intensity measurements.

The relative allele frequency, P, for a SNP position indicates the proportion of the reference and alternate alleles at the SNP position. P, in one specific embodiment, could be calculated from the concentration of the reference allele (cᵣ) at a SNP position divided by the total concentration of the alternate and reference alleles at the SNP position (cₐ+cᵣ), i.e. P=cᵣ/(cₐ+cᵣ). For example, in the scenario illustrated in FIG. 2, if x indicates the alternate allele, then for SNP position 1, P=5/6 for the control group and P=4/6 for the case group. However, it is not possible in practice to measure the actual allele concentrations precisely. In one specific embodiment, a factor related to concentration is utilized. In the case of using a DNA probe array, in one specific embodiment, the intensity of the emitted light from the probe sites is measured and is related to the concentration at that probe site. Therefore an estimator, P′, of the relative allele frequency can be calculated from the measured experimental data and in general can be calculated using the intensity of light from the reference allele sequence (I_{R}) probe as a proportion of the total intensity of light from the alternate and reference allele sequence probes (I_{A}+I_{R}), i.e. P′=I_{R}/(I_{A}+I_{R}). The use of I_{R}/(I_{A}+I_{R}) rather than I_{A}/(I_{A}+I_{R}) is merely a matter of convention and the latter could be used in the alternate as an estimated measure of the relative allele frequency defined by cₐ/(cₐ+cᵣ). General equations are in for illustration only; other factors may be multiplied by and/or added to the equations.

P′ has been found to have a relationship to P and, while in some circumstances it can be approximately linear, P′ typically has a higher order relationship to P. However, in general if P′ is determined for a particular SNP position for two different pools of the same nucleic acid sample, then P′ is found to vary by a small amount the majority of the time. Therefore, although the relationship between P and P′ may vary, calculations based on P′ can be robust. In particular, it has been found that the relationship between P and P′ may vary between production runs of physical arrays. Therefore it is preferable that the present method use data collected from arrays of the same production batch. However, the methods may also be used for data collected from arrays of different production batches.

As illustrated in FIG. 9, the process 700 of computing P′ values, corresponding to general method step 308, initially uses the intensity data for a first target SNP position 702 from the target SNP positions whose data has not been rejected, e.g., after the conformance test and/or AS/AB assessment. The data for the forward and reverse sequences can be processed separately, but in the illustrated embodiment the forward and reverse data are combined. The intensity data for the pair of perfectly matching probes at a particular offset from the first probe set for the reference and alternate sequences is identified at step 706. I_{R}/(I_{A}+I_{R}) is calculated at step 708 using the intensities for the matching probe position 452 for the first probe set 412 for the reference allele and the matching probe position 454 for the first probe set 432 for the alternate allele (represented by emboldened outlines in FIGS. 6A, 6B, 6C, 6D, 7A and 7B).

A probe pair count is incremented at step 710 and then the process is repeated at step 712 for each of the pairs of perfectly complementary probes in the tilings 410, 420 for the current target SNP position. When all of the probe pairs have been evaluated, an average value for P′ for the target SNP position is calculated 714 at step as the sum of the relative intensities for each probe pair divided by the total number of probe sets, which in this example is ten. This formula provides P′ for this target SNP position. This P′ value indicates the relative allele frequency for the population of the case or control groups, as the DNA samples for the case and control groups were each pooled. Hence the P′ value is that for the target SNP position averaged over the population of either the case or control groups. The averaged P′ value is stored. At step 718, the process is repeated for the next target SNP position until average P′ values have been calculated for all the target SNP positions using the stored intensity data.

The P′ value generally indicates the relative amount of target that has hybridized to the reference and alternate perfect match probe(s) for a particular SNP. In general, if the case or control group has a higher proportion of individuals with a particular SNP allele, the corresponding features on any array will be brighter. The difference in relative allelic frequency (ΔP′) is equal to the relative reference allelic frequency in a case group minus the relative reference allelic frequency in a control group, i.e. ΔP′=P′_{case}−P′_{control}. Where P′ is calculated based on the proportion of the reference allele relative to the alternate allele, a positive difference in relative allelic frequency (i.e. a positive ΔP′ value) indicates that the reference variant is associated with the case group and the alternate variant is associated with the control group; and a negative difference in relative allelic frequency (i.e. a negative ΔP′ value) indicates that the alternate variant is associated with the case group and the reference allele is associated with the control group.

6) Methods for Calculating P′

The computation of the estimate of relative allele frequency, P′, initially uses the intensity data for a first target SNP position from the target SNP positions whose data has not been rejected, for example, after the conformance test or the AS/AB assessment. For each particular SNP position of interest, or “target” SNP, to be interrogated, a set of eighty probes (referred to as a “tiling” of probes) may be provided, which include probes to interrogate at offsets −2, −1, 0, 1, 2 for both forward and reverse sequences for reference and alternate alleles. In other words, an eighty probe tiling may contain 20 probes for each of the “reference forward”, “reference reverse”, “alternate forward” and “alternate reverse” tilings, as shown in FIG. 6. A first example of a method for calculating P′ involves determining P′ᵢ values at a plurality of different offsets, and then averaging those P′ᵢ values. At each offset, there is one pair of perfectly matching probes (one reference and one alternate) for the forward tiling and one pair of perfectly matching probes (one reference and one alternate) for the reverse tiling. So, there are a total of 5 pairs of perfectly matching probes for the forward tiling 5 perfectly matching probes for the reverse tiling. The perfectly matching probes are outlined with bold lines in FIG. 6. For the first probe set, the intensity data for the pair of perfectly matching probes at a particular offset (for example, probes 452 and 454 at offset −2 in FIG. 6) is identified and P′ᵢ is calculated with the formula I_{R}/(I_{A}+I_{R}), where I_{R} is the intensity measurement for the perfectly matching reference probe (e.g. 452) and I_{A} is the intensity measurement for the perfectly matching alternate probe (e.g. 454). The process is repeated at each offset for probes in the forward and reverse tilings for the current target SNP position. When all of the perfectly matching probe pairs have been evaluated, an average value for P′ for the target SNP position is calculated.

In some embodiments, the P′ value can be calculated by taking an average of the intensity ratios (i.e., “Mean of the Intensity Ratios”), which is the sum of the relative intensities for each perfect match probe pair P′ᵢ divided by the total number of perfectly matching probe pairs. The following formula can be used for calculating P′ for this target SNP position:

$${P}^{′} = 〈 {P}_{i}^{′} 〉 = \frac{{\sum }_{i = 1}^{n} {P}_{i}^{′}}{n} = \frac{{\sum }_{i = 1}^{n} ( \frac{{I}_{R}}{{I}_{A} + {I}_{{R}_{i}}} )}{n} = 〈 {I}_{R} / ( {I}_{A} + {I}_{R} ) 〉 ,$$

where n=total number of the offsets.

The P′ value calculated using this formula may be intensity- and/or signal-dependent, for example sensitive to the “outliers”, which are data points outside the normal intensity range. Outlier may be due to “speckles”, which may cause a hybridization-independent increase in probe intensity. Outliers may also arise where, while I_{R} and I_{A} may not be individually outside the normal intensity range, I_{R} is at the high end of the normal range and I_{A} Is at the low end causing the ratio I_{R}/I_{R}+I_{A} to be higher than it should be.

In other embodiments, the P′ value can be calculated by averaging the perfect match intensity measurements at a plurality of different offsets, then determining P′ by calculating a ratio of those average intensity measurements (i.e. “Ratio of the Mean Intensities”), which reduces the sensitivity of the P′ calculation to outliers:

$${P}^{′} = \frac{〈 {I}_{R} 〉}{〈 {I}_{A} 〉 + 〈 {I}_{R} 〉}$$

In some embodiments, 24 perfect match probes (comprising 12 perfectly matching probe pairs) may be used to calculate P′ values since there are four additional probes in the 80 probe tiling that are perfectly complementary to the reference or alternate sequence, considering that at position 0 (or offset 0) there is a perfect match to the reference sequence in the alternate tiling and a perfect match to the alternate sequence in the reference tiling. In other words, at the 0 offset for a particular tiling (reference forward, alternate forward, reference reverse, or alternate reverse), there are actually two perfect match probes, one that is complementary to the reference sequence and the other that is complementary to the alternate sequence. This is because at the 0 offset the tilings are duplicated between the reference and alternate tilings for a given strand orientation (forward or reverse). For example, in the reference forward tiling in FIG. 6 the top probe at the 0 offset is perfectly complementary to the reference sequence and the probe below the top probe is perfectly complementary to the alternate sequence. Therefore, the intensity measurements of the four additional perfect match probes (two additional perfect match probe pairs) may also be included in the P′ calculation for a given SNP, regardless of whether the “Mean of the Intensity Ratios” or the “Ratio of the Mean Intensities” method is used.

In further embodiments, rather than an arithmetic mean calculation, a trimmed mean may be used to calculate P′ from the P′ᵢ values or the intensity measurements. A trimmed mean is found by ignoring the k smallest observations and the k largest observations and averaging the rest of the sample, and is discussed in greater detail below. In still further embodiments, a Winsorized mean may be used to calculate P′ from the P′ᵢ values or the intensity measurements. A Winsorized mean is similar to a trimmed mean except that instead of ignoring the k smallest and k largest observations, those observations are replaced by the nearest non-extreme values. For example, if k=2 the smallest and 2^{nd} smallest values are each replaced by the 3^{rd} smallest value, and the largest and 2^{nd} largest values are each replaced by the 3^{rd} largest value.

In other embodiments, a background correction may be subtracted from the individual intensity measurements prior to calculating P′ for a given SNP. Determination of values for a background correction are discussed in detail below.

As one of skill will readily recognize, these methods may be combined in different ways. For example, one may use the “Mean of the Intensity Ratios” method with 12 perfect match probes and a background correction, or one may use the “Ratio of the Mean Intensities” method with 10 perfect match probes and no background correction. Further, either method may use trimmed, Winsorized or arithmetic means to calculate P′. One particular embodiment is described below.

6) One Example of a Method for Calculating P′—.“Ratio of the Mean Intensities”

The P′ value may be calculated in a different way to reduce its sensitivity to the outliers as follows. This method involves averaging the intensity measurements at a plurality of different offsets, then determining P′ by calculating a ratio of those average intensity measures as shown in the equation below. In this example, the total number of perfectly matching probe pairs used is 12, instead of 10 as in the example described above, although the method may be used for 10 perfectly matching probe pairs, as well. Thus,the P′ value calculated in this example is computed from 12 intensities which represents a 20% increase in the amount of data compared to that calculated based on 10 matching probe sets.

In this example, the P′ value can be calculated by taking the average of the intensities before calculating the ratio so that the P′ value is less sensitive to the “outliers” by using the formula shown below.

$${P}^{′} = \frac{〈 {I}_{R} 〉}{〈 {I}_{A} 〉 + 〈 {I}_{R} 〉}$$

Although an arithmetic mean may be taken, the sensitivity of P′ to the “outliers” can be further reduced by taking a trimmed mean of the intensities. A trimmed mean is found by ignoring the k smallest observations and the k largest observations and averaging the rest of the sample. The number of points excluded at each end of the distribution, k, can be chosen. In this example, there are 6 perfect matches for the forward sequence and 6 for the reverse sequence. Thus, there is a total of 12 for each reference and alternate allele, i.e., 6 each for I_{A,for}, I_{R,for}, I_{A,rev}, and I_{A,rev}. For each of I_{A,for}, I_{R,for}, I_{A,rev}, and I_{R,rev}, the six data points are ordered from smallest to largest.

Some centered fraction of each data set will be used for calculating the intensity averages. In this example, out of the 6 perfect match intensities for each of I_{A,for}, I_{R,for}, I_{A,rev}, and I_{R,rev}, the middle half of the ordered intensities is preferred for the calculation of the trimmed mean of the average intensity; so, for a data set of six points, three data points are desired. However, there is no “middle three” in an ordered set of six, so the middle four points are taken with the outside two points weighted at only ½ of the weight of the middle two points. In other words, the middle interval (values #3 and #4) is extended by one probe on each side (values #2 and #5) and the first (#2) and last (#5) are taken with a weight of ½. (Thus, the “middle three” is ½+1+1+½.) Since not all the data points are weighted the same, this trimmed mean is also a “weighted mean”. Of course, one of skill will readily recognize that the fraction of observations included in the calculation of average intensity may vary and range from, for example, 50% to 95%, depending on, for example, the size of the data set. Shown below is an example of how to calculate a trimmed mean for I_{A,for}, <I_{A,for}>, for the perfect match (PM) probes:

$$〈 {I}_{A , for}^{pm} 〉 = \frac{\frac{1}{2} {I}_{A , for 2}^{pm} + {I}_{A , for 3}^{pm} + {I}_{A , for 4}^{pm} + \frac{1}{2} {I}_{A , for 5}^{pm}}{3}$$

Similarly, <I_{R,for}^{pm}>,<I_{A,rev}^{pm}>, and <I_{R,rev}^{pm}> can be calculated using the same formula substituting the appropriate intensity measures from the “reference, forward”, “alternate reverse”, and “reference reverse” tilings, respectively.

In certain embodiments, a background correction is used to further analyze and process the intensity data prior to calculating P′. This may be performed by subtracting a background correction from each intensity measurement as discussed above. In other embodiments, a background correction for the average intensities can be calculated. For instance, when applying only reference sequence to an array, a “perfect” concentration-dependent and concentration-independent background subtraction would make the <I_{A}>=0 (since no alternate sequence is being applied) while <I_{R}>≠0. Since the difference between the reference and alternate sequence is at one nucleotide position (i.e., is one “mismatch”), the intensity of a probe containing one mismatch to the sequence applied would be an ideal background correction. As such, the concentration-dependent and concentration-independent background derived from mismatch probes can be subtracted from perfect match probes, and in order to have the <I_{A}>=0 for the reference sequence, one needs to subtract as background the intensity of one mismatch. Since all the intensity data must be treated the same, this correction would be subtracted from both <I_{R}> and <I_{A}> measures. However, since the identity of the target sequence being applied to the array is not known in advance, the probes that represent only one mismatch are also not known. Therefore, as an approximation of the intensity of a single mismatch probe, the intensity of all the mismatch probes are averaged and that value (“background intensity”) is subtracted from <I_{R}> and <I_{A}> measures.

In certain embodiments, the background intensity can be calculated by taking an arithmetic mean of the intensities of mismatch probes, and in other embodiments a Winsorized mean of the intensities of mismatch probes is used. In still other embodiments, such as in the example described in detail below, a trimmed mean of the intensities of mismatch probes is utilized to calculate a background intensity value. As shown in the formula below, the mean intensity of the mismatch probes in the forward reference tiling (<I_{R,for}^{mm}>) is calculated. The mean intensities of mismatch probes in the reverse reference (<I_{R,rev}^{mm}>), forward alternate (<I_{A,for}^{mm}>), and reverse alternate (<I_{A,rev}^{mm}>) tilings may also be calculated in the same manner using the intensities of the respective mismatch probes. According to this formula, the middle half (½×14) in the distribution is preferred. Since 7 points are not evenly distributed in the middle of the ordered set of intensity measurements, 8 points in the middle are taken instead (disregarding the 3 points at each end of the distribution). The 1^{st} and 8^{th} points are weighted by half and the rest of the points are weighted as full values (still a total weight of 7 points) in calculating the mean:

$$〈 {I}_{R , for}^{m m} 〉 = \frac{\begin{matrix}\frac{1}{2} {I}_{{R}_{4} for}^{m m} + {I}_{{R}_{5} for}^{m m} + {I}_{{R}_{6} for}^{m m} + {I}_{{R}_{7} for}^{m m} + \\ {I}_{{R}_{8} for}^{m m} + {I}_{{R}_{9} for}^{m m} + {I}_{{R}_{10} for}^{m m} + \frac{1}{2} {I}_{{R}_{11} for}^{m m}\end{matrix}}{7}$$

The resulting mean intensity data (<I_{R,for}^{mm}>, <I_{R,rev}^{mm}>, <I_{A,for}^{mm}>, and <I_{A,rev}^{mm}>) may then be averaged and the resulting average intensity for the mismatch probes is used as Background in computing the P′ value:

$$\begin{matrix}Background = \frac{〈 {I}_{R}^{m m} 〉 + 〈 {I}_{A}^{m m} 〉}{2} \\ = ( \frac{〈 {I}_{R , for}^{m m} 〉 + 〈 {I}_{R , rev}^{m m} 〉}{2} + \frac{〈 {I}_{A , for}^{m m} 〉 + 〈 {I}_{A , rev}^{m m} 〉}{2} ) / 2 \\ = \frac{〈 {I}_{R , for}^{m m} 〉 + 〈 {I}_{R , rev}^{m m} 〉 + 〈 {I}_{A , for}^{m m} 〉 + 〈 {I}_{A , rev}^{m m} 〉}{4}\end{matrix}$$

Where <I_{R,for}^{mm}>, <I_{R,rev}^{mm}>, <I_{A,for}^{mm}>, and <I_{A,rev}^{mm}> are trimmed means.
Although trimmed means are used for the calculation of <I_{R,for}^{mm}>, <I_{R,rev}^{mm}>, <I_{A,for}^{mm}>, and <I_{A,rev}^{mm}> in this example, arithmetic or Winsorized means may also be used for these calculations. Further, although the mean intensity data is calculated separately for the four tilings (reference forward and reverse, and alternate forward and reverse) in this example, in other embodiments the intensities for all the mismatch probes for all the tilings may be averaged together in a single arithmetic, trimmed, or Winsorized mean calculation to derive a Background correction.

In still other embodiments, a background correction may be calculated across an entire array. For example, the intensities of all the mismatch probes on an array may be averaged (e.g., trimmed mean, Winsorized mean, or arithmetic mean) to calculate one background correction for all the intensity measurements collected from a single scan.

The computation of P′ value for some embodiments is summarized in the following equations. The intensity data for the perfect match probes for each of the reference and alternate sequences (forward and reverse) are identified and averaged by taking the trimmed mean, from which the average intensities for the perfect match reference and for the perfect match alternate (<I_{R}^{pm}> and <I_{A}^{pm}>) are calculated:

$$〈 {I}_{R}^{pm} 〉 = \frac{〈 {I}_{R , for}^{pm} 〉 + 〈 {I}_{R , rev}^{pm} 〉}{2}$$

$$〈 {I}_{A}^{pm} 〉 = \frac{〈 {I}_{A , for}^{pm} 〉 + 〈 {I}_{A , rev}^{pm} 〉}{2}$$

Where <I_{R,for}^{pm}>, <I_{R,rev}^{pm}>, <I_{A,for}^{pm}>, and <I_{A,rev}^{pm}> are trimmed means.

The average intensity for the reference and alternate (<I_{R}> and <I_{A}>) are calculated by subtracting the Background from the average intensities for the perfect matches, from which the relative intensity, or P′, are calculated:

$${P}^{′} = \frac{( 〈 {I}_{R}^{pm} 〉 - \frac{〈 {I}_{R , for}^{m m} 〉 + 〈 {I}_{R , rev}^{m m} 〉 + 〈 {I}_{A , for}^{m m} 〉 + 〈 {I}_{A , rev}^{m m} 〉}{4} )}{\begin{matrix}( 〈 {I}_{A}^{pm} 〉 - \frac{〈 {I}_{R , for}^{m m} 〉 + 〈 {I}_{R , rev}^{m m} 〉 + 〈 {I}_{A , for}^{m m} 〉 + 〈 {I}_{A , rev}^{m m} 〉}{4} ) + \\ ( 〈 {I}_{R}^{pm} 〉 - \frac{〈 {I}_{R , for}^{m m} 〉 + 〈 {I}_{R , rev}^{m m} 〉 + 〈 {I}_{A , for}^{m m} 〉 + 〈 {I}_{A , rev}^{m m} 〉}{4} )\end{matrix}}$$

or, in another form,

$${P}^{′} = \frac{( 〈 {I}_{R}^{pm} 〉 - \frac{〈 {I}_{R}^{m m} 〉 + 〈 {I}_{A}^{m m} 〉}{2} )}{( 〈 {I}_{A}^{pm} 〉 - \frac{〈 {I}_{R}^{m m} 〉 + 〈 {I}_{A}^{m m} 〉}{2} ) + ( 〈 {I}_{R}^{pm} 〉 - \frac{〈 {I}_{R}^{m m} 〉 + 〈 {I}_{A}^{m m} 〉}{2} )}$$

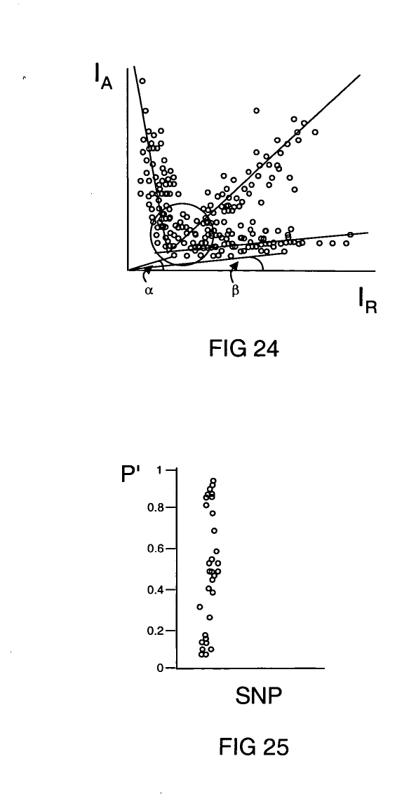

These methods for calculating the P′ value are particularly useful for low intensities, although the example works well with a wide range of intensities. If the P′ values from various diploid samples are plotted on an intensity map, where I_{R} is the X-axis and I_{A} is the Y-axis, the distribution of P′ values can be illustrated in FIG. 24. Ideally, all the reference samples (homozygous) should line up on the x-axis and all the alternate samples (homozygous) should line up on the y-axis, with the heterozygous sample on a line that intersects at zero and has a slope of 1. Thus, a perfect reference sample, a perfect heterozygous sample, and a perfect alternate sample should have a P′ value of 1, 0.5 and 0, respectively. In reality, due to concentration-dependent and concentration-independent background, the reference and alternative samples are clustered and deviated from the axes with a wide spread of background-uncorrected P′ values. For example, two different reference samples at each end of the intensity range can have very different background-uncorrected P′ values, which are represented by the angles α and β (FIG. 24), whereas in theory they should both have a P′ of 1. In some embodiments, since background-corrected P′ can zoom in to the intersect region (circled area in FIG. 24), it can better discriminate the different intensities and represent the data more accurately.

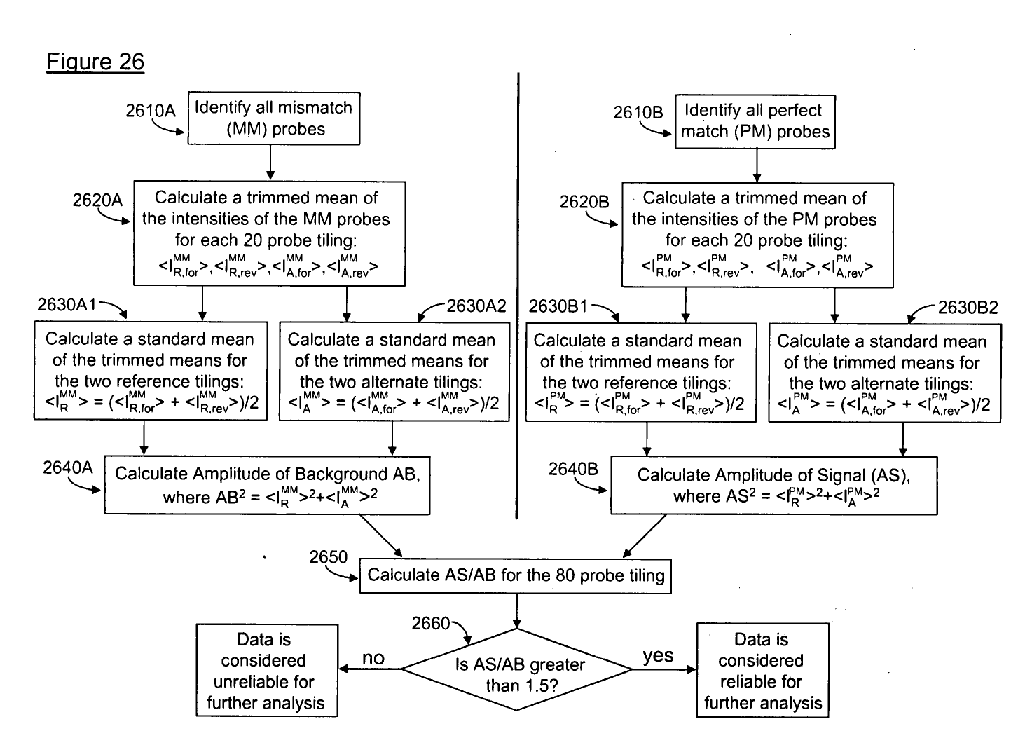

By correcting the concentration-dependent and concentration-independent background, the P′ values are brought closer to what their intrinsic values should be. If the P′ values for various SNPs are plotted on a graph, they are now clustered at around P′=1, 0.5, and 0 (FIG. 25). P′ values calculated without such correction may be scattered anywhere between 0 to 1.

The analysis of P′ or the difference in P′ (ΔP′) works well for both individual genotyping and for pooled genotyping where pool of cases and pool of controls from different individuals are used as the samples.

7) AS/AB Threshold Analysis for Calculation of P′

“Amplitude of Signal” (AS) & “Amplitude of Background” (AB) can be defined so that an acceptable AS to AB ratio (i.e. a “cut-off” value) can determine whether intensity data can be considered reliable for use in calculating P′ values. A flow diagram describing how to calculate an AS/AB ratio is shown in FIG. 26.

The “amplitude of signal” (AS) is defined as the hypotenuse in a right triangle with <I_{R}^{pm}> and <I_{A}^{pm}> being the other two sides of the right triangle, where <I_{R}^{pm}> and <I_{A}^{pm}> are the intensity of perfect match reference probes and the intensity of perfect match alternate probes, respectively. According to the Pythagorean Theorem, which asserts that for a right triangle, the square of the hypotenuse is equal to the sum of the squares of the other two sides: a²+b²=c², the AS can be calculated at step 2640B using:
AS=\sqrt{<I_{R}^{pm}>²+<I_{A}^{pm}>²} (taken from perfect match probes),
Where

$$〈 {I}_{R}^{pm} 〉 = \frac{〈 {I}_{R , for}^{pm} 〉 + 〈 {I}_{R , rev}^{pm} 〉}{2} , 〈 {I}_{A}^{pm} 〉 = \frac{〈 {I}_{A , for}^{pm} 〉 + 〈 {I}_{A , rev}^{pm} 〉}{2} ,$$
calculated at step 2630B1 and step 2630B2, respectively, and
Where <I_{R,for}^{pm}>, <I_{R,rev}^{pm}>, <I_{A,for}^{pm}>, and <I_{A,rev}^{pm}> are trimmed means calculated at step 2620B (although arithmetic or Winsorized means may also be used).

Similarly, the “amplitude of background” (AB) is defined as the hypotenuse in a right triangle with <I_{R}^{mm}> and <I_{A}^{mm}> being the other two sides of the right triangle, where <I_{R}^{mm}> and <I_{A}^{mm}> are the intensity of mismatch reference probes and the intensity of mismatch alternate probes, respectively. According to the Pythagorean Theorem, the AB can be calculated at step 2640A using:
AB=\sqrt{<I_{R}^{mm}>²+<I_{A}^{mm}>²} (taken from mismatched probes)
Where

$$〈 {I}_{R}^{m m} 〉 = \frac{〈 {I}_{R , for}^{m m} 〉 + 〈 {I}_{R , rev}^{m m} 〉}{2} , 〈 {I}_{A}^{m m} 〉 = \frac{〈 {I}_{A , for}^{m m} 〉 + 〈 {I}_{A , rev}^{m m} 〉}{2} ,$$
calculated at step 2630A1 and step 2630A2, respectively, and
Where <I_{R,for}^{mm}>, <I_{R,rev}^{mm}>, <I_{A,for}^{mm}>, and <I_{A,rev}^{mm}> are trimmed means calculated at step 2620A (although arithmetic or Winsorized means may also be used). Finally, the AS/AB ratio is calculated at step 2650.

In various embodiments, different AS/AB ratio thresholds can be chosen. In some embodiments, the AS/AB ratio threshold is determined based on the free energy of association (ΔG) between the target and the probe on the array. Depending on the data and methodology, the threshold value can be any number within the range of 1 to 10. For a particular data set, if the AS/AB ratio is below the threshold value, this data will not be included in further analysis and is a “no pass”. One example is an AS/AB ratio threshold of 1.5 for the quality analysis, as shown in step 2660. The AS/AB ratio is another quality control step in the genotyping experiments and may be performed after the initial conformance assessment of each probe set. An AS/AB threshold may be used to accept or reject data to be used in either the “Mean of the Intensity Ratios” or the “Ratio of the Mean Intensities” P′ calculation.

By using the calculation methods provided herein, sets of P′ data for the control and case groups can be generated for all of the target SNP positions for each experiment. The experiment can be replicated a number of times, using the same DNA samples from the same control and case groups and the same array designs to provide a number of sets of P′ each corresponding to different one of the replicated experiments. For example, step 46 (FIG. 3) can correspond to hybridizing six duplicate sets of arrays with different PCR reactions which provide six sets of experimental results. The P′ values from a single experiment or from a number of experiments can be subjected to a number of different analyses in order to characterize the SNP location as associated with the disease/condition or not. Examples of various methods for analyzing the P′ values, corresponding to step 310 of process 300, will now be described in greater detail below.

One of the aims of the present invention is to identify SNP positions that may be characterized as likely to be associated with the disease or other phenotypic trait exhibited by the members of the case group and not exhibited by members of the control group. The number of SNP positions that are ultimately characterizable as being associated with a phenotype by the methods described herein has been found to occur at the level of a few tens to a few hundred SNP positions per thousands to millions of initial SNP positions. In the following examples, for the purposes of explanation, it is assumed that six replicate experiments were carried out where data from multiple experiments is used.

8) Analysis of P′ Values to Identify Associated SNP Positions

i) Threshold Analysis

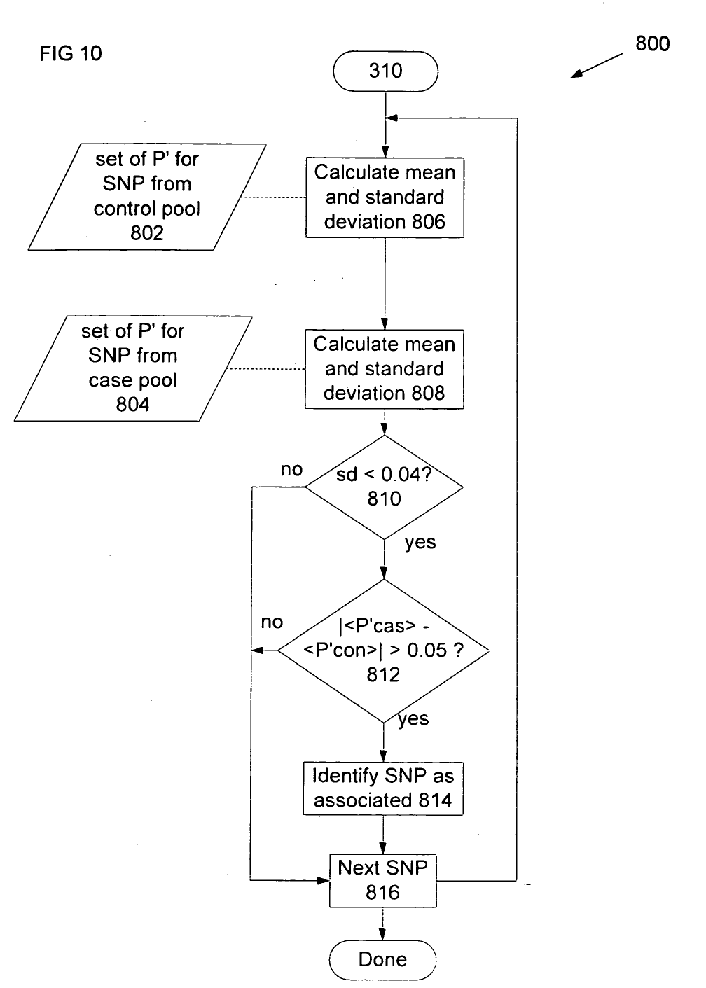

FIG. 10 shows a flow chart illustrating a process 800 for analyzing P′ values to characterize a SNP position as being associated or not. A set of P′ values from the repeated experiments and for the control group for a SNP position 802 is used at step 806 to calculate their mean <P′con> and their standard deviation, σ_{con}. Similarly, a set of P′ values from the repeated experiments and for the case group for the same SNP position 804 is used to calculate at step 808 their mean <P′cas> and their standard deviation, σ_{cas}. Then, each of the standard deviations are thresholded 810 to see if the P′ values can be considered reliable for use in characterizing the SNP position. In one embodiment, a threshold of 0.04 is used. If either the case or control standard deviation does not meet the threshold criterion (e.g., if either the case or control standard deviation is greater than 0.04), then the set of P′ data are rejected and a next SNP is selected at step 816 for evaluation.

In a further embodiment, a variable standard deviation threshold value (cutoff) is used depending on the number of P′ values available to calculate the mean and standard deviation. For a small number of data items, a larger standard deviation may be acceptable and so the cutoff would be increased. However, for a larger number of data items, the standard deviation of the sample can be considered a reasonable estimate of the actual standard deviation of the underlying population and so a more stringent standard deviation cutoff can be used to help identify and reject unreliable results.

In an alternative embodiment, a chi-squared distribution is used to determine a standard deviation cutoff. The chi-squared distribution corresponding to the number of P′ values available and the maximum acceptable true standard deviation for the null hypothesis is used to determine the likelihood of observing as large a sample standard deviation under that null hypothesis. If it is determined that the sample standard deviation is not so large as to be inconsistent with the maximum acceptable standard deviation, then the sample standard deviation is “acceptable” and the thresholding step 810 is met. If the likelihood under this null hypothesis is unacceptably small, then the thresholding step 810 is not passed and the data items are rejected. Using the chi-squared distribution, a confidence level of approximately 95% and a maximum acceptable true standard deviation of 0.01-0.1 could be used to determine whether the calculated standard deviation is acceptable or not.

If both the case and control standard deviations pass the thresholding step 810, then the magnitude of the difference in the mean P′ for the case group and the mean P′ for the control group is calculated, i.e. |<P′cas>−<P′con>|=|Δ<P′>|. As it is the magnitude that is relevant in this embodiment, it will be understood that <P′con>−<P′cas> can be calculated instead. The magnitude of the difference in mean P′s is then thresholded 812. The threshold criterion used can be any criterion which is sufficient to distinguish with an acceptable degree reliability between significant |Δ<P′>|s and those which are unlikely to be significant. In one embodiment a threshold of 0.05 can be used. Optionally, a threshold of between 0.02 and 0.2 may be used. If the threshold is criterion is met, then the difference is deemed sufficient for the SNP position to be considered an associated SNP position, as there can be considered to be a genuine difference in the relative allele frequencies at the SNP position in the case and control groups. Therefore the SNP position is identified at step 814, by a flag or any other suitable mechanism, as being an associated SNP position. A next SNP position is then selected at step 816 for evaluation.

In another embodiment, a first threshold is used during a first iteration of the general method and a second threshold is used during the second iteration for the reduced number of SNPs. The second threshold can be greater than the first threshold. The second threshold can be between 0.05 and 0.5, optionally between 0.07 and 0.3, or optionally between 0.08 and 0.2. In a preferred embodiment a threshold of approximately 0.05 is used during the first iteration and a threshold of 0.10 is used in the second iteration. The first threshold acts as a coarse filter to identify associated SNPs and the second threshold acts as a finer filter so that the characterization of SNPs by the second iteration is more robust.

ii) T-Test Analysis

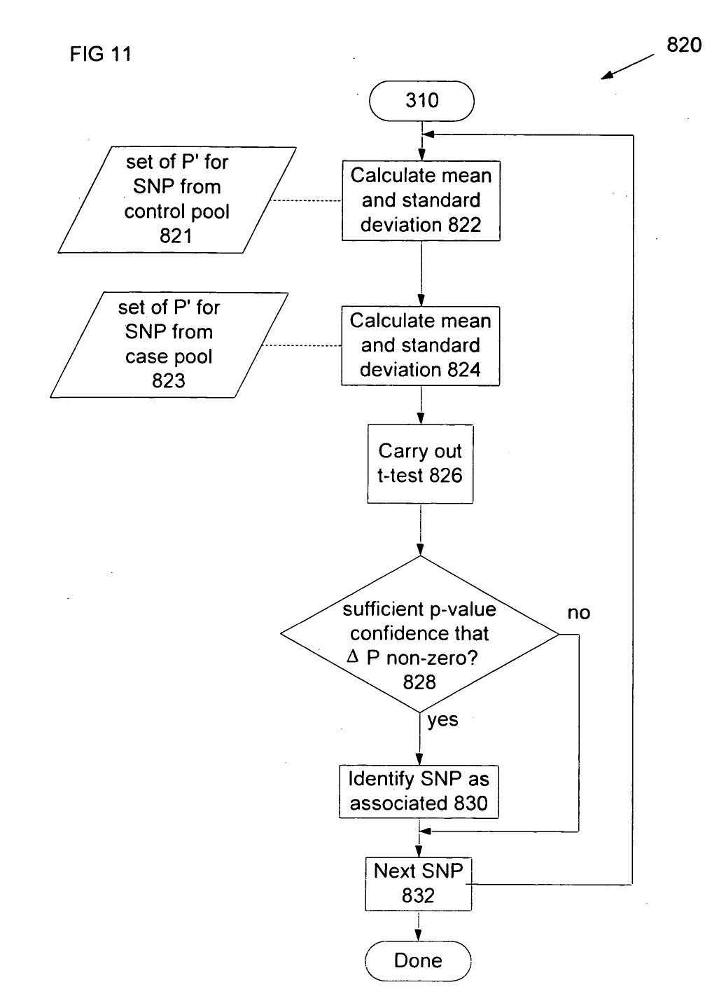

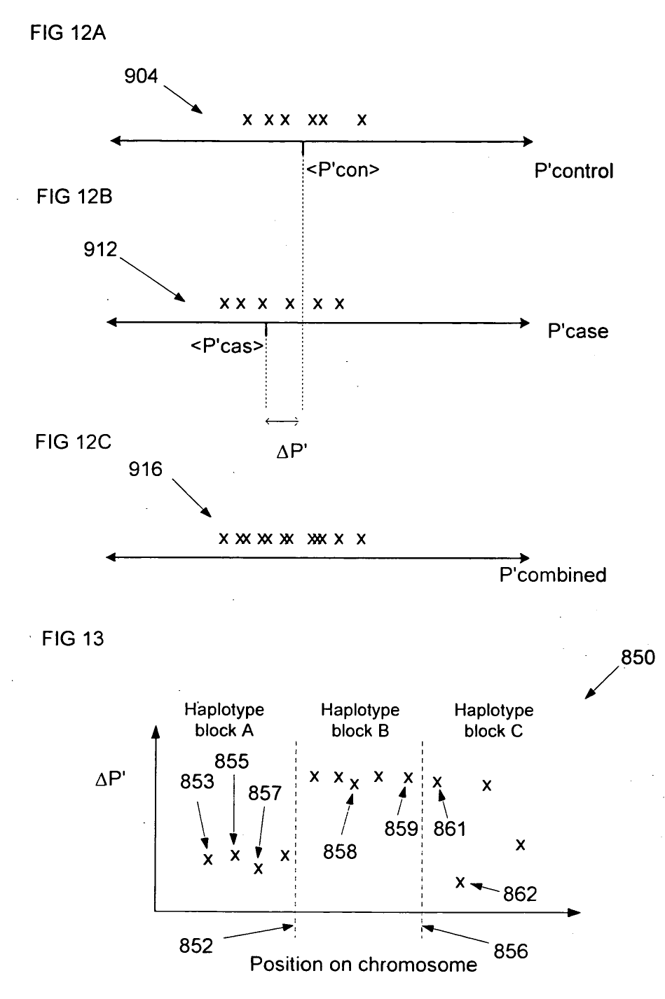

FIG. 11 shows a flow chart illustrating another process 820 which can be used to analyze P′ values to identify associated SNP positions. This analysis process is based on using a t-test to determine the likelihood that the case P′ and control P′ data can be considered to come from different distributions, rather than the same distribution. A target SNP position is selected for evaluation. In the example shown in FIG. 11, for the selected target SNP there is a set of P′s comprising the six P′s for the target SNP from the six experiments for the control pool, i.e. {P′_{1Con}, P′_{2Con} . . . P′_{6Con}} 821. FIG. 12A illustrates the distribution of the values of the set of P′ data items 904 for the control pool 821. The set of P′ values have a distribution which is characterizable by its mean and standard deviation. In the example shown in FIG. 11, there is also a group of P′s comprising the P′ values for each of the six experiments corresponding to the same selected target SNP for the case pool i.e. {P′_{1Cas}, P′_{2Cas} . . . P′_{6Cas}} 823. FIG. 12B illustrates the variation in the values of this group of P′ values 912 for the case pool 823. This group of P′ values has a distribution characterizable by its mean and standard distribution. FIG. 12C shows the variation of a combined group of P′ values 916 comprising the six case and six control P′ values for the selected target SNP.

At step 822 the arithmetic mean <P′_{Con}> and the standard deviation σ_{Con} for the control group P′ values for the selected SNP are calculated. At step 824 the arithmetic mean <P′_{Case}> and the standard deviation σ_{Case} for the case group P′ values for the selected SNP are calculated.

The method then carries out a t-test at step 826 to determine whether it is unlikely that the control group and case group P′ values come from the same distribution. If this is the case, then it can be considered likely that the selected target SNP is associated with the disease as the frequency of occurrence of the reference or alternate allele at the selected target SNP position is different in the case group and the control group. However, if the P′ values appear to come from the same underlying distribution, then it is likely that the selected target SNP position does not distinguish the case and control groups as the frequency of occurrence of the alternate or reference allele at the SNP position can be considered to be statistically the same in the case and control groups.

A null hypothesis approach is used in which it is assumed that the P′ values for the case and control groups come from the same distribution and so ΔP′=0. A two sided t-test is used as ΔP′>0 and ΔP′<0 can both be indicative of an association between the chosen SNP and the phenotype of interest. The t-test returns a t-statistic which is used, together with the number of degrees of freedom (N_{Case}+N_{Con}−2), which is 10 in the event that two groups of 6 P′ data items are used, to obtain a p-value from a standard look up table. The p-value provides a measure of consistency of the data with the null hypothesis. A low p-value indicates that ΔP′=0 is unlikely, therefore the P′ values can be considered likely to come from different distributions.

The statistical significance of the P′_{Case} and P′_{Con} groups of values is assessed using a t-test 826. In particular the t-statistic is computed in one embodiment using:

$$t = \frac{〈 {P}_{Case}^{′} 〉 - 〈 {P}_{Con}^{′} 〉}{σ \sqrt{\frac{1}{{N}_{Case}} + \frac{1}{{N}_{Con}}}}$$

Where σ=\sqrt{[(N_{Case}−1)σ²_{Case}+(N_{Con}−1)σ²_{Con}]/(N_{Case}+N_{Con}−2)}
where N_{Case} and N_{Con} are respectively the number of P′ case and control data items in the respective distributions and σ is the sample standard deviation under the null hypothesis. The t-statistic is then compared with a Student t distribution using the number of degrees of freedom (N_{Case}+N_{Con}−2) to obtain a p-value. The p-value is then assessed at step 828 to determine whether it is appropriate to reject the null hypothesis. A p-value of 0.01 corresponds to a 1% probability of observing as large a difference between cases and controls under the null hypothesis, and a p-value of 0.05 corresponds to a 5% probability. That is, for the latter case, five out of every hundred P′ distributions will be wrongly characterized as being separate P′ distributions rather than the same P′ distribution. In one embodiment a p-value of 0.05 is used in step 828, which provides a 95% confidence level, so that a SNP is identified as an associated SNP in step 830 if the t-test returns a 0.05 or lower p-value.

The process is then repeated for another one of the selected target SNP positions to determine whether that target SNP can be characterized as associated with the disease or not. The process is carried out until all of the target SNP positions have been evaluated and results in a set of data 50 identifying a set of associated SNP positions which have been characterized as being likely to be associated with the case group disease (or other phenotypic trait).

In this way, the large number of target SNPs (typically of order 10⁶) used in the initial first tier association study experiments can be reduced by typically 99% as the target SNPs are segregated into those SNPs for which there is some evidence that ΔP′ is non-zero and those for which this is not the case. A second tier association study can then be carried out 52 for the same case and control groups but with a greatly reduced set of target SNPs which have now been characterized as more likely to be associated with the phenotype of the case group and those characterized target SNPs are taken into account in redesigning the arrays and the experimental protocol for the second tier association study experiments. In some cases, individual genotyping analysis rather than pooled genotyping analysis,is used in the second round.

Different p-values can be used in the first and second tiers of the general method. A lower confidence level may be used in the first tier to provide a relatively coarse filter to identify associated SNPs, and then a higher confidence level can be used to provide a finer filter for the reduced number of SNPs in the second iteration.

Also, in certain alternative embodiments, different confidence levels may be used for the SNP association criterion in step 828 depending on the P′ values. The t-test is a parametric test and assumes that the distributions being considered are normal. However, in practice the P′ distributions may not be normal, although the t-test method is still considered to provide reliable results. It has been found that P′ values close to 0.5 have a closer to normal distribution than those closer to 0 and 1. Therefore different confidence levels may be used depending on the P′ values of the distributions being evaluated. P′ values close to 0.5 may use a more stringent criterion as the t-test may be more applicable to the corresponding P′ distributions than P′ values close to 0 or 1.

iii) Analysis of P′ Data by Using Genetic Information

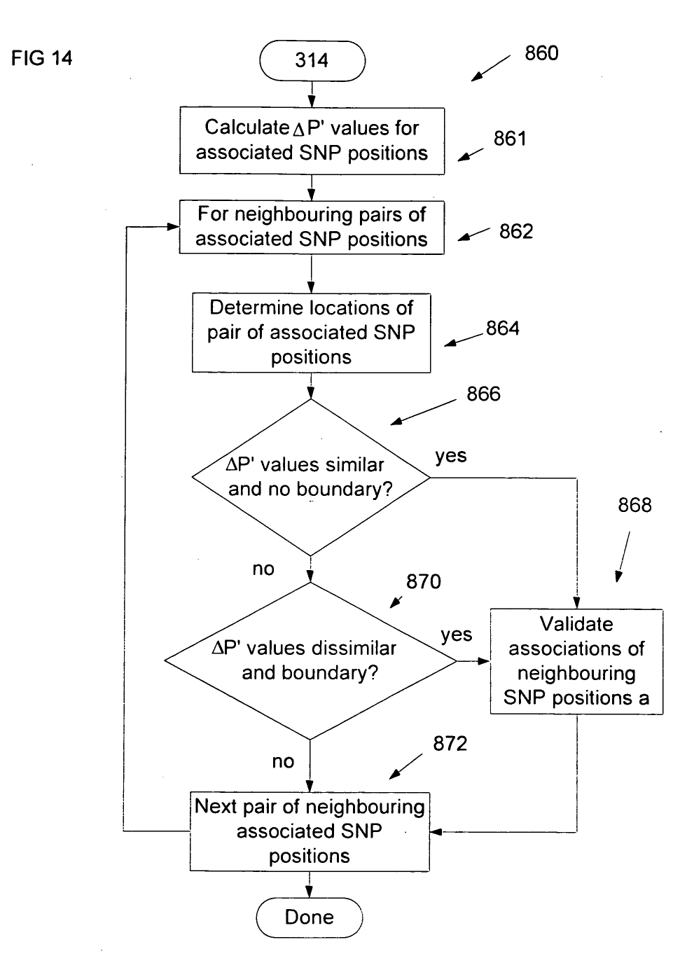

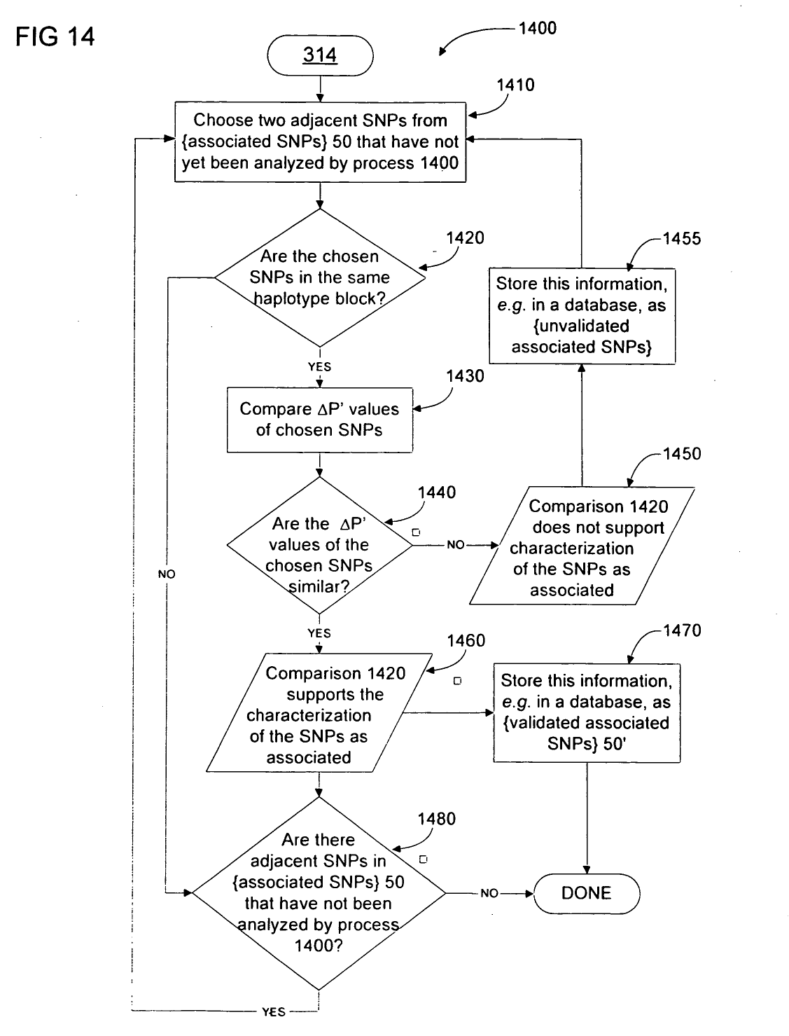

Another class of processes for analyzing the P′ data will now be described with reference to FIGS. 13 and 14. These processes use genetic information to help identify associated SNPs or to validate the associations identified by the analyses described above. FIG. 13 shows a plot 850 of the difference between the P′ values (ΔP′) for the case group and the control group, for 13 target SNPs, against the position of the target SNPs on a chromosome. This method of analyzing P′ data can either be used as a subsequent confirmation of the characterization of a SNP, for example, validation step 314 of the general process 300, or as a separate and independent method of analyzing the SNP P′ data, corresponding to step 310 of process 300. Also the method can use P′ values for the case and control group for a particular target SNP from a single experiment (i.e. ΔP′=P′_{case}−P′_{control}) or can use P′ values averaged over a number of experiments, e.g., as determined at steps 806 and 808 of process 800, to calculate the difference (i.e. ΔP′=<P′_{case}>−<P′_{control}>). It will be appreciated that ΔP′ can also be calculated by subtracting P′_{case} from P′_{control} and so the ΔP′ used is arbitrary as long as the same ΔP′s are used and compared consistently.

As described in U.S. patent application Ser. No. 10/106,097, incorporated herein in its entirety, alleles (variants) making up blocks of polymorphisms (e.g., SNPs) are often correlated or “linked”, resulting in reduced genetic variability and defining a limited number of “SNP-haplotype patterns”, each of which reflects descent from a single, ancient ancestral chromosome (Fullerton, et al., Am. J. Hum. Genet. 67:881 (2000)). As is well known in the art, the term “linkage disequilibrium” or “linked” refers to genetic loci that tend to be transmitted from generation to generation together; e.g., genetic loci that are inherited non-randomly. “SNP haplotype block” means a group of variant or SNP locations that do not appear to recombine independently and that can be grouped together in blocks of variants or SNPs. In other words, a haplotype block is a set of correlated SNPs in a genomic DNA sequence. Typically, haplotype blocks found in genomic DNA sequences extend from a few kilobases up to greater than 100 kilobases. The term “SNP haplotype pattern” refers to the set of genotypes for SNPs in a SNP haplotype block for a single DNA strand. In other words, a haplotype pattern refers to an arrangement of one or more polymorphic nucleotides on a particular chromosome within a haplotype block. The haplotype pattern preserves the information of the phase of the SNPs, i.e., which set of SNPs was inherited from one parent, and which from the other.

This analysis process utilizes the observation that loci that are genetically linked, for example within a haplotype block, tend to be inherited together. Therefore, when one polymorphism is directly involved with the manifestation of a phenotypic trait of interest (e.g., causes a change in a protein directly responsible for the trait), other polymorphisms that are linked to the first will also be “associated” with the phenotypic trait, even if they are not directly involved in the trait. As such, multiple SNPs may be associated with a phenotypic property simply due to the fact that they are linked to a SNP that is directly involved in the manifestation of the phenotypic property. So, the characterization of one SNP as associated may be reinforced by the characterization of another SNP as associated if that other SNP is in the same haplotype block of DNA.

The characterization of one SNP as associated can be further reinforced by comparing the case and control group haplotype patterns that include at least one of the polymorphisms, and haplotype blocks that include at least one haplotype pattern. For example, sets or patterns of polymorphisms that occur at a higher or lower frequency in one population than in another indicate areas in the genome where phenotypic trait-related loci may be located. Optionally, the characterization may be reinforced by comparing the haplotype structures of a region of interest present in case and control groups to identify those polymorphisms or haplotype patterns that associate with a phenotypic trait of interest.

Therefore a first process includes assessing the physical separation of two SNPs with the assumption being that the closer two SNPs are to one another, the more likely it is that they are linked to one another. A ΔP′ value is calculated for each SNP and their proximity is determined. If the ΔP′ for both SNPs is greater than a threshold value, e.g., 0.02, and the separation of the SNPs is less than approximately 100 kilobases, then the two SNPs may both be characterized as associated. In other embodiments, the SNPs can be considered to be sufficiently proximate if their separation is less than approximately 50 kb, and preferably approximately 10 kb or less. Without wishing to be bound by theory, SNPs that are close to one another are more likely to be part of the same block of DNA than SNPs that are far apart, and that block of DNA may be involved in the disease mechanism.

A second similar process includes varying the proximity criterion using knowledge of the length of DNA blocks that are inherited. For example, a haplotype block map can be used to provide information as to the length of the block in which a first SNP with a finite ΔP′ is located. Examples of how to construct a haplotype map are described in detail in U.S. patent application Ser. Nos. 10/284,444, 10/166,341, and 60/323,059, all of which are incorporated by reference herein. The proximity criterion can then be adjusted based on the length of the block containing the first SNP. For example, if the haplotype block is approximately 20 kb long and the first SNP is located in the middle of the block, then only SNPs separated by less than approximately 10 kb from the first SNP, and therefore on the same block need have their ΔP′s evaluated, to see if the SNPs can be characterized as associated. In other words, if a first SNP is found to be associated with a phenotypic trait of interest, than one would expect that other SNPs in the same haplotype block as the first SNP are likely to also be associated with the same phenotypic trait of interest. If this is found not to be the case, then the first SNP's association with the phenotypic trait may be called into question.

The location of points of high recombination (owing to the cross-over of chromosomes during meiosis) can be used to define the size of blocks of DNA, as in general a recombination “hotspot” generally is not found within a haplotype block since the SNPs in such blocks tend to be inherited together. As such, typically recombination hotspots only occur between haplotype blocks. The position of such recombination hot spots can also be used in characterizing SNPs. For example if two SNPs are sufficiently close together to meet the proximity criterion and their ΔP′s are sufficiently similar, if they are on separate sides of a recombination hot spot then they are unlikely to be inherited together and so they would not likely be in the same haplotype block. So, if one SNP was shown to be associated with a phenotypic trait of interest, the putative association of the other SNP could not be assumed and would have to be determined independently. Further, the association of one SNP could not be used to validate the association of the other.

Another process uses SNP clustering within haplotype blocks to characterize a SNP position. Since the borders of haplotype blocks may be determined in various ways, SNPs that are clustered within a haplotype block are more likely to remain in that haplotype block even after a reanalysis of the haplotype structure that changes one or both borders of the haplotype block than are SNPs that are far apart in a block. That is, clustered SNPs are more likely to be linked to one another than are SNPs that are widely dispersed. This process therefore determines the ΔP′ values for a number of SNPs that occur in clusters in a haplotype block, and if the ΔP′ values are sufficiently similar and are above a given threshold then the selected SNPs can be characterized as associated.

Haplotype blocks are inherited and may be associated with the genetic basis for a disease. Irrespective, it has been observed that ΔP′s of SNPs that are associated with the disease tend to cluster around a similar value depending on the proximity of the SNPs on the chromosome. Haplotype blocks are typically on the order of 10s of kbs (kilobases) long and can include several to over 100 SNPs. Hence by assessing the similarity and proximity of ΔP′ values, and using haplotype map information, SNP locations can be characterized as associated or not, or their characterization reinforced by this secondary genetic information.

For example, the x's in FIG. 13 indicate the ΔP′ values for thirteen target SNP positions that have previously been characterized as being associated by, for example, method 800. As shown in FIG. 13, the SNPs in haplotype block A have similar ΔP′ values to one another, and the SNPs in haplotype block B have similar ΔP′ values to one another. If position 852 corresponds to a haplotype block boundary as identified from a haplotype block map, then these observations reinforce the identification of the SNP positions on either side of the boundary position as being associated with the disease. The fact that those in haplotype block A have significantly different ΔP′ values than those in haplotype block B does not invalidate the association with the disease since haplotype block boundary 852 separates the SNPs in A from those in B demonstrating that the SNPs in A are not expected to be linked to those in B. If position 852 does not correspond to a haplotype block boundary, then the aforementioned observations do not reinforce belief in the association of the haplotype block A and haplotype block B SNPs with the phenotype of interest, as otherwise the ΔP′ values on either side of position 852 would be expected to be more similar. Similarly, if position 856 did not correspond to a boundary between haplotype blocks, then that observation might not reinforce a belief that the SNP positions in haplotype block B were associated SNP positions since SNPs in haplotype block C have significantly different ΔP′ values than those in haplotype block B.

Now consider that position 856 is a haplotype block boundary that separates haplotype block B from haplotype block C. The ΔP′ value 861 in haplotype block C may have initially been considered to indicate an associated SNP position. However, the difference in its ΔP′ value as compared to the ΔP′ values for the other SNP positions in haplotype block C may be considered sufficient to indicate that it was falsely characterized as an associated SNP position. The fact that it is close in value to the ΔP′ value 859 is not sufficient to validate its association since haplotype block boundary 856 falls between the two positions.

Hence, the similarity of ΔP′ values for associated SNP positions, the proximity of the associated SNP positions, and/or haplotype map data can be used to provide a secondary indication of the validity of the characterization of a SNP position as being an associated SNP position.

FIG. 14 illustrates the steps of an exemplary process 1400 by which associated SNPs can be validated using the above enumerated principles. In light of the discussion above, it will be appreciated that other methods can be used to validate the characterization of associated SNP positions and that this embodiment is by way of an example.

For example, another means of validating an associated SNP position includes identifying a location of the nucleic acid segment in the human genome. The SNP position may occur within the coding or noncoding regions of a gene. It may be in a region that regulates the expression of a gene, such as a promoter or enhancer region. It may be in an exon or intron of a gene. If an associated SNP position is located in a coding or regulatory region of a gene, then the gene may be deemed to be associated with the phenotypic characteristic of interest. In such a case, the step of validating may further include cloning and expressing the associated gene to produce and/or characterize a protein product, or regulating expression of the associated gene in cells in vitro or in vivo and detecting changes of cells in response to the regulation. In certain embodiments, once an associated gene has been identified, the step of validating may further include screening a library of pharmaceutical candidates against the associated gene or gene product, and selecting the pharmaceutical candidates that modulate expression of the associated gene or activity of the associated gene product. These pharmaceutical candidates may also be screened to determine whether or not they have an effect, either direct or indirect, on the phenotype of interest. This screening may be performed in numerous ways well known to those of skill in the art including, but not limited to, administering the pharmaceutical candidate and monitoring the phenotype of interest for any changes in response to the pharmaceutical candidate. Other methods of use of associated SNP positions, associated genes, and pharmaceutical candidates -are described in detail in U.S. patent application Ser. No. 10/106,097, filed Mar. 26, 2002, entitled “Methods for Genomic Analysis”, which is incorporated herein by reference in its entirety for all purposes.

For each SNP position that has been characterized as an associated SNP position, a ΔP′ value was calculated, for example in step 812 of FIG. 10, by subtracting the P′ value for the SNP position for the control group from the P′ value for the same SNP position for the case group for an experiment. It will be appreciated that whether to subtract the case group P′s from control group P′s to obtain ΔP′, or vice versa, is merely a matter of convention and either can be used to obtain a ΔP′, provided that ΔP′ is used consistently. In an alternative embodiment, average P′ values are used to calculate ΔP′. The average P′s can be obtained by calculating the mean P′ value for the SNP position from a set of replicated experiments. Each SNP position has a particular position on the chromosome and a ΔP′ value, as illustrated in FIG. 13.

As explained above, the similarity of ΔP′ values for associated SNP positions and their position with respect to haplotype block boundaries can be used to validate the characterization of the SNP position as an associated SNP position. A first two associated SNPs that are adjacent to one another are identified 1410 for evaluation, for example from the set of {associated SNPs} 50 shown in FIGS. 3 and 5. The positions of the adjacent SNPs are determined by referring to stored information on the chromosomal locations of the associated SNP positions and/or the haplotype structure of the chromosome, and a determination is made as to whether the SNPs lie in the same haplotype block 1420. In other words, it is determined, by looking up haplotype block map data, whether a haplotype block boundary or other DNA recombination boundary is believed to occur between the neighboring SNP positions. The ΔP′ values for adjacent SNPs that are in the same haplotype block (i.e., not separated by a haplotype block boundary or other DNA recombination boundary) are compared 1430 to see whether they differ by more than a threshold value 1440. If they do, then the ΔP′ values are considered not to be sufficiently similar and this data would not support the validation of the characterization of the SNPs as associated 1450; this information would be stored 1455, for example, in a database as {unvalidated associated SNPs}. One example of this would be SNP positions 861 and 862 shown in FIG. 13. If the ΔP′ values for the adjacent SNPs are sufficiently similar, then this data would support the validation of the characterization of the SNPs as associated 1460; this information would be stored 1470, for example, in a database as {validated associated SNPs} 50′ (see also FIG. 5). One example of this would be SNP positions 853 and 855 shown in FIG. 13.

If the ΔP′ values are dissimilar and there is no boundary (e.g. SNP positions 861 and 862 in FIG. 13), then this is an indication that one or both of the SNP positions may have been incorrectly characterized as an associated SNP position, as this contradicts the general observation that ΔP′ is approximately the same for SNPs on the same haplotype block. Therefore the association is not validated. However, this merely raises a query as to the validity of the characterization of the SNP position as associated and does not necessarily negate the characterization of the SNP position. The lack of validation may be used as a flag to indicate that the SNP positions need further investigation or that less weight should be placed on the characterization of either or both of the SNP positions as being associated SNP positions.

More than one block may be associated with a phenotype (for example, a single gene may contain more than one block or may overlap a block boundary, or associated blocks may be dispersed across a chromosome or a genome when multiple genes are involved). Therefore, the use of haplotype data can provide greater reliability by way of validating SNPs that have been characterized as associated based on their respective ΔP′s.

Further, the existence of a boundary does not necessarily require a difference in ΔP′ values between adjacent haplotype blocks and so a determination that the ΔP′ values are similar and there is a boundary is not inconsistent with the validity of a characterization of the SNPs as associated.

At this stage the method has therefore validated, or not, the characterization of the first two adjacent SNP positions as associated. In the example, the ΔP′ values for 853 and 855 are similar and there is no boundary and this is consistent with both 853 and 855 being associated SNPs. The method then determines whether there are adjacent SNPs in {associated SNPs} 50 that have not been analyzed by process 1400. If there are, then another adjacent pair of associated SNP positions are analyzed, for example 855 and 857 in FIG. 13. If there are no more adjacent SNPs in {associated SNPs} 50 that have not been analyzed by process 1400, then process 1400 is complete.

Hence, as can be seen, although the fourth and fifth associated SNP positions in FIG. 13 have different ΔP′ values, their characterization as associated may be valid as there is a boundary 852 between them. SNP position 862 may not be validated as its ΔP′ value may be considered sufficiently dissimilar to its neighbors and there is no boundary between them. Further, the difference between the ΔP′ values for the ninth 859 and tenth 861 SNP positions may be considered sufficiently similar, within the variations of the experimental data or otherwise, that even though there is a boundary 856 between them, this does not militate against the validity of their characterization as associated.

Various modifications and additions to this general methodology of using similarity and position data to validate the characterization of SNP positions as associated may be employed. For example, a ΔP′ for a one of the SNP positions on a haplotype block may be compared with an average ΔP′ for the rest of the SNP positions on that haplotype block in order to determine similarity, and a measure of the distribution of the ΔP′ values for the rest of the SNP positions, e.g. the standard deviation, may be used as, or to help determine, a threshold value.

Further, the distance between neighboring SNP positions may be used as a supplementary validation mechanism, instead of the ΔP′ similarity and boundary validation mechanisms described above.

Haplotype map information can be used to refine allele frequency (P or P′) and/or allele frequency difference (ΔP or ΔP′) data to reduce the rate of false positive associations. In particular, haplotype map information can be used to refine estimates of SNP allele frequency differences between a pooled sample of a case group and that of a control group. The method exploits the fact that within a haplotype block most of the variation in SNP allele frequencies can be accounted for by variation in frequencies of a relatively small set of common haplotype patterns. And, within a block, the sum of changes in these pattern frequencies should be approximately zero, to the extent that those patterns in the haplotype map accurately represent the total genetic diversity of that interval.

In one embodiment of the method, the haplotype map is constructed so that common haplotype patterns account for at least 80% of the genetic variation in a set of more than 20 haploid genomes selected from a globally diverse panel of cell lines. Examples of, how to construct a haplotype map (e.g., Chromosome 21 haplotype map) are described in detail in U.S. patent application Ser. Nos. 10/284,444, 10/166,341, and 60/323,059, all entitled “Human Genomic Polymorphisms”, all of which are incorporated by reference herein.

Optionally, the haplotype map may be constructed to account for a larger or smaller fraction of observed genetic variation; and the map could also be constructed using individuals from a specific population. For example, the map may be constructed from individuals of a particular ancestral heritage or individuals who exhibit a particular phenotype of interest. The specific method of construction of the map does not limit the application of this invention, but it is required that the map assigns SNPs to discrete haplotype blocks, and identifies a limited set of common haplotype patterns within each block.

In a particular embodiment, given ΔP′s for a haplotype block linear regression is used to solve for underlying haplotype pattern frequency differences using the following equation.

$$Δ {P}_{i} \approx {\sum }_{j = 1}^{N} {m}_{ij} Δ {f}_{j}$$

where ΔPᵢ is the difference between the estimated reference allele frequency for SNP i within a haplotype block in a case group and the corresponding frequency in a control group; Δfⱼ is the (unknown) frequency difference for a common haplotype pattern; j∈1 . . . N, where N is the total number of different patterns for the haplotype block; and mᵢⱼ is a coefficient that takes a value of +0.5 if the allele at position i in pattern j matches the reference allele, and −0.5 if it matches the alternate allele for that SNP. The reason for the 0.5 factor is that the frequency difference for an allele would otherwise be double counted when differences for the complete set of patterns are evaluated.

This linear regression model also requires that the sum of pattern frequency differences add up to 0:

$${\sum }_{j = 1}^{N} Δ {f}_{j} = 0$$

This constraint can be folded into the above linear regression equation by substituting:

$$Δ {f}_{N} = - {\sum }_{j = 1}^{N - 1} Δ {f}_{j}$$

to obtain:

$$Δ {P}_{i} \approx {\sum }_{j = 1}^{N - 1} ( {m}_{ij} - {m}_{iN} ) Δ {f}_{j}$$

$$or$$

$$Δ {P}_{i} \approx {\sum }_{j = 1}^{N - 1} {r}_{ij} Δ {f}_{j}$$

where
rᵢⱼ≡mᵢⱼ−m_{iN}

In the linear regression model, the ΔPᵢ are the dependent variables, the \overline{r}ᵢⱼ are the independent variables, and the Δfⱼ are the regression coefficients to be fitted. Either a standard statistical package or a dedicated computer program can be used to perform the linear regression. In the following example, the analysis was done using the R analysis package (http://www.r-project.org). Standard measures (R², and the p-value for an F test) can be used to judge the quality of the fit of the SNP data to the haplotype pattern information. It is noted that deviations from a perfect fit may arise from experimental errors, and/or inaccuracies in the haplotype map.

As an exemplary illustration, the linear regression model described above was applied to refine allele frequency differences between a case and control group at 10 SNP position in a haplotype block. One haplotype block consisting of 10 SNPs was selected based on the haplotype map for Chromosome 21 (as described in U.S. patent application Ser. Nos. 10/284,444, 10/166,341, and 60/323,059, all of which are incorporated by reference herein). The 10 SNPs were identified here by their chromosomal positions in the me assembly build 30 and listed in Table 1 as follows.

TABLE 1

| | | | |
|---|---|---|---|
| SNP | Position | ref | alt |

| | | | |
|---|---|---|---|
| 1 | 26534351 | G | A |
| 2 | 26534833 | T | C |
| 3 | 26535649 | G | T |
| 4 | 26536570 | G | A |
| 5 | 26537228 | A | C |
| 6 | 26537465 | C | T |
| 7 | 26537631 | G | A |
| 8 | 26537674 | A | G |
| 9 | 26537753 | C | A |
| 10 | 26538313 | T | G |

The following four common patterns for this haplotype block had been previously identified described in U.S. patent application Ser. Nos. 10/284,444, 10/166,341, and 60/323,059) and listed here in Table 2:

TABLE 2

| pattern | haplotype |
|---|---|
| 1 | ACTACTAGAT |
| 2 | GTGGACGACT |
| 3 | GCGGACGACT |
| 4 | ACTACTAGCG |

The m matrix, indicating whether a position in a pattern matches the reference or alternate allele, is given by Table 3 shown below:

TABLE 3

| | 1 | 2 | 3 | 4 |
|---|---|---|---|---|
| 1 | −0.5 | +0.5 | +0.5 | −0.5 |
| 2 | −0.5 | +0.5 | −0.5 | −0.5 |
| 3 | −0.5 | +0.5 | +0.5 | −0.5 |
| 4 | −0.5 | +0.5 | +0.5 | −0.5 |
| 5 | −0.5 | +0.5 | +0.5 | −0.5 |
| 6 | −0.5 | +0.5 | +0.5 | −0.5 |
| 7 | −0.5 | +0.5 | +0.5 | −0.5 |
| 8 | −0.5 | +0.5 | +0.5 | −0.5 |
| 9 | −0.5 | +0.5 | +0.5 | +0.5 |
| 10 | +0.5 | +0.5 | +0.5 | −0.5 |

In Table 3, the four numbered columns represent the four common haplotype patterns and the ten numbered rows represent the ten SNPs in the haplotype block. As shown in the m matrix in Table 3, pattern 2 matches the reference allele at every SNP as column 2 all contains +0.5 values.

The r matrix was obtained by subtracting column 4 from the other three columns and is giving by Table 4:

TABLE 4

| | 1 | 2 | 3 |
|---|---|---|---|
| 1 | 0 | +1 | +1 |
| 2 | 0 | +1 | 0 |
| 3 | 0 | +1 | +1 |
| 4 | 0 | +1 | +1 |
| 5 | 0 | +1 | +1 |
| 6 | 0 | +1 | +1 |
| 7 | 0 | +1 | +1 |
| 8 | 0 | +1 | +1 |
| 9 | −1 | 0 | 0 |
| 10 | 1 | 1 | 1 |

In an association study using methods of the present invention, estimates of pooled allele frequency differences (ΔPᵢ) were measured, e.g., by using hybridization to oligonucleotide arrays, and listed in Table 5 as follows:

TABLE 5

| SNP | ΔPᵢ |
|---|---|
| 1 | 0.05477 |
| 2 | 0.03101 |
| 3 | 0.03156 |
| 4 | 0.04154 |
| 5 | 0.05670 |
| 6 | 0.02987 |
| 7 | 0.01725 |
| 8 | 0.02886 |
| 9 | −0.00504 |
| 10 | 0.05623 |

The linear regression model was applied to the values in Table 5 against the r matrix in Table 4 and produced estimates for the pattern frequency differences as listed in Table 6. It is noted that the estimate for pattern 4 in Table 6 was obtained using the rule that the four differences must sum to zero.

TABLE 6

| pattern | Δfⱼ |
|---|---|
| 1 | 0.01156 |
| 2 | 0.03101 |
| 3 | 0.00714 |
| 4 | −0.04972 |

In this instance, the quality of the fit to the observed SNP frequency differences is fairly high. The regression yielded an R² of 0.91, meaning that 91% of the variance in the original ΔPᵢ data was accounted for by the three independent Δfⱼ values. An F-test for the fit gave a p-value <0.0005 indicating the fit is quite unlikely to arise by chance. Using the calculated Δfⱼ values in Table 6 and the r matrix in Table 4, fitted values for the individual SNP frequency differences were generated and listed in Table 7.

TABLE 7

| SNP | ΔPᵢ | fitted |
|---|---|---|
| 1 | 0.05477 | 0.03815 |
| 2 | 0.03101 | 0.03101 |
| 3 | 0.03156 | 0.03815 |
| 4 | 0.04154 | 0.03815 |
| 5 | 0.05670 | 0.03815 |
| 6 | 0.02987 | 0.03815 |
| 7 | 0.01725 | 0.03815 |
| 8 | 0.02886 | 0.03815 |
| 9 | −0.00504 | −0.01156 |
| 10 | 0.05623 | 0.04972 |

These fitted allele frequency differences may be used to inform the selection of SNP's as candidates for further analysis, which might include individual genotyping in the same or different cohort to validate putative associations. In instances where the quality of the fit to the haplotype map is good, the fitted allele frequency differences should have lower variance than the raw data for individual SNPs, because they incorporate information about the expected correlations between SNPs.

According to the linear regression model described above, to permit estimation of frequency differences for N patterns in a haplotype block, data for at least N−1 SNPs should be available. The haplotype map may be constructed such that a block with N common haplotype patterns must contain at least N−1 SNPs, so given complete data, this requirement is always satisfied. Where exactly N−1 SNPs are present, there is no additional correlation information in the haplotype map. Preferably, data for more than N−1 SNPs is available per haplotype block. Inclusion of rare haplotype patterns in the analysis would make the relative numbers of SNPs and patterns less favorable for the method.

In one embodiment, the linear regression analysis is performed on allele frequency differences. One of the advantages associated with this method is that it is invariant to some systematic errors in the pooled allele frequency estimates. The method is valid as long as the pooled measurement is strongly correlated with actual allele frequency. If the measurement is systematically scaled or offset with respect to actual frequencies, any offsets will cancel out in the calculation of frequency differences, and the resulting fitted values will be in the same scale as the measurements. As in the example described above, allele frequency estimates were made based on relative intensities from hybridization to oligonucleotides. These intensity ratios are somewhat inaccurate measures of absolute allele frequencies, but are well suited to detecting small frequency differences between pools. The linear regression analysis of the present invention can be performed on these estimated allele frequency differences to render the data closer to the true values.

Optionally, the linear regression analysis of the present invention may be used to estimate the absolute frequencies (i.e., P or P′ values) of the haplotype patterns in a single pool, e.g., a sample from an individual or collected from a group of individuals. The P values may be measured by using the oligonucleotide hybridization assay described above or other genotyping methods known in the art. Then the linear regression analysis of the present invention is used to refine the data by using the following relationship:

$${P}_{i} \approx {\sum }_{j = 1}^{N} {m}_{ij} {f}_{j}$$

where mᵢⱼ takes a value of +1 if the allele at position i in pattern j matches the reference allele, and 0 if it matches the alternate allele for that SNP. The frequencies are also constrained by:

$${\sum }_{j = 1}^{N} {f}_{j} = 1$$

This constraint can be used to reduce the number of free parameters in the regression by one. It also may be useful to constrain the regression such that 0≦fⱼ≦1 to avoid impossible solutions.

These methods use allelic information in haplotype patterns from the separately derived haplotype map, but do not use information about SNP allele frequencies or haplotype pattern frequencies in the data used to derive the map. The linear regression methods for refining P and ΔP values should be generally applicable to analysis of data obtained from various population or subpopulations. In one situation, the haplotype map may be derived from one population of individuals whereas the P and/or ΔP values may be derived from measurement of relative allele frequencies of another, different population. In another situation, although less common, the haplotype map and P and/or ΔP values may be derived from the same population of individuals. While there is much evidence that SNP allele frequencies (and by extension, haplotype pattern frequencies) vary widely between different human subpopulations, it is believed that the haplotype boundaries and common patterns observed in a globally diverse population should largely be a superset of the boundaries and patterns observed in more narrowly derived subpopulations. Consequently, these methods may tend to conservatively underestimate the amount of actual SNP redundancy.

The methods described above can be used to further characterize the associated SNPs, thereby identifying SNP positions which may merit further investigations or to provide a weighting which may be given to the data in subsequent analysis.

iv) Analysis Based on Distribution of ΔP′ Values

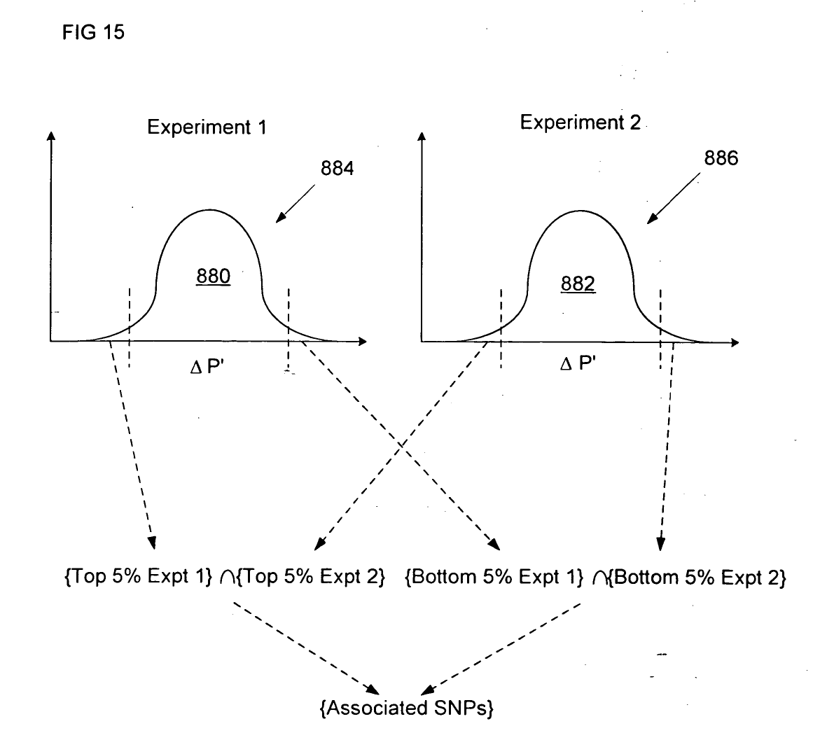

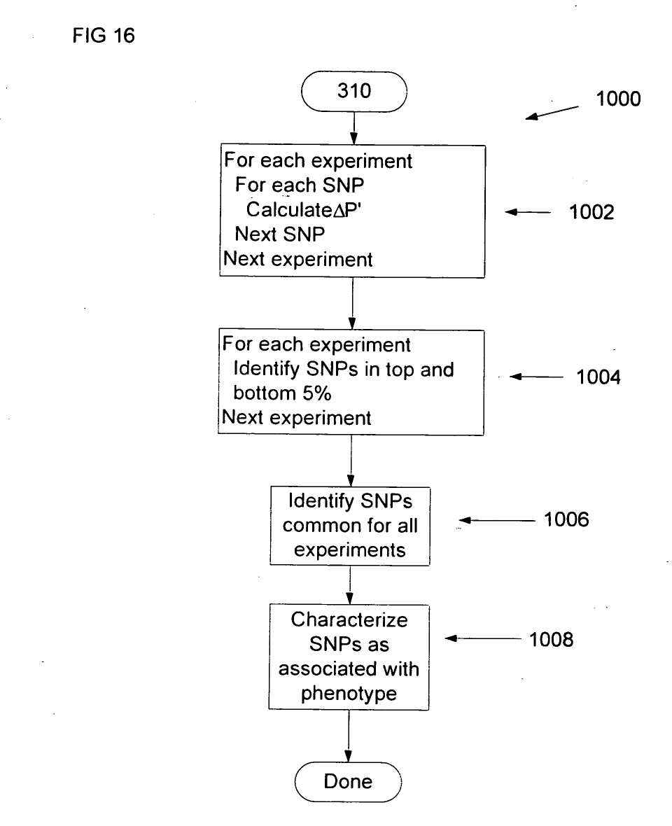

A further class of processes for analyzing the P′ values to characterize SNP positions as associated or not will now be described with reference to FIGS. 15 and 16. These methods correspond to alternative processes for analysis step 310 of the general method illustrated in FIG. 5. These methods select SNPs having ΔP′ values that are consistently found in the extreme tails of the ΔP′ distribution in repeated experiments.

FIG. 15 shows a graph 884 illustrating the distribution of ΔP′ values 880 for all the SNP positions to be characterized for a first experiment and a graph 886 illustrating the distribution of ΔP′ values 882 for all the SNP positions to be characterized for a second experiment replicating the first experiment. Therefore there are two experimentally determined ΔP′ values for each SNP position, one from each distribution. The extreme ΔP′ values and corresponding SNPs are evaluated for each experiment. Generally, ΔP′ will be centered somewhere near zero and one end of the ΔP′ scale corresponds to positive values (top) and the other end of the scale (bottom) corresponds to negative values.

In a first analysis process, the distribution of ΔP′s for all the SNPs for a single experiment is determined. The associated SNPs are then identified as corresponding to those SNPs that fall in the top 0.5% and the bottom 0.5% of the distribution (or alternatively the top 1% if |ΔP′| is used). In general associated SNPs are rare, therefore taking extreme ΔP′ values as being indicative of an associated SNP can be used to remove the large majority of non associated SNPs from the large number of SNPs being evaluated. Other cut off criteria can be used. For example the top and bottom 1-10% of ΔP′ values can be used. The SNP positions having ΔP′ values falling within the extreme parts of the distribution are then identified as candidate associated SNPs.

Another analysis procedure uses distributions of ΔP′ values for all SNPs from separate replicated experiments, as illustrated in FIG. 15 and shown by the flowchart of FIG. 16. The top and bottom 5% of ΔP′ values for the first experiment and the top and bottom 5% of ΔP′ values for the second experiment are determined and the corresponding SNP positions identified. For an experiment, the average value of ΔP′ over all the SNPs, <ΔP′>, tends to be non-zero and the distribution of ΔP′ values typically has a standard deviation of approximately 1%.

If none of the SNP positions are associated, then the set of ΔP′ values will not be systematically biased. Therefore it is possible to estimate the number of SNPs that will fall within the top 5% or bottom 5% for repeated experiments, in the absence of any associations. Deviations away from that estimated number can therefore be taken as an indication of the existence of associations and the SNP positions whose ΔP′ values consistently fall within the extreme parts of the distribution for repeated experiments can be considered likely to be associated SNP positions and characterized as such.

An embodiment of a data processing method embodying the principles enumerated above will now be described in further detail with reference to the flow chart in FIG. 16. In this example, an experiment on the same control and case group has been replicated and there are two sets of experimentally determined P′ values covering each of the 1.7 million candidate SNPs, from which non-conforming data has been rejected. Initially, ΔP′ values for each of the conforming 1.7 million SNP positions are calculated at step 1002 for the first experiment and for the second experiment. This produces a distribution of ΔP′ values for the first experiment and the second experiment, as illustrated in FIGS. 15A and 15B respectively. For the first experiment, the SNP positions corresponding to the ΔP′ values falling in the highest 5% and lowest 5% of the distribution of ΔP′ values are identified at step 1004. This identifies a set of approximately 85,000 SNP positions corresponding to the top 5% {Top 5% Expt1} and a further set of approximately 85,000 SNP positions {Bottom 5% Expt 1} corresponding to the bottom 5%, and different to the set of ‘top 5%’ SNP positions. The process is repeated for the second experiment, identifying a set of top 5% SNP positions Top 5% Expt2} and a set of bottom 5% SNP positions {Bottom 5% Expt2} for the second experiment.

The method then identifies at step 1006 any SNP positions whose ΔP′ values occur in the top 5% in both experiments (replicates) {Top 5% Expt1}∩{Top 5% Expt2} and SNP positions whose ΔP′ values occur in the bottom 5% in both experiments {Bottom 5% Expt1}∩{Bottom 5% Expt2}. On the assumption of no associated SNP positions, the number of SNP positions whose ΔP′ values will fall within the top 5% in two experiments by chance would be approximately 5% of 5% of 1.7 million, which is approximately 4,250, and similarly for the bottom 5%. Therefore, if there are substantially more than 4,250 SNP positions common to the top 5% in both experiments, the some of them are likely associated SNP positions, and similarly for the bottom 5%. Although some of the SNP positions common to both experiments will by chance be non-associated, a significant number of the members of the set of SNPs common to the top 5% of both experiments will be associated SNP positions. As will be appreciated, this has reduced the initial set of 1.7 million candidate SNP positions by approximately three orders of magnitude and the reduced set of SNP positions can be characterized 1008 as likely being associated SNPs which can be investigated in greater detail.

If more than two sets of experimental results are available, then the number of SNP positions having ΔP′ values in the top 5% which are common to all of the experiments by chance will be reduced by 5% for each experiment. Therefore, for example, if six experiments have been carried out, the number of SNP positions that by chance have ΔP′ values falling within the top 5% in all six experiments would be approximately (0.05)⁶×1.7 million which is on the order of about 0.0266. Therefore any SNP positions common to all six experiments can be characterized 1008 as likely to be SNP positions associated with the phenotype of the case group under investigation. Therefore the intersection of the sets of SNP positions with ΔP′ values falling within the top 5% for different experiments will provide a subset of SNP positions likely to be associated with the phenotype. The certainty of the characterization of the SNP positions as associated will increase with the number of experiments. Further, for reduced initial candidate SNP sets, fewer experiments may be needed in order to provide a suitable level of statistical certainty. Furthermore, a reduced proportion of the distribution of ΔP′ values can be used to reduce the number of repeated experiments required in order to more definitively identify associated SNPs (e.g., a top 1% or 2%). However, that runs the risk of excluding SNP positions which are associated and so an increased number of repeated experiments may be preferred.

The set of SNP positions characterized as associated {associated SNPs} 50 (FIG. 3) can then be used for second tier association experiments, as described above, and/or validation of the set of associated SNP positions 50 can be carried with reference to haplotype block data according to the method described above.

v) Analysis Based on P′ from Forward/Reverse Probe Tiling

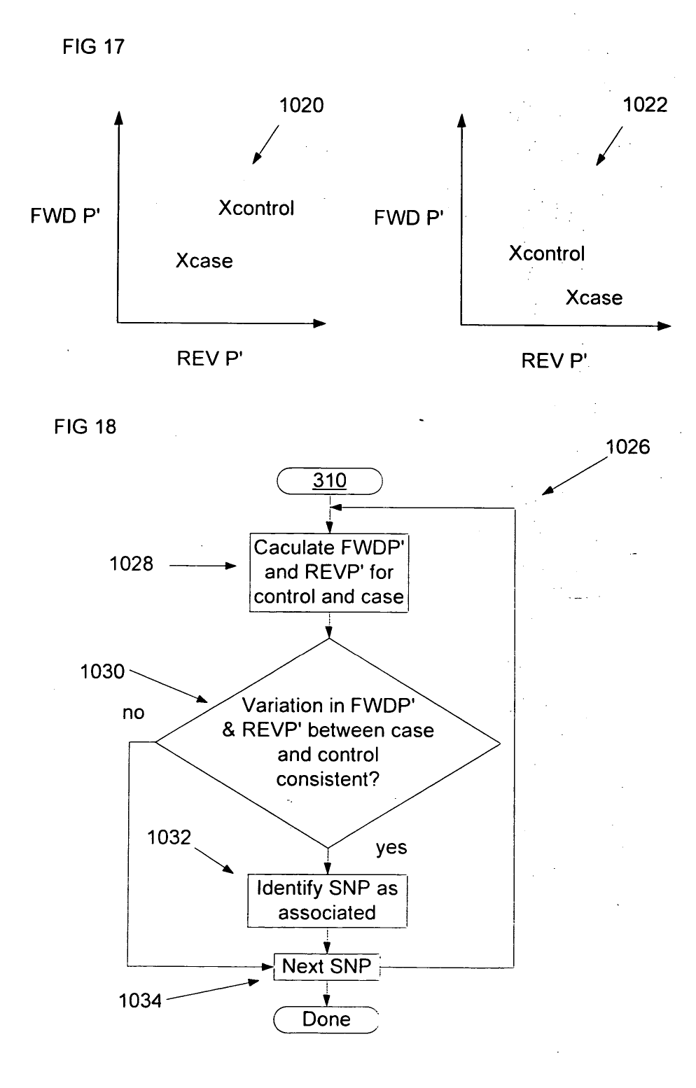

Another process 1026 for analyzing P′ data to identify associated SNPs, corresponding to method step 310, will now be described with reference to FIGS. 17 and 18. This process 1026 uses P′ data and is based on the availability of two independent, although slightly different, measures of P′ being available for a single experiment from the forward sequence and reverse sequence probe tilings for each SNP. Rather than calculating P′ averaged over all ten probes in process 700, an average P′ is calculated using the intensity measurements for the five forward probes and an average P′ is calculated using the intensity measurements for the five reverse probes. So an average forward probes P′, FWDP′, and an average probes P′, REVP′, is determined for each SNP position, for the case group and the control group 1028. The process is based on an expectation that if an association is genuine, then the FWDP′ and the REVP′ will vary in the same way between the control and case groups. If the FWDP′ and the REVP′ changed in opposite ways between the control and case groups, then this is an indication that the data is unreliable and therefore it cannot be used to determine whether or not the SNP is associated.

FIG. 17 shows a graphical representation of a two dimensional space illustrating the variation in FWDP′ and REVP′ for the control and case groups for an instance 1020 where the variation is consistent with the SNP being associated and an instance 1022 where the variation is inconsistent with the SNP being associated. In the consistent instance, FWDP′ is greater in the control group than the case group and REVP′ is also greater in the control group than the case group. The alternate consistent instance is the FWDP′ being lower in the control group than the case group and the REVP′ also being lower in the control group than the case group. Therefore a consistent variation can be considered to be both REVP′ and FWDP′ being either higher or lower in the case group as compared to the control group. A line passing through the corresponding control group datum Xcontrol and the case group datum Xcase would have a positive gradient, which is indicative of a consistent scenario.

In contrast, in the inconsistent instance, FWDP′ is higher in the control group than in the case group, but REVP′ is lower in the control group than in the case group. The alternate inconsistent instance is FWDP′ being lower in the control group than in the case group and the REVP′ being higher in the control group than in the case group. Therefore an inconsistent variation can be considered to be REVP′ and FWDP′ changing with opposite signs between the case and control groups. A line passing through the corresponding control group datum Xcontrol and the case group datum Xcase would have a negative gradient, which is indicative of an inconsistent scenario.

The process 1026 therefore determines at step 1030 whether the variation in FWDP′ and REVP′ between the case and control groups is consistent, for example by determining the sign of the gradient in the virtual, two-dimensional FWDP′ and REVP′ space. If it is determined that the variation is consistent, then the SNP being evaluated is identified as likely being an associated SNP 1032 and a next SNP is selected for evaluation at step 1034. Otherwise, the SNP is not identified as likely being associated and a next SNP is selected for evaluation 1034.

vi) Nonparametic Ranking Test

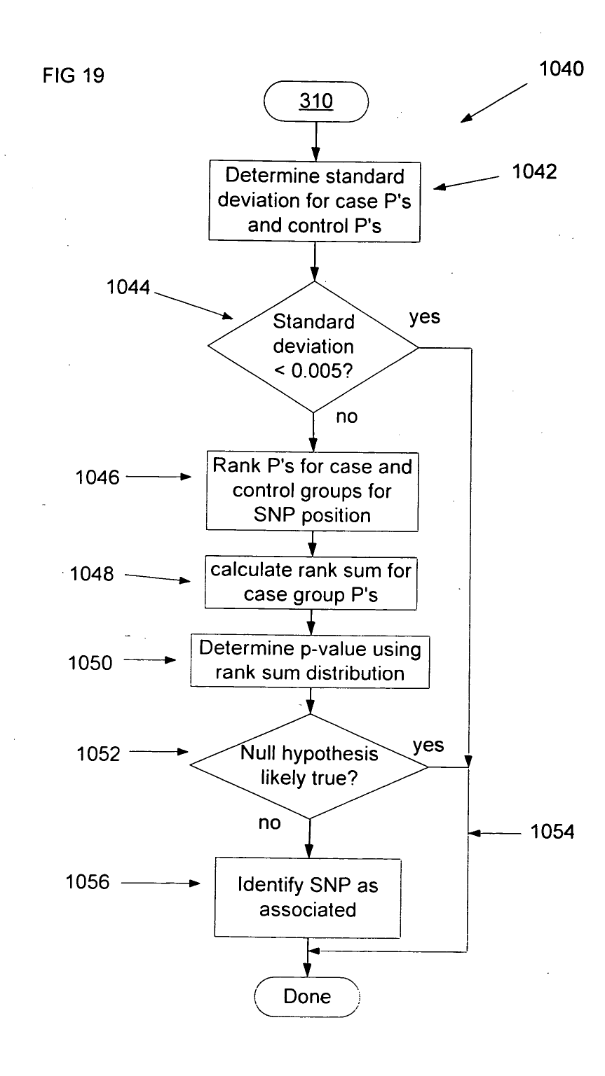

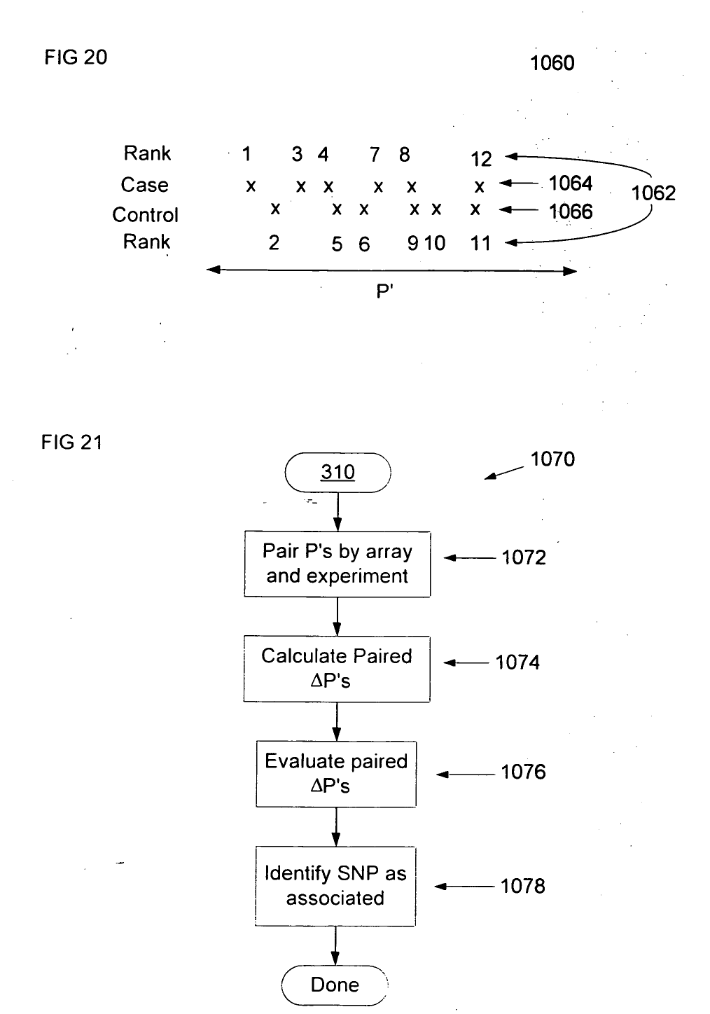

A further process 1040 for analyzing P′ data to identify associated SNPs, corresponding to method step 310, will now be described with reference to FIGS. 19 and 20. This process 1040 uses a nonparametric ranking test to identify SNPs which can be characterized as likely being associated SNPs. In one embodiment, the process uses a Wilcoxson rank-sum test (also referred to as a Mann-Whitney test) to obtain a p-value for the null hypothesis that the case and control group P′ values for a SNP from repeated experiments come from same distribution. A two-sided rank test is used as P′ case≠P′ control is indicative of an associated SNP. An advantage of a nonparametric test is that it is not predicated on the nature of the distribution of the P′ values from the repeated experiments.

In certain embodiments, the P′ analysis process 1040 includes some optional steps for determining whether the P′ data is suitable for analysis by the rank-sum test. The process calculates the standard deviation for the set of P′ values from repeated experiments for a SNP position for the case group and the control group. For a small set of P′ values resulting from a small number of repeated experiments, as there is a small sample size, there is a relatively large chance that the P′ values for the case group, or control group, would all be very similar, even though in reality the underlying distribution of P′ values is much greater. In order to reduce false positive identifications of associated SNPs from small P′ data sets, the standard deviation of the set of repeated experiment P′ case values and of the set of repeated experiment P′ control values are thresholded at step 1044 to identify clusters of P′ values having a very narrow distribution, indicative of a chance or fluke result. In one embodiment a standard deviation of 0.005 is used as the threshold standard deviation value. If either the case or control group P′ standard deviation is less than approximately 0.005, then the corresponding SNP position is rejected from further consideration. Sets of P′ data that pass this clustering step are then analyzed to identify associated SNPs by the remainder of the process. This checking of the P′ data is optional and is particularly useful when small groups of data are used. Steps 1042 and 1044 can be omitted if the sample sizes are sufficiently large.

The P′ values for the case and control group are then ranked 1046 by their absolute values, as illustrated by FIG. 20. FIG. 20 shows a graphical depiction 1060 illustrating the ranks 1062 of a set of six P′ values for the case group 1064 and six P′ values for the control group 1066 from six repeated experiments for a SNP position. The twelve P′ values are ordered by absolute magnitude and assigned a rank from one to twelve. Then the process calculates 1048 the rank sum for a one of the case group or the control group. In the example embodiment, the rank-sum for the case group is calculated. The rank sum distribution for twelve data items is determined from a look up table and a p-value is determined using the rank sum distribution and the case group rank sum.

The p-value is then thresholded against a confidence level to determine 1052 whether the null hypothesis that the case and control P′ data items come from the same distribution is sufficiently likely to be true. If the null hypothesis is sufficiently likely, then the SNP is not characterized as an associated SNP, as indicated by process flow 1054. If the null hypothesis is determined at step 1052 to be sufficiently unlikely to be true, then the SNP can be identified at step 1056 as an associated SNP. In an embodiment a 95% confidence level in accepting the alternative hypothesis is used for the p-value threshold. A 99% confidence level can be used if greater stringency is required in the identification of associated SNPs, e.g., in analyzing the results of a second tier experiment. The process 1040 can then be repeated for all SNPs that require evaluation.

vii) Analysis Based on Pairing Individual P′ Case and Control Values

A further process 1070 for analyzing P′ data to identify associated SNPs, corresponding to method step 310, will now be described with reference to FIG. 21. The process 1070 involves pairing individual P′ case and control values based on details of the experiment by which the individual P′ values were obtained. P′ case and P′ control values for a SNP are experimentally paired, by pairing P′ values that are obtained from experiments having at least one experimental condition or property the same. For example, the case and control P′ values for a SNP can be paired based on: both P′ values being obtained from the same lot or batch of array wafers or chips; both P′ values being obtained from an experiment on samples from the PCR reactions run in parallel; and both P′ values being obtained from an experiment carried out by the same person.

Pairing P′ values by common experimental conditions helps to prevent variations in experimental techniques and procedures from causing variations in the P′ values. A variety of different experimental conditions which can affect the outcome of an experiment can be used as the criterion by which to pair P′ case and control values and the following is by way of example only.

In a pairing step 1072, the process 1070 identifies P′ case and control values for a SNP position which were measured using array wafers or chips from the same production batch or lot and for which the experiment was carried out by the same person. In this way the effect of variations in array properties (e.g. probe properties) between lots and variations in the individual experimental technique of different people can be reduced. The process the calculates at step 1074 the difference, ΔP′, for the experimentally paired P′ case and control values for the SNPs positions and for each of the repeated experiments. The paired ΔP′s are then evaluated at step 1076 by any of a number of methods as will be described in greater detail below. Following evaluation of the paired ΔP′s, associated SNP positions are identified at step 1078.

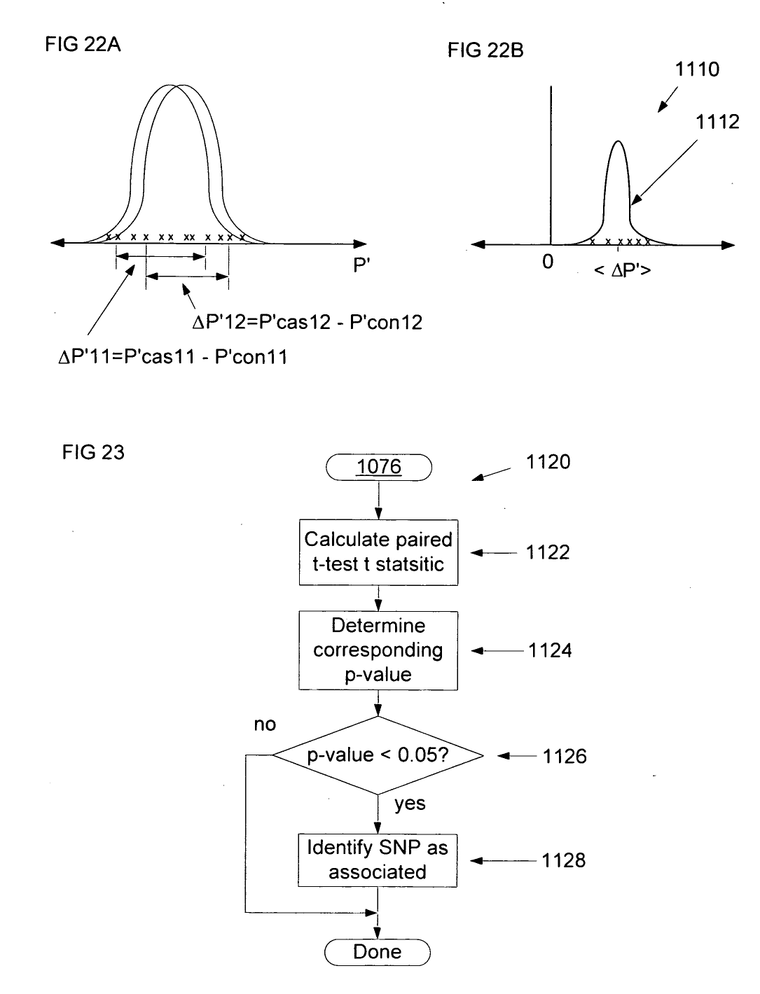

One process for evaluating the paired ΔP′s is to carry out a paired t-test using the paired P′ values. FIG. 22A shows a graphical representation 1100 of the distribution 1102 of a set of six P′ case values and a distribution 1104 of a set of six P′ control values for a SNP position. The paired t-test is similar to the t-test described previously but is carried out using pairs of data items from the two case and control group samples. The distributions of the case and control group P′ values may be sufficiently similar that a normal t-test may be unable to determine with sufficient confidence whether a single distribution or two different distributions underlies the measured P′ values. In this embodiment, the case and control groups samples were amplified in the PCR reactions run in parallel which were repeated to provide two samples distinguishable by the PCR reaction. The case and control sample were also labeled using different markers and hybridized together to a single chip or wafer. Three experiments were carried for each of the commonly amplified samples resulting in six P′ case and six P′ control values, as illustrated by x's 1106 in FIG. 22A.

The P′ case and P′ control values can then be paired by PCR reaction and experiment number to provide six experimentally paired P′s and corresponding ΔP′s. Two pairs are illustrated in FIG. 22A. ΔP′11 is the difference between the case P′ value for the first experiment using the sample from the first PCR reaction, P′cas11, and the control P′ value for the first experiment using the sample from the first PCR reaction, P′con11. ΔP′12 is the corresponding difference but for the second experiment using the first PCR reaction, and ΔP′13 for the third experiment using the first PCR reaction. Similarly ΔP′21 is the corresponding difference for the first experiment but using the second PCR reaction, ΔP′22 for the second experiment and ΔP′23 for the third experiment. FIG. 22B shows a graphical representation 1110 of the distribution 1112 of the set of six ΔP′s. A Paired t-test is used to determine the likelihood that the ΔP′s for the experimentally paired P′s have a non-zero value. Put another way, the paired t-test is used to determine the likelihood that the set of ΔP′s are not centered on zero.

FIG. 23 shows a flow chart illustrating a process 1120 for executing a paired t-test. The paired t-test is a test that the differences between pairs of observations are zero. The null hypothesis is therefore that ΔP′=0. The paired t-test t statistic is calculated at step 1122 using the following expression

$$t = \frac{〈 Δ {P}^{′} 〉}{\sqrt{\frac{{σ}_{Δ {P}^{′}}}{{N}_{Δ {P}^{′}}}}}$$

in which <ΔP′> is the arithmetic mean of the individual ΔP′s, σ_{ΔP′} is the standard deviation of the ΔP′s and N_{ΔP′} is the number of ΔP′s, which in this example is six. The p-value for the calculated t statistic is then determined at step 1124 from a look up table using the number of degrees of freedom, which is N_{ΔP′}−1 and being five in this example. The p-value is then used to determine at step 1126 whether the null hypothesis is sufficiently unlikely so that the alternate hypothesis ΔP′≠0 can be accepted. In one embodiment, a p-value of less than 0.05 is considered sufficient to reject the null hypothesis. Therefore, the p-value is thresholded at step 1126 and if the threshold is passed, the SNP is identified as an associated SNP 1128 (corresponding to step 1078 in FIG. 21). If the threshold is not met, then the SNP is not identified as an associated SNP.

The paired t-test is a parametric test and therefore is predicated on the data having a particular distribution. In instances in which a parametric test is not appropriate, a non-parametric counterpart to the paired t-test can be used, such as Friedman's test.

A number of other process can be used to implement step 1076 of evaluating the set of experimentally paired ΔP′s. In one process, the median value of the set of ΔP′s for a SNP position is selected and thresholded. If the median ΔP′ is equal to or greater than approximately 0.05 then the SNP is considered to be an associated SNP and is identified as such. In another process, an Olympic average is taken of the set of ΔP′s for a SNP. The largest and smallest ΔP′s are rejected and an average is taken of the remaining ΔP′s. The Olympic average ΔP′ is then thresholded, and the SNP is identified as an associated SNP if the Olympic average is equal to or greater than approximately 0.05. A further process involves determining whether the set of ΔP′s all have the same sign. If the set of ΔP′s (where all the ΔP′s have been calculated consistently) all have the same sign (i.e. all positive or all negative) then the consistent determination of ΔP′ across repeated experiments is an indication that the ΔP′ is ≠0 and so the SNP is identified as likely being an associated SNP. In a further process, a ranking test, similar to that described with reference to FIG. 19, can be used to evaluate the set of experimentally paired ΔP′s.

The above described processes provide example embodiments of methods for analyzing P′ values to determine whether a SNP position should be characterized as likely being associated or not. The processes use at least two measures of P′ or ΔP′ for a SNP position or multiple SNP positions, in order to characterize the SNP position. Various statistical tests have been described above and other parametric and nonparametric tests can be used depending on the P′ values available for analysis. Many of the processes use properties of the distribution of P′ or ΔP′ values, such as an arithmetic mean, standard deviation, median, etc, and other properties of distributions can be used. Various cut off values or thresholds are used in the processes which can be empirically determined and can vary depending on the experiments being carried out and/or on the required stringency. The use of genetic data to identify or validate SNP associations can also be used. A number of the steps used in the different example processes may be used in, or adapted or modified for use in, other of the example processes as will be apparent from the teachings herein.

Although the foregoing invention has been described in some detail for purposes of clarity of understanding, it will be apparent that certain changes and modifications may be practiced within the scope of the appended claims. Therefore, the described embodiments should be taken as illustrative and not restrictive, and the invention should not be limited to the details given herein but should be defined by the following claims and their full scope of equivalents.

1. A computer-implemented method for characterizing an interrogation position in a nucleic acid segment, comprising:
inputting into a computer system a first measure of relative allele frequency at the interrogation position in the nucleic acid segment derived from a first sample collected from a first group of n individuals, wherein n is an integer equal to or larger than 2;
inputting into the computer system a second measure of relative allele frequency at the interrogation position in the nucleic acid segment derived from a second sample collected from a second group of m individuals, wherein m is an integer equal to or larger than 2; and
analyzing in the computer system the first measure and the second measure to characterize the interrogation position.

2. The method of claim 1, wherein the first group is a case group and the second group is a control group.

3. The method of claim 2, wherein the individuals in the first and second groups are animals.

4. The method of claim 2, wherein the individuals in the first and second groups are mammals.

5. The method of claim 2, wherein the individuals in the first and second groups are humans.

6. The method of claim 2, wherein the individuals in the case group are humans who are selected based on a phenotypic characteristic of interest; the individuals in the control group are humans who are selected based on lack of the phenotypic characteristic of interest; and the step of analyzing includes analyzing in the computer system the first measure and the second measure to characterize the interrogation position as being associated with the phenotypic characteristic of interest.

7. The method of claim 6, wherein the phenotypic characteristic of interest is susceptibility or resistance to a disease, a disorder, or infection of a pathogen.

8. The method of claim 6, wherein the phenotypic characteristic of interest is susceptibility or resistance to effects of uptake of food or drink.

9. The method of claim 8, wherein the drink is an alcoholic drink.

10. The method of claim 6, wherein the phenotypic characteristic of interest is susceptibility to, resistance to, or adverse effects of a therapy using a therapeutic agent or a medical device.

11. The method of claim 6, wherein phenotypic characteristic of interest is selected from the group consisting of cancer, hematological disorders, autoimmune diseases, inflammatory diseases, cardiovascular diseases, liver diseases, neurodegenerative diseases, diabetes, kidney disorder, gastrointestinal disorders, pain, bacterial infection, parasitic infection, viral infection, and a specific stage of development thereof.

12. The method of claim 6, wherein the number of individuals in the case group, n, and the number of the individuals in the control group, m, are each larger than 5.

13. The method of claim 6, wherein the number of individuals in the case group, n, and the number of the individuals in the control group, m, are each larger than 100.

14. The method of claim 6, wherein the number of individuals in the case group, n, and the number of the individuals in the control group, m, each independently varies between 10-100,000.

15. The method of claim 6, wherein the interrogation position is a SNP position.

16. The method of claim 15, wherein the genetic region containing the SNP position was not previously known to be associated with the phenotypic characteristic of interest.

17. The method of claim 1, wherein the first and second samples are each collected by pooling biological samples from the first and second group of individuals, respectively.

18. The method of claim 17, wherein the biological samples are pooled by combining a substantially equal amount of biological sample from each individual in each group.

19. The method of claim 17, wherein the biological samples are selected from the group consisting of genomic DNA, mitochondrial DNA, extragenomic DNA, cDNA, and RNA.

20. The method of claim 17, wherein the biological samples from the first and second groups are labeled with a detectable marker.

21. The method of claim 20, wherein the detectable marker is selected from the group consisting of cychrome, fluorescein, Alexa-488, radioisotopes, and biotin.

22. The method of claim 1, wherein the first sample is collected by pooling genetic material from the first group of individuals and amplifying the pooled genetic materials in the first group and the second sample is collected by pooling genetic material from the second group of individuals and amplifying the pooled genetic materials in the second group.

23. The method of claim 22, wherein amplicons of genetic material from the first group is labeled with a different detectable marker than are amplicons of genetic material from the second group.

24. The method of claim 23, wherein amplicons of genetic materials from the first group are each labeled with biotin; and amplicons of genetic materials from the second group are each labeled with fluorescein.

25. The method of claim 23, wherein the first sample and second sample are mixed after being labeled, followed by hybridization to an array and subsequent staining, wherein said first sample is stained with different stain than said second sample.

26. The method of claim 1, wherein the first sample is collected by amplifying genetic material from each individual in the first group of individuals and then pooling the amplified genetic materials from all of the individuals in the first group, and the second sample is collected by amplifying genetic material from each individual in the second group of individuals and then pooling the amplified genetic materials from all of the individuals in the second group.

27. The method of claim 1, wherein the interrogation position contains a biallelic polymorphism.

28. The method of claim 1, wherein the first measure of relative allele frequency is derived from a first measure of intensity of signals from a first probe set on a first oligonucleotide array; and the second measure of relative allele frequency is derived from a second measure of intensity of signals from a second probe set on the first or a second oligonucleotide array.

29. The method of claim 28, wherein the density of the oligonucleotide array is at least 100 probes per square centimeters.

30. The method of claim 28, wherein the density of the oligonucleotide array is at least 1000 probes per square centimeters.

31. The method of claim 28, wherein the density of the oligonucleotide array is between 100-100,000,000 probes per square centimeters.

32. The method of claim 28, wherein the density of the oligonucleotide array is between 1,000,000-80,000,000 probes per square centimeters.

33. The method of claim 28, wherein each of the oligonucleotides on the array is 10-100 nucleotides in length.

34. The method of claim 28, wherein each of the oligonucleotides on the array is 20-50 nucleotides in length.

35. The method of claim 28, further comprising:
correcting the first and second measures of intensity.

36. The method of claim 35, wherein the step of correcting includes subtracting a background intensity.

37. The method of claim 36, wherein the background intensity is the intensity of a probe cell having the 1000^{th} lowest intensity on the oligonucleotide array.

38. The method of claim 36, wherein the background intensity is determined by calculating an equation of the form:
$$\frac{〈 {I}_{R}^{m m} 〉 + 〈 {I}_{A}^{m m} 〉}{2} .$$

39. The method of claim 28, further comprising:
evaluating the first and second measures of intensity to determine whether the first probe set and the second probe set have each detected the nucleotide sequences that they were designed to detect.

40. The method of claim 39, wherein the step of evaluating includes determining if the measure of intensity of a perfectly complementary probe in the first probe set is greater than any of the measures of intensity of mismatch probes in the first probe set, in which case the first probe set is a conforming probe set; and determining if the measure of intensity of a perfectly complementary probe in the second probe set is greater than any of the measures of intensity of mismatch probes in the second probe set, in which case the second probe set is a conforming probe set

41. The method of claim 28, wherein the first or second probe set is included in a probe tiling that comprises
a reference tiling comprising a set of reference oligonucleotide probes with a varying nucleotide at the interrogation position and a set of complementary reference oligonucleotide probes that are each complementary to the corresponding reference oligonucleotide probes; and
an alternate tiling comprising a set of alternate oligonucleotide probes with a varying nucleotide at the interrogation position and a set of complementary alternate oligonucleotide probes that are each complementary to the corresponding alternate oligonucleotide probes.

42. The method of claim 41, wherein the reference and alternate oligonucleotide probes differ from each other by a nucleotide at the interrogation position.

43. The method of claim 41, wherein the varying nucleotide at the interrogation position is A, T, G, U or C.

44. The method of claim 43, further comprising:
calculating a number of conforming probe sets for the reference tiling;
calculating a total number of probe sets in the reference tiling;
calculating a conformance value for the reference tiling by dividing the number of conforming probe sets with the total number of probe sets in the reference tiling;
calculating a number of conforming probe sets for the alternate tiling;
calculating a total number of probe sets in the alternate tiling; and
calculating a conformance value for the alternate tiling by dividing the number of conforming probe sets with the total number of probe sets in the alternate tiling.

45. The method of claim 44, further comprising:
discarding any measure of intensity obtained from the tiling that has the conformance value lower than 0.6.

46. The method of claim 44, further comprising:
discarding any measure of intensity obtained from the tiling that has the conformance value lower than 0.9.

47. The method of claim 28, wherein said first probe set and said second probe set are the same probe set.

48. The method of claim 1, wherein the relative allele frequency is based on variation of a nucleotide at the interrogation position of the nucleic acid segment relative to a reference nucleic acid segment, the nucleotide at said interrogation position in said reference nucleic acid segment being designated a reference allele and a nucleotide at said interrogation position that differs from said reference allele being designated an alternate allele; and the measure of relative allele frequency is the proportion of either said reference allele or said alternate allele at the interrogation position.

49. The method of claim 48, wherein the relative allele frequency is determined by calculating an equation of the form:
cᵣ/(cₐ+cᵣ) or
cₐ/(cₐ+cᵣ),
where cᵣ is the concentration of the reference allele and cₐ is the concentration of the alternate allele.

50. The method of claim 48, wherein the measure of relative allele frequency is determined by calculating an equation of the form:
I_{R}/(I_{A}+I_{R}) or
I_{A}/(I_{A}+I_{R}),
where I_{R} is an intensity of signal from the reference allele, and I_{A} is an intensity of signal from the alternate allele.

51. The method of claim 48, wherein the measure of relative allele frequency is determined using measurements of hybridization to perfect match probes.

52. The method of claim 48, wherein each measure of relative allele frequency is determined using at least two intensity of signal measurements by calculating at least one of the equations of the form:
<I_{R}>/(<I_{A}>+<I_{R}>) and
<I_{A}>/(<I_{A}>+<I_{R}>),
where <I_{R}> is an average of intensities of signal measurements from the reference allele, and <I_{A}> is an average of intensities of signal measurements from the alternate allele.

53. The method of claim 52, wherein the averages of intensities of signal measurements are arithmetic means of intensities of signal measurements.

54. The method of claim 52, wherein the averages of intensities of signal measurements are trimmed means of intensities of signal measurements.

55. The method of claim 48, wherein each measure of relative allele frequency is determined using at least two intensity of signal measurements by calculating at least one of the equations of the form:
$$\begin{matrix}< {I}_{R} / ( {I}_{A} + {I}_{R} ) >= \frac{{\sum }_{i = 1}^{n} ( \frac{{I}_{R}}{{I}_{A} + {I}_{{R}_{i}}} )}{n} and \\ < {I}_{A} / ( {I}_{A} + {I}_{R} ) >= \frac{{\sum }_{i = 1}^{n} ( \frac{{I}_{A}}{{I}_{A} + {I}_{{R}_{i}}} )}{n} ,\end{matrix}$$
where I_{R} is an intensity of signal measurement from the reference allele, I_{A} is an intensity of signal measurement from the alternate allele, <I_{R}/(I_{A}+I_{R})> and <I_{A}/(I_{A}+I_{R})> are averages of the ratios of intensities of signal measurements, and n is a number of offsets at which the intensity of signal measurements were measured.

56. The method of claim 55, wherein the averages of the ratios of intensities of signal measurements are arithmetic means of the ratios of intensities of signal measurements.

57. The method of claim 55, averages of the ratios of intensities of signal measurements are trimmed means of the ratios of intensities of signal measurements.

58. The method of claim 48, wherein wherein each measure of relative allele frequency is determined using at least one intensity of signal measurement that has been corrected.

59. The method of claim 58, wherein the at least one intensity of signal measurement is corrected by subtracting background.

60. The method of claim 59, wherein the background is calculated at least from intensity of signal measurements from mismatch probes.

61. The method of claim 60, wherein the background is calculated from a mean of the intensity of signal measurements from mismatch probes.

62. The method of claim 61, wherein the mean of intensities of signal measurements from mismatch probes is an arithmetic mean.

63. The method of claim 61, wherein the mean of intensities of signal measurements from mismatch probes is a trimmed mean.

64. The method of claim 1, wherein the step of analyzing the first measure and the second measure includes determining a difference between the first measure and the second measure.

65. The method of claim 64, further comprising:
determining if the difference falls within a predetermined percentile of a distribution of differences between first measures and second measures.

66. The method of claim 65, wherein the percentile of distribution is top 20%.

67. The method of claim 65, wherein the percentile of distribution is top 5%.

68. The method of claim 1, further comprising:
inputting into the computer system a further first measure of relative allele frequency that is obtained by repeating the measurement of the first measure of relative allele frequency; and
inputting into the computer system a further second measure of relative allele frequency that is obtained by repeating the measurement of the second measure of relative allele frequency, wherein the step of analyzing includes determining, based on the variation in the first measure and further first measure and the variation in the second measure and further second measure, whether the first, further first, second and further second measures of relative allele frequency are suitable for use in characterizing the interrogation position.

69. The method of claim 6, wherein the interrogation position is a SNP position at position i within a haplotype block having N different haplotype patterns, and each of the first and second measures of relative allele frequency is corrected by applying a first equation:
$${P}_{i} \approx {\sum }_{j = 1}^{N} {M}_{ij} {f}_{j}$$
where Pᵢ is a corrected relative allele frequency of the interrogation position; N is the total number of different haplotype patterns within the haplotype block; mᵢⱼ is a coefficient having a value of +1 if the allele at position i matches a reference allele of the SNP and having a value of 0 if the allele at position i matches an alternate allele of the SNP; and fⱼ is a haplotype pattern frequency.

70. The method of claim 69, wherein the first equation is constrained by a second equation:
$${\sum }_{j = 1}^{N} {f}_{j} = 1.$$

71. The method of claim 70, wherein the fⱼ has a value ranging from 0 to 1.

72. The method of claim 1, further comprising:
calculating a difference between the first measure of relative allele frequency and the second measure of relative allele frequency.

73. The method of claim 72, wherein the interrogation position is a SNP position at position i within a haplotype block having N different haplotype patterns, and the difference between the first measure and second measure of relative allele frequency is corrected by applying a third equation:
$$Δ {P}_{i} \approx {\sum }_{j = 1}^{N} {m}_{ij} Δ {f}_{j}$$
where ΔPᵢ is a corrected relative allele frequency difference of the interrogation position; N is the total number of different haplotype patterns within the haplotype block; mᵢⱼ is a coefficient having a value of +0.5 if the allele at position i matches a reference allele of the SNP and having a value of −0.5 if the allele at position i matches an alternate allele of the SNP; and Δfⱼ is a haplotype pattern frequency difference.

74. The method of claim 73, wherein the third equation is constrained by a fourth equation:
$${\sum }_{j = 1}^{N} {f}_{j} = 0.$$

75. The method of claim 6, wherein the step of inputting the first measure includes inputting a plurality of first measures, and the step of inputting the second measure includes inputting a plurality of second measures.

76. The method of claim 75, wherein the plurality of first measures and the plurality of second measures correspond to duplicated or replicated measurements on the same interrogation position in the nucleic acid segment.

77. The method of claim 1, wherein said interrogation position is a plurality of interrogation positions in the same or different nucleic acid segments, and for each interrogation position a said first measure of allele frequency and a said second measure of allele frequency are inputted into said computer system and analyzed to characterize said each interrogation position.

78. The method of claim 75, wherein the step of analyzing includes pairing a measure from the plurality of first measures and a measure from the plurality of the second measures based on common experimental conditions.

79. The method of claim 75, wherein the step of analyzing includes analyzing using a set of differences of paired measures using a method selected from the group comprising: a paired t-test; calculating an Olympic average; determining the median value; and all members of the set having the same sign.

80. The method of claim 75, where the step of analyzing includes calculating the mean of the plurality of first measures and the mean of the plurality of second measures.

81. The method of claim 80, further comprising:
analyzing the absolute difference between the mean of the plurality of first measures and the mean of the plurality of second measures by thresholding the difference between the means, wherein if the absolute difference between the mean of the plurality of first measures and the mean of the plurality of second measures is equal to or above the threshold value, the interrogation position is characterized as being associated with the phenotypic characteristic of interest.

82. The method of claim 81, wherein the threshold value is 0.04.

83. The method of claim 81, wherein the threshold value is 0.1.

84. The method of claim 81, further comprising:
inputting a further plurality of first measures and a further plurality of second measures;
calculating a mean of the further plurality of first measures and a mean of the further plurality of second measures;
analyzing the difference between the mean of the further plurality of first measures and the mean of the further plurality of second measures by using a further threshold value.

85. The method of claim 84, wherein the threshold value is 0.05 and the further threshold value is 0.10, wherein if the absolute difference between the mean of the further plurality of first measures and the mean of the further plurality of second measures is equal to or above the further threshold value, the interrogation position is characterized as being associated with the phenotypic characteristic of interest.

86. The method of claim 75, wherein the step of analyzing includes:
calculating the standard deviation of the plurality of first measures and the standard deviation of the plurality of second measures; and
analyzing each of the standard deviations of the first and second pluralities of measures.

87. The method of claim 86, wherein each of the standard deviations of the first and second pluralities of measures is analyzed by using a chi-squared distribution to determine at least one cutoff value for the standard deviations.

88. The method of claim 87, further comprising:
discarding the plurality of the first or second measures if the calculated standard deviation of the plurality of the first or second measures is greater than the cutoff value.

89. The method of claim 75, further comprising:
calculating an arithmetic mean and standard deviation of the plurality of the first measures, and an arithmetic mean and standard deviation of the plurality of second measures, and
applying a t-test using said arithmetic means and standard deviations to determine whether it is likely that the plurality of first measures and the plurality of second measures are from the same or different distributions, where if the plurality of first measures and the plurality of second measures are found likely to be from different distributions, the interrogation position in the nucleic acid segment is characterized as being associated with the phenotypic characteristic of interest.

90. The method of claim 75, further comprising:
calculating an arithmetic mean and standard deviation for the plurality of the first measures, and an arithmetic mean and standard deviation for the plurality of second measures,
determining a distribution for the plurality of first measures based on said arithmetic mean and standard deviation of the plurality of first measures,
determining a distribution for the plurality of second measures based on said arithmetic mean and standard deviation of the plurality of second measures, and
determining a difference between said distribution for the plurality of first measures and said distribution for the plurality of second measures, wherein if the difference between said distribution of the plurality of first measures and said distribution of the plurality of second measures is significant, either positive or negative, the interrogation position in the nucleic acid segment is characterized as being associated with the phenotypic characteristic of interest.

91. The method of claim 89, wherein the likelihood that the plurality of first measures and the plurality of second measures are from different distributions is assessed by using a formula of the form:
$$t = \frac{< {P}_{1}^{′} > - < {P}_{2}^{′} >}{\sqrt{\frac{{σ}_{1}^{2}}{{N}_{1}} + \frac{{σ}_{2}^{2}}{{N}_{2}}}}$$
where t is the t-statistic in the t-test analysis; <P′₁> and <P′₂> are arithmetic means of the pluralities of first and second measures, respectively; σ₁ and σ₂ are standard deviations of the pluralities of first and second measures, respectively; and N₁ and N₂ are numbers of members of the pluralities of first and second measures, respectively.

92. The method of claim 89, further comprising:
comparing the t-statistic with a Student t-distribution using a number of degrees of freedom, which is calculated by using an equation of the form (N₁+N₂−2), to obtain a p-value, where if the p-value is lower than 0.05, the interrogation position in the nucleic acid segment is characterized as being associated with the phenotypic characteristic of interest.

93. The method of claim 92, further comprising:
repeating the same steps for characterization of the interrogation position in the nucleic acid segment on one or more further interrogation positions in the same or different nucleic acid segment;
discarding the interrogation positions having p-values higher than a cutoff value, and characterizing the remaining interrogation position(s) as being associated with the phenotypic characteristic of interest.

94. The method of claim 75, further comprising:
calculating absolute values of the plurality of the first measures and calculating absolute values of the plurality of the second measures;
ranking the absolute values of the plurality of the first measures; ranking the absolute values of the plurality of the second measures; and
determining the rank-sum distribution for both the plurality of the first measures and the plurality of the second measures.

95. The method of claim 94, wherein the rank-sum distribution is determined by using a Wilcoxson rank-sum test.

96. The method of claim 95, further comprising:
determining the p-value for the interrogation position, wherein a confidence level higher than a selected value indicates that the interrogation position is a SNP position associated with the phenotypic characteristic of interest.

97. The method of claim 95, further comprising:
determining the p-value for the interrogation position, wherein a confidence level higher than 99% indicates that the interrogation position is a SNP position associated with the phenotypic characteristic of interest.

98. The method of claim 6, wherein said an interrogation position is at least two different interrogation positions and the step of inputting the first measure includes i) inputting a plurality of first measures corresponding to the different interrogation positions, and the step of inputting the second measure includes ii) inputting a plurality of second measures corresponding to the same interrogation positions as in step i).

99. The method of claim 98, further comprising:
iii) repeating step i) to obtain a further plurality of first measures; and
iv) repeating step ii) to obtain a further plurality of second measures.

100. The method of claim 99, further comprising:
calculating the difference between the plurality of the first measures and the plurality of the second measures for each of the different interrogation positions;
identifying the interrogation positions having a difference above a first threshold value, and the interrogation positions having a difference below a second threshold value between the plurality of the first measures and the plurality of the second measures;
calculating the difference between the further plurality of the first measures and the further plurality of the second measures for each of the different interrogation positions;
identifying the interrogation positions having a difference above the first threshold value, and the interrogation positions having a difference below the second threshold value between the further plurality of the first measures and the further plurality of the second measures,
characterizing those interrogation positions that are identified as having a difference above the first threshold value in both the plurality and further plurality of measures as being associated with the phenotypic characteristic of interest, and
characterizing those interrogation positions that are identified as having a difference below the second threshold value in both the plurality and further plurality of measures as being associated with the phenotypic characteristic of interest.

101. The method of claim 100, wherein the first threshold value separates the top 20% in the distribution of the calculated differences from the bottom 95%, and the second threshold value separates the bottom 20% in the distribution of the calculated differences from the top 95%, wherein those interrogation positions that have a difference in the top 20% or the bottom 20% of the distribution of calculated differences are characterized as associated.

102. The method of claim 100, wherein the first threshold value separates the top 5% in the distribution of the calculated differences from the bottom 95%, and the second threshold value separates the bottom 5% in the distribution of the calculated differences from the top 95%, wherein those interrogation positions that have a difference in the top 5% or the bottom 5% of the distribution of calculated differences are characterized as associated.

103. The method of claim 6, further comprising:
validating the characterization of the interrogation position.

104. The method of claim 103, wherein the step of validating includes identifying a location of the nucleic acid segment in the human genome.

105. The method of claim 104, wherein if the nucleic acid segment is located in a coding or regulatory region of a gene and the interrogation position was designated as associated with the phenotypic characteristic of interest, then the gene is deemed to be associated with the phenotypic characteristic of interest and the step of validating further includes cloning and expressing the associated gene to produce a protein product and characterizing the protein product.

106. The method of claim 104, wherein if the nucleic acid segment is located in a coding or regulatory region of a gene, and the interrogation position was designated as associated with the phenotypic characteristic of interest, then the gene is deemed to be associated with the phenotypic characteristic of interest and the step of validating further includes regulating expression of the associated gene in cells in vitro or in vivo and detecting changes of cells in response to the regulation.

107. The method of claim 104, wherein if the nucleic acid segment is located in a coding or regulatory region of a gene and the interrogation position was designated as associated with the phenotypic characteristic of interest, then the gene is deemed to be associated with the phenotypic characteristic of interest and the step of validating further includes:
screening a library of pharmaceutical candidates against the associated gene or gene product; and
selecting the pharmaceutical candidates that modulate expression of the associated gene or activity of the associated gene product.

108. A method for determining a relative allele frequency for an interrogation position in a nucleic acid segment based on nucleotide variation at the interrogation position of the nucleic acid segment relative to a reference nucleic acid segment and the nucleic acid segment with nucleotide variation being designated as an alternate nucleic acid segment, comprising:
determining a plurality of intensities of signals from the reference nucleic acid segment, which are designated as I_{R,1-i} wherein i is an integer equal to or larger than 2;
determining a plurality of intensities of signals from the alternate nucleic acid segment, which are designated as I_{A,1-j} wherein j is an integer equal to or larger than 2;
determining the relative allele frequency of an interrogation position by calculating an equation of the form:
<I_{R,1-j}>/(<I_{A,1-j}>+<I_{R,1-i}>) or
<I_{A,1-j}>/(<I_{A,1-j}>+<I_{R,1-i}>),
where <I_{R,1-i}> is the average of the plurality of intensities of signals from the reference nucleic acid segment, and <I_{A,1-j}> is the average of intensities of signals from the alternate nucleic acid segment.

109. The method of claim 108, wherein the signals result at least from perfect match probes on an oligonucleotide array.

110. The method of claim 108, wherein the average of intensities of signals is an arithmetic mean of intensities of signals.

111. The method of claim 108, wherein the average of intensities of signals is a trimmed mean of intensities of signals.

112. The method of claim 108, wherein the average of the plurality of intensities of signals from the reference nucleic acid segment, <I_{R,1-i}>, excludes outlier signals; and the average of the plurality of intensities of signals from the alternate nucleic acid segment, <I_{A,1-j}>, excludes outlier signals.

113. The method of claim 108, wherein at least one of <I_{R,1-i}> or <I_{A,1-j}> is corrected.

114. The method of claim 113, wherein at least one of <I_{R,1-i}> or <I_{A,1-j}> is corrected for background.

115. The method of claim 114, wherein at least one of <I_{R,1-i}> or <I_{A,1-j}> are corrected for background and the relative allele frequency of an interrogation position is determined by calculating an equation of the form:
$$\frac{( < {I}_{R}^{pm} > - \frac{< {I}_{R}^{mm} > + < {I}_{A}^{mm} >}{2} )}{\begin{matrix}( < {I}_{A}^{pm} > - \frac{< {I}_{R}^{mm} > + < {I}_{A}^{mm} >}{2} ) + \\ ( < {I}_{R}^{pm} > - \frac{< {I}_{R}^{mm} > + < {I}_{A}^{mm} >}{2} )\end{matrix}}$$
wherein <I_{R}^{pm}> is the average of the plurality of intensities of signals from the reference nucleic acid segment generated by perfect match probes on an oligonucleotide array; <I_{A}^{pm}> is the average of the plurality of intensities of signals from the alternate nucleic acid segment generated by perfect match probes on the oligonucleotide array; <I_{R}^{mm}> is the average of the plurality of intensities of signals from the reference nucleic acid segment generated by mismatch probes on the oligonucleotide array; and <I_{A}^{mm}> is the average of the plurality of intensities of signals from the alternate nucleic acid segment generated by mismatch probes on the oligonucleotide array.

116. The method of claim 115, wherein the average of intensities of signals is an arithmetic mean of intensities of signals.

117. The method of claim 115, wherein the average of intensities of signals is a trimmed mean of intensities of signals.

118. A computer-implemented method for characterizing a polymorphic marker in a nucleic acid, comprising:
inputting into a computer system a first measure of relative allele frequency for the polymorphic marker in a first sample, said first sample containing nucleic acids from a first group of n individuals, wherein n is an integer equal to or larger than 2;
inputting into a computer system a second measure of relative allele frequency for the polymorphic marker in a second sample, said second sample containing nucleic acids from a second group of m individuals, wherein m is an integer equal to or larger than 2; and
analyzing in the computer system the first measure and the second measure to characterize the polymorphic marker as being associated with a phenotypic trait of interest.

119. A computer-implemented method for characterizing an interrogation position in a nucleic acid segment, comprising:
inputting into a computer system a group of first measures of hybridization probe intensities corresponding to the interrogation position in the nucleic acid segment derived from a first sample collected from a case group of n individuals, wherein n is an integer equal to or larger than 2;
calculating in the computer system a group of first relative allele frequencies at the interrogation position for the case group based on the group of first measures of hybridization probe intensities;
inputting into the computer system a group of second measures of hybridization probe intensities corresponding to the interrogation position in the nucleic acid segment derived from a second sample collected from a control group of m individuals, wherein m is an integer equal to or larger than 2;
calculating in the computer system a group of second relative allele frequencies at the interrogation position for the control group based on the group of second measures of hybridization probe intensities;
analyzing in the computer system the group of the first relative allele frequencies and the group of second relative allele frequencies to characterize the interrogation position as being associated with a phenotypic characteristic of interest.

120. The method of claim 119, wherein the group of the first measures of hybridization probe intensities are obtained by at least 2 sets of repetitive experiments, and the group of the second measures of hybridization probe intensities are obtained by at least 2 sets of repetitive experiments.

121. The method of claim 119, wherein the step of analyzing includes:
calculating a mean of the group of the first relative allele frequencies;
calculating a mean of the group of the second relative allele frequencies; and
calculating the absolute difference between the mean of the group of the first relative allele frequencies and the mean of the group of the second relative allele frequencies, where if the absolute difference is equal to or above a predetermined threshold value, the interrogation position is characterized as being associated with the phenotypic characteristic of interest.

122. The method of claim 121, wherein the threshold value ranges from 0.02 to 0.2.

123. The method of claim 121, wherein the threshold value ranges from 0.04 to 0.1.

124. The method of claim 119, wherein the step of analyzing includes applying a statistical test to the group of the first relative allele frequencies and the group of second relative allele frequencies to characterize the interrogation position as being associated with a phenotypic characteristic of interest.

125. The method of claim 124, wherein the statistical test is a t-test or rank-sum test.

126. The method of claim 119 wherein said an interrogation position is at least two interrogation positions, and further comprising:
calculating the difference between the group of the first measures and the group of the second measures for each of the different interrogation positions;
characterizing the interrogation positions having a difference above a first threshold value, and interrogation positions having a difference below a second threshold value between the group of the first measures and the group of the second measures as associated with the phenotypic characteristic of interest.

127. A computer-implemented method for characterizing an interrogation position in a nucleic acid segment with a previously known location in the human genome, comprising:
inputting into a computer system a first measure of relative allele frequency at the interrogation position in the nucleic acid segment derived from a first sample collected from a first group of n individuals, wherein n is an integer equal to or larger than 2;
inputting into the computer system a second measure of relative allele frequency at the interrogation position in the nucleic acid segment derived from a second sample collected from a second group of m individuals, wherein m is an integer equal to or larger than 2; and
analyzing in the computer system the first measure and the second measure to characterize the interrogation position.

128. The method of claim 127, wherein analyzing includes analyzing in the computer system the first measure and the second measure to characterize the interrogation position as being associated with a phenotypic characteristic of interest.

129. The method of claim 128, wherein the nucleotide segment is proximal to or within a region of a candidate gene that is not previously known to be associated the phenotypic characteristic of interest.

130. The method of claim 128, wherein the nucleotide segment is proximal to or within a region of a candidate gene that is previously suspected of being associated the phenotypic characteristic of interest.

131. The method of claim 128, wherein the nucleotide segment is proximal to or within an untranslated region of a candidate gene that is not previously known to be associated the phenotypic characteristic of interest.

132. The method of claim 128, wherein the nucleotide segment is proximal to or within an untranslated region of a candidate gene that is previously suspected of being associated the phenotypic characteristic of interest.

133. Data processing apparatus for characterizing an interrogation position in a nucleic acid segment, comprising:
a data processor;
a storage device holding computer readable code in communication with the data processor, the computer readable code including:
computer code which determines a first measure of relative allele frequency at the interrogation position in the nucleic acid segment derived from a first sample collected from a first group of n individuals, wherein n is an integer equal to or larger than 2;
computer code which determines a second measure of relative allele frequency at the interrogation position in the nucleic acid segment derived from a second sample collected from a second group of m individuals, wherein m is an integer equal to or larger than 2; and
computer code which analyzes the first measure and the second measure to characterize the interrogation position.

134. The data processing apparatus of claim 133, wherein analyzing includes analyzing in the first measure and the second measure to characterize the interrogation position as being associated with a phenotypic characteristic of interest.

135. The data processing apparatus of claim 134, wherein the data processing apparatus is further in communication with another data storage device which stores a first measure of the intensity of signals from a first probe set on an oligonucleotide array and a second measure of the intensity of signals from a second probe set on the oligonucleotide array, wherein the first measure of relative allele frequency is determined based on the first measure of intensity and the second measure of relative allele frequency is determined based on the second measure of intensity.

136. The data processing apparatus of claim 135, wherein the data processing apparatus is further in communication with an imaging device.

137. The data processing apparatus of claim 136, wherein the imaging device is a scanner that determines the first measure of intensity and the second measure of intensity.

138. A computer readable medium holding computer readable code for characterizing a position in a nucleic acid segment and for carrying out the processes of:
determining a first measure of relative allele frequency at the interrogation position in the nucleic acid segment derived from a first sample collected from a first group of n individuals, wherein n is an integer equal to or larger than 2;
determining a second measure of relative allele frequency at the interrogation position in the nucleic acid segment derived from a second sample collected from a second group of m individuals, wherein m is an integer equal to or larger than 2; and
analyzing the first measure and the second measure to characterize the interrogation position.

139. The computer readable medium of claim 138, wherein analyzing includes analyzing the first measure and the second measure to characterize the interrogation position as being associated with a phenotypic characteristic of interest.


SEQUENCE LISTING (4 sequences)

SEQ ID NO: 1 - DNA, 10 bp, Artificial Sequence
  haplotype pattern from human genomic DNA
actactagat

SEQ ID NO: 2 - DNA, 10 bp, Artificial Sequence
  haplotype pattern from human genomic DNA
gtggacgact

SEQ ID NO: 3 - DNA, 10 bp, Artificial Sequence
  haplotype pattern from human genomic DNA
gcggacgact

SEQ ID NO: 4 - DNA, 10 bp, Artificial Sequence
  haplotype pattern from human genomic DNA
actactagcg